(19) United States
(12) Patent Application Publication
(10) Pub. No.: US 2012/0023616 A1
(43) Pub. Date: Jan. 26, 2012

(54) METHODS FOR PREVENTING OR INHIBITING MICROBIAL INFECTION OF PLANTS AND PLANT EXHIBITING RESISTANCE TO MICROBIAL INFECTION

(75) Inventors: Marie Nishimura, Ibaraki (JP); Yoko Nishizawa, Ibaraki (JP); Takashi Fujikawa, Ibaraki (JP); Ichiro Mitsuhara, Ibaraki (JP); Eiichi Minami, Ibaraki (JP); Keietsu Abe, Miyagi (JP); Takashi Tachiki, Shiga (JP); Shigekazu Yano, Shiga (JP)

(73) Assignee: NATIONAL INSTITUTE OF AGROBIOLOGICAL SCIENCES, Tsukuba-shi, Ibaraki (JP)

(21) Appl. No.: 13/256,904

(22) PCT Filed: Mar. 16, 2010
(86) PCT No.: PCT/JP2010/054906

§ 371 Date: Sep. 15, 2011

(30) Foreign Application Priority Data
Mar. 16, 2009 (JP) 2009-062350

(51) Int. Cl. A01H 1/00; A61K 38/47; C12N 5/10; A61P 31/04; A01H 5/00; A61K 35/66; A61P 31/10; A01H 5/10; A61K 35/74; C12N 15/82

(52) U.S. Cl. 800/278; 424/93.2; 424/94.61; 435/419; 435/320.1; 800/298

(57) ABSTRACT

This invention provides a method for preventing or inhibiting infection with a plant-infecting microorganism and imparting resistivity to plants, a method for preparing plants having resistance to diseases caused by microorganisms such as plant pathogenic filamentous fungi, and a microbial pesticide formulation. The method for preventing or inhibiting infection of a host plant with plant-infecting microorganisms comprises degrading α-1,3-glucan on cell walls of the microorganisms by α-1,3-glucanase.

### TECHNICAL FIELD

The present invention relates to a method for preventing or inhibiting infection of plants with plant-infecting microorganisms, a method for producing plants exhibiting resistance to microbial infection, and a microbial pesticide formulation.

### BACKGROUND ART

A cell wall component is a substance that is first recognized by the early immune systems of animals or plants to eukaryotic microorganisms. Animal or plant cells recognize the cell-wall components of eukaryotic microorganisms as microbe-associated molecular patterns (MAMPs), elicit defense mechanisms, and block the infection of the microorganisms. It is known that cell wall chitin, β-glucan, and mannan are recognized as MAMPs in animal cells and chitin and β-glucan are recognized as MAMPs in plant cells (Hogan, L. H., Klein, B. S., Levitz, S. M., 1996, Virulence factors of medically important fungi. Clin. Microbiol. Rev., 9, 469-488; Brown, G. D., Gordon, S., 2005, Immune recognition of fungal β-glucans, Cell, Microbiol., 7, 471-479; Reese, T. A., Liang, H.-E., Tager, A. M., Luster, A. D., Rooijen, N. V., Voehringer, D., Locksley, R. M., 2007, Chitin induces accumulation in tissue of innate immune cells associated with allergy, Nature 447, 92-96; and Altenbach, D., Robatzek, S., 2007, Pattern recognition receptors: From the cell surface to intracellular dynamics, Mol. Plant-Microbe. Interact., 20, 1031-1039).

When plant cells recognize MAMPs, plant cells elicit defense mechanisms, such as production of lytic enzymes (e.g., cell-wall degrading enzymes) or antimicrobial agents, and plant cells block infection with pathogenic organisms (Altenbach, D., Robatzek, S., 2007, Pattern recognition receptors: From the cell surface to intracellular dynamics, Mol. Plant-Microbe. Interact., 20, 1031-1039).

Except for some pathogenic organisms, methods for avoiding the host immune recognition by pathogens in order to deal with cellular defense responses have not been found. It has been found in recent years that a cell wall surface of Histoplasma capsulatum, which is an animal pathogen, is covered with α-1,3-glucan at the time of infection, and chitin on a cell wall surface of Puccinia graminis, Uromyces fabae, or Colletotrichum graminicola, which is a plant pathogen, is converted into chitosan at the time of infection. Such microorganisms are considered to reconstruct cell wall surfaces into components that are less likely to be recognized by host cells, and thus they block host cells from recognizing MAMPs (Rappleye, C. A., Groppe Eissenberg, L., Goldman, W. E., 2007, Histoplasma capsulatum α-(1,3)-glucan blocks innate immune recognition by the β-glucan receptor, Proc. Natl. Acad. Sci., U.S.A., 104, 1366-1370; Eddine El Gueddari, N., Rauchhaus, U., Moerschbacher, B. M., Deising, H. B., 2002, Developmentally regulated conversion of surface-exposed chitin to chitosan in cell walls of plant pathogenic fungi, New Phytologist., 156, 103-112).

The rice blast fungus (Magnaporthe grisea, M. oryzae) are major plant pathogenic filamentous fungi that mainly infect gramineous cereals. Rice is known to be capable of recognizing a chitin oligomer derived from fungal cell walls via a receptor (Kaku, H., Nishizawa, Y., Ishii-Minami, N., Akimoto-Tomiyama, C., Dohmae, N., Takio, K., Minami, E., Shibuya, N., 2006, Plant cells recognize chitin fragments for defense signaling through a plasma membrane receptor, Proc. Natl. Acad., Sci., U.S.A., 103, 11086-11091). In addition to the fact that the avoidance mechanism of the rice blast fungus against recognition of cell-wall chitin by rice is not known, cell wall components at the time of infection with rice blast fungus are not at all known. According to rice genome information (http://www.nias.go.jp), it is apparent that rice does not have α-1,3-glucanase or chitosanase but has β-1,3-glucanase and chitinase. Specifically, it has been strongly deduced that chitin and β-1,3-glucan degradation products are recognized as cell-wall-derived MAMPs of infectious microorganisms in rice and β-1,3-glucanase and chitinase attack microorganisms that have infected cells.

While the cell wall of the rice blast fungus is known to contain heteropolysaccharides having α-bonds (Nakajima et al., the Journal of the Phytopathological Society of Japan, vol. 34, No. 5, December 1968, right column, page 360 (12); Tanaka et al., Ann. Phytopath. Soc. Japan, XXXV (2), March 1969, p. 95, left column (9); Nakajima et al., Ann. Phytopath. Soc. Japan, XXXVI (3), June 1970, p. 159, left column (9); and Nakajima et al., J. Biochem., December 1977; 82 (6):1657-62), specific types or localization thereof are not known.

U.S. Pat. No. 5,670,706 discloses that fungal disease resistance in plants is improved via expression of intracellular chitinase. In addition to the chitinase gene, it also describes that the introduction of the β-1,3-glucanase gene. However, this patent document does not describe that the expression of the β-1,3-glucanase gene alone. In addition, utilization of α-1,3-glucanase is not described at all.

JP Patent Re-publication (Saikohyo) No. WO 98/58065 and JP Patent Re-publication (Saikohyo) No. WO 97/22242 disclose that the introduction of DNA encoding a glucan elicitor receptor into a plant alone or together with the glucanase gene to impart mold resistance to the plant. It is disclosed that when glucanase used therein is expressed alone, however, the resulting resistance is insufficient.

### SUMMARY OF THE INVENTION

The present invention provides a method for preventing or inhibiting infection with a plant-infecting microorganism and imparting resistance to a host plant, a method for preparing a plant having resistance to infection with a plant-infecting microorganism, and a microbial pesticide formulation.

Based on the fact that many plant-infecting microorganisms contain α-1,3-glucan as a constitutive cell wall component and that hyphae and infection structures of some plant-infecting microorganisms are covered with an α-1,3-glucan layer when infecting host cells, the present inventors discovered that the infectivity of the plant-infecting microorganisms inoculated into host plants could be attenuated by degrading such α-1,3-glucan with α-1,3-glucanase. The present invention has been completed based on such finding and provides the following.

(1) A method for preventing or inhibiting infection of a host plant with a plant-infecting microorganism comprising degrading α-1,3-glucan on the microbial cell wall with α-1,3-glucanase.

(2) The method according to (1), wherein the plant-infecting microorganism comprises α-1,3-glucan as a constitutive cell wall component.

(3) The method according to (1) or (2), wherein the plant-infecting microorganism forms a cell-wall-coating layer comprising α-1,3-glucan in response to the contact with a host plant.

(4) The method according to (2), wherein the plant-infecting microorganism is selected from the group consisting of genera Botrytis, Aspergillus, Sclerotinia, Puccinia, Colletotrichum, Fusarium, Alternaria, Rhizoctonia, Sclerotium, Peronospora, Sphaerotheca, and Erysiphe.

(5) The method according to (3), wherein the plant-infecting microorganism is of genera Magnaporthe or Colletotrichum.

(6) The method according to any of (1) to (5), wherein the plant is a dicotyledonous or monocotyledonous plant.

(7) The method according to (6), wherein the plant is a gramineous or solanaceous plant.

(8) The method according to any of (1) to (7), wherein α-1,3-glucan on the cell wall of the plant-infecting microorganism is degraded by α-1,3-glucanase expressed by a foreign gene in the plant.

(9) The method according to any of (1) to (8), wherein α-1,3-glucanase is brought into contact with the plant.

(10) The method according to any of (1) to (9), wherein a microbial pesticide formulation comprising, as an active ingredient, a microorganism that has the α-1,3-glucanase gene and secretes α-1,3-glucanase to the outside of the cell is allowed to act on the plant.

(11) The method according to (10), wherein the α-1,3-glucanase expression level in the microorganism is significantly higher than that of a wild type thereof at the time of normal growth.

(12) The method according to (11), wherein the microorganism is subjected to induction of α-1,3-glucanase expression.

(13) The method according to (12), wherein the induction of expression is addition of α-1,3-glucan.

(14) The method according to any of (10) to (13), wherein the α-1,3-glucanase gene is an endogenous gene.

(15) The method according to (14), wherein the microorganism is of genera Bacillus, Paenibacillus, Aspergillus, and/or Trichoderma.

(16) A method for preparing a plant exhibiting resistance to microbial infection comprising a step of transforming a plant with an expression vector comprising a gene encoding α-1,3-glucanase.

(17) An expression vector comprising a gene encoding α-1,3-glucanase used for the method according to (16).

(18) A plant cell containing the expression vector according to (17).

(19) Plant tissue containing the plant cell according to (18).

(20) A plant body containing the plant cell according to (18) or plant tissue according to (19).

(21) A seed obtained from the plant body according to (20).

(22) A microbial pesticide formulation comprising, as an active ingredient, a microorganism that has the α-1,3-glucanase gene and secretes α-1,3-glucanase to the outside of the cell.

(23) The microbial pesticide formulation according to (22), wherein the α-1,3-glucanase expression level in the microorganism is significantly higher than that of a wild type thereof at the time of normal growth.

(24) The microbial pesticide formulation according to (23), wherein the microorganism is subjected to induction of α-1,3-glucanase expression.

(25) The microbial pesticide formulation according to (24), wherein the induction of expression is addition of α-1,3-glucan.

(26) The microbial pesticide formulation according to any of (22) to (25), wherein the α-1,3-glucanase gene is an endogenous gene.

(27) The microbial pesticide formulation according to (26), wherein the microorganism is of genera Paenibacillus, Bacillus, Trichoderma, and/or Aspergillus.

This description includes part or all of the contents as disclosed in the description and/or drawings of Japanese Patent Application No. 2009-062350, which is a priority document of the present application.

### BRIEF DESCRIPTION OF THE DRAWINGS

FIG. 1 shows detection of cell wall components in the infection structures of rice blast fungus. In the figure, panel A1 and panel A2 each show bright-field images 16 hours and 24 hours after inoculation. Panel B1 and panel B2 each show images of stained α-1,3-glucan, panel C1 and panel C2 each show images of stained β-1,3-glucan, panel D1 and panel D2 each show images of stained chitin, panel F2 shows an image of stained chitosan, and panel H2 shows an image of stained mannan. Panel E2 is a bright-field image corresponding to the stained image of chitosan (F2) and panel G2 is a bright-field image corresponding to the stained image of mannan (H2). An upper panel shows an image from 16 hours after inoculation and the middle and lower panels show images from 24 hours after inoculation. C represents a spore (conidium), G represents a germ tube, A represents an appressorium, IF represents infectious hyphae, and each bar in the panels represents 20 μm.

FIG. 2 shows detection of a cell wall component in infectious hyphae of rice blast fungus after treatment with α-1,3-glucanase. In the figure, panel A is a bright-field image, panel B is an image of stained α-1,3-glucan, panel C is an image of stained β-1,3-glucan, and panel D is an image of stained chitin. “A” represents an appressorium, “IF” represents infectious hyphae, and each bar in the panels represents 20 μm.

FIG. 3A shows a strategy for preparing a defective strain via substitution of the α-1,3-glucan synthase gene (MgAGS1) with a marker gene (i.e., the bialaphos resistance gene (the Bar gene)).

FIG. 3B shows substitution of MgAGS1 with the bialaphos resistance gene (the Bar gene) verified by Southern hybridization. As probes, “AGS1-int2 probe” was used in the upper panel and “Bar probe” was used in the lower panel.

FIG. 4 shows the capacity of a wild-type strain and that of the ΔMgAGS1 strain to generate infection structures. The upper panel shows an image showing generation of an infection structure by a wild-type strain (left) and the α-1,3-glucanase-defective strain (the ΔMgAGS1 strain) (right) on a glass cover. The lower panel shows an image of generation of an infection structure of a wild-type strain (left) and that of the ΔMgAGS1 strain (right) on the thermally-treated cells of the onion scale. C represents a spore (conidium), A represents an appressorium, and IF represents an infectious hyphae.

FIG. 5 shows the lowered infectivity of the ΔMgAGS1 strain against rice. The left panel shows a rice leaf into which a wild-type strain has been inoculated and the right panel shows a rice leaf into which the ΔMgAGS1 strain has been inoculated.

FIG. 6 shows the lowered infectivity of the ΔMgAGS1 strain against barley. The left panel shows a barley leaf into which a wild-type strain has been inoculated and the right panel shows a barley leaf into which the ΔMgAGS1 strain has been inoculated.

FIG. 7 shows inhibition of appressorium and infectious hyphae formation by rice blast fungus in rice with the addition of α-1,3-glucanase. The left panel shows an image of rice cells observed 48 hours after inoculation of a wild-type strain and the right panel shows an image of rice cells observed 48 hours after inoculation of a wild-type strain to which α-1,3-glucanase has been added. The bar shown at the bottom-right corner represents 20 μm.

FIG. 8 shows the lowered infectivity of a wild-type strain when α-1,3-glucanase is added. The left panel shows a rice leaf after inoculation of a wild-type strain to which no α-1,3-glucanase has been added and the right panel shows a rice leaf after inoculation of a wild-type strain to which α-1,3-glucanase has been added.

FIG. 9 shows the lowered infectivity of a wild-type strain when α-1,3-glucanase is added. The left panel shows a barley leaf after inoculation of a wild-type strain to which no α-1,3-glucanase has been added and the right panel shows a barley leaf after inoculation of a wild-type strain to which α-1,3-glucanase has been added.

FIG. 10 shows the transcript amount of the cell wall component synthase gene of rice blast fungus on a plastic surface. Panel (A) shows a microscopic image of spores at the time indicated. The bar shown at the bottom-right corner represents 20 μm. Panel (B) shows the amount of the α-1,3-glucan synthase (MgAGS1) gene and panel (C) shows the transcript amount of the β-1,3-glucan synthase (MgFKS1) gene relative to the transcript amount of the actin gene.

FIG. 11 shows the transcript amounts of the cell wall component synthase genes by rice blast fungus in rice. Panel (A) shows the transcript amount of MgAGS1 and panel (B) shows the transcript amount of MgFKS1 relative to the transcript amount of the actin gene.

FIG. 12 shows an example of the expression vector according to the present invention.

FIG. 13 shows an example of the expression vector according to the present invention.

FIG. 14 shows an example of the expression vector according to the present invention.

FIG. 15 shows an example of the expression vector according to the present invention.

FIG. 16A shows the results of gel electrophoresis confirming the incorporation of the AGL gene into genomic DNA of the T0 transgenic rice plants containing the AGL gene.

FIG. 16B shows the results of observation of AGL gene expression in the T0 transgenic rice plants containing the AGL gene via RT-PCR.

FIG. 16C shows the results of observation of the Agl protein in the T0 transgenic rice plants containing the AGL gene via western blot analysis.

FIG. 17 shows resistance of the T0 transgenic rice plants containing the AGL gene to compatible rice blast fungus.

FIG. 18 shows resistance of the T0 transgenic rice plants containing the AGL gene to incompatible rice blast fungus.

FIG. 19 shows resistance of the T0 transgenic rice plants containing the AGL gene to Cochliobolus miyabeanus.

FIG. 20 shows AGL gene expression in the T1 transgenic rice plants containing the eAGL gene.

FIG. 21 shows resistance of the T1 transgenic rice plants containing the AGL gene to rice blast fungus.

FIG. 22 shows resistance of the T1 transgenic rice plants containing the AGL gene to Cochliobolus miyabeanus.

FIG. 23A shows resistance of the T1 transgenic rice plants containing the AGL gene to Thanatephorus cucumeris.

FIG. 23B shows resistance of the T1 transgenic rice plants containing the AGL gene to Thanatephorus cucumeris.

FIG. 24A shows inhibition of tobacco leaf infection with Botrytis cinerea treated with α-1,3-glucanase. “a” represents tobacco leaf into which Botrytis cinerea spores treated with 1,3-glucanase had been inoculated, and “b” represents tobacco leaf into which Botrytis cinerea spores suspended in a buffer containing no 1,3-glucanase had been inoculated.

FIG. 24B shows inhibition of infection with Botrytis cinerea via transient expression of α-1,3-glucanase in tobacco leaf. In “a”, the region surrounded by broken line represents a region into which gray mold had been inoculated at a site in which 1,3-glucanase had been transiently expressed, and in “b”, a region surrounded by broken line represents a region into which gray mold had been inoculated at a position in which no 1,3-glucanase is to be expressed.

FIG. 25 shows expression of the AGL gene in the Bacillus circulans KA304 strain secreting α-1,3-glucanase.

FIG. 26 shows effects of protection of infection of rice with rice blast fungus via inoculation with the Bacillus circulans KA304 strain, which is an active ingredient of the microbial pesticide formulation.

FIG. 27 shows α-1,3-glucan on the cell walls of plant-infecting microorganisms that had infected rice plants (Nipponbare N2). BF in the left panel shows a bright field image and α-G in the right panel shows antibody staining of α-1,3-glucan.

FIG. 28 shows α-1,3-glucan on the cell walls of a variety of plant-infecting microorganisms. BF shows a bright field image and α-G shows antibody staining of α-1,3-glucan.

### EMBODIMENTS FOR CARRYING OUT THE INVENTION

1. Method for Preventing or Inhibiting Infection with Plant-Infecting Microorganism

### 1-1. Constitution

The first embodiment of the present invention relates to a method for preventing or inhibiting infection with plant-infecting microorganisms in a host plant. The method for preventing or inhibiting infection with plant-infecting microorganisms according to the present invention comprises degrading α-1,3-glucan on the cell wall of the plant-infecting microorganism with α-1,3-glucanase.

In the present invention, the term “microorganisms” refers to organisms of a size that makes them difficult to visually recognize, i.e., unicellular eukaryotic microorganisms, such as yeast, and multicellular eukaryotic microorganisms that are difficult or possible to visually recognize, such as filamentous fungi (including molds) orbasidiomycetes (e.g., mushrooms).

The term “plant-infecting microorganisms” refers to microorganisms that can infect plants and cause certain pathological symptoms in host cells upon infection therewith. The target plant-infecting microorganisms of the present invention are required to have α-1,3-glucan at least on the cell wall. α-1,3-glucan on the cell wall may be a constitutive cell wall component, or it may be contained in a cell-wall-coating layer formed upon contact with a host plant. The expression “upon contact with a host plant” refers to a condition in which, when plant-infecting microorganisms or spores thereof are brought into contact with a host cell, plant-infecting microorganisms or spores thereof recognize the hardness of a host plant surface or a wax on the plant surface, and they react with such substances, for example.

Hereafter, specific examples of the target plant-infecting microorganisms of the present invention are provided. It should be noted that the names of diseases provided below are merely the names of diseases caused by the microorganisms. For example, various other names listed in Table 1 are within the scope thereof. Accordingly, the method for preventing or inhibiting infection with plant-infecting microorganisms according to the present invention can be regarded as a system for preventing diseases specified by the disease names provided below.

Representative examples of plant-infecting filamentous fungi having α-1,3-glucan as a constitutive cell wall component include Botrytis fungi (genus Botryotinia) such as Botrytis cinerea, Aspergillus fungi (genus Eurotium) such as Aspergillus flavus (opportunistic infection: aflatoxin-producing fungi), Colletotrichum fungi (genus Glomerella) such as Colletotrichum acutatum and Colletotrichum orbiculare, Fusarium fungi (the genera Gibberella, Haematonectoria, Nectoria, and Calonectoria) such as Fusarium oxysporum, Alternaria fungi such as Alternaria alternata or Alternaria solani, Rhizoctonia fungi (genus Thanatephorus) such as Rhizoctonia solani, and Sclerotium fungi such as Sclerotium rolfsii.

Damage caused by basidiomycetes (i.e., mushrooms) is generally problematic for fruit trees; however, many mushroom species are considered to have α-1,3-glucan on their cell walls. Specific examples include fungi of the genus Sclerotinia (e.g., Sclerotinia sclerotiorum), and fungi of the genus Puccinia (e.g., genus Aecidium) such as Puccinia recondita (Puccinia allii) of Allium.

In particular, fungi of the genus Botrytis, fungi of the genus Aspergillus such as Aspergillus niger and Aspergillus flavus, and fungi of the genera Sclerotinia, Puccinia, Colletotrichum, Fusarium, Rhizoctonia, and Sclerotium are plurivorous, such fungi impose serious damages on various crops, and thus are important plant-infecting fungi.

Representative examples of plant-infecting filamentous fungi containing α-1,3-glucan on the cell-wall-coating layer formed upon contact with a host plant include fungi of the genera Magnaporthe and Colletotrichum.

Other examples of major plant-infecting microorganisms (including Ascomycetes, Basidiomycetes, and Oomycetes) include Taphrina (e.g., Taphrina deformans), Blumeria (e.g., Blumeria graminis (Erysiphe graminis)), Cystotheca (e.g., Cystotheca wrightii), Erysiphe (e.g., Erysiphe pulchra (Microsphaera pulchra)), Golovinomyces (e.g., Golovinomyces cichoracearum (Erysiphe cichoracearum)), Phyllactinia (Ovulariopsis) (e.g., Phyllactinia moricola), Podosphaera (Sphaerotheca) (e.g., Podosphaera tridactyla), Sawadaea (Oidium) (e.g., Sawadaea polyfida), Ceratocystis (e.g., Ceratosystis fimbriata), Monosporascus (e.g., Monosporascus cannonballus), Claviceps (Ustilaginoidea and Sphaecelia) (e.g., Claviveps virens (Ustilaginoidea virens)), Calonectria (Cylindrocladium) (e.g., Calonectria ilicicola (Cylindrocladium parasiticum)), Gibberella (e.g., Gibberella fujikuroi and Gibberella zeae), Haematonecria (e.g., Haematonecria haematococca (Fusarium solani)), Nectria (e.g., Nectria cinnabarina (Tubercularia vulgaris)), Neonectria (e.g., Neonectria castaneicola (Cylindrocarpon castaneicola)), Glomerella (e.g., Glomerella cingulata (Colletotrichum gloeosporioides)), Cryphonectria (e.g., Cryphonectria parasitica (Endothiella parasitica)), Diaporthe (e.g., Diaporthe tanakae (Phomopsis sp.)), Valsa (e.g., Valsa ceratosperma (Cytospora rosarum)), Pestalosphaeria (e.g., Pestalosphaeria gubae (Pestalotiopsis neglecta)), Rosellinia (e.g., Rosellinia necatrix), Ciborinia (e.g., Ciborinia camelliae), Ovulinia (e.g., Ovulinia azaleae), Monilinia (e.g., Monilinia fructicola), Diplocarpon (e.g., Diplocarpon rosae (Marssonina rosae)), Elsinoe (e.g., Elsinoe fawcetti (Sphaceloma citri)), Cochliobolus (e.g., Cochliobolus heterostrophus (Bipolaris maydis) and Cochliobolus miyabeanus (Bipolaris oryzae)), Didymella (e.g., Didymella bryoniae (Ascochyta cucumis)), Pleospora (e.g., Pleospora herbarum (Stemphylium sp.)), Venturia (e.g., Venturia nashicola), Mycosphaerella (e.g., Mycosphaerella chaenomelis (Cercosporella chaenomelis)), Helicobasidium (e.g., Helicobasidium mompa), Ustilago (e.g., Ustilago maydis), Tilletia (e.g., Tilletia caries), Exobasidium (e.g., Exobasidium japonicum), Coleosporium (e.g., Coleosporium pini-asteris), Cronartium (e.g., Cronartium orientale), Melampsora (e.g., Melampsora hypericorum), Phakopsora (e.g., Phakopsora euvitis), Phragmidium (e.g., Phragmidium montivagum), Gymnosporangium (e.g., Gymnosporangium asiaticum), Uromyces (e.g., Uromyces viciae-fabae), Blastospora (e.g., Blastospora smilacis), Thanatephorus (e.g., Thanatephorus cucumeris (Rhizoctonia solani)), Armillaria (e.g., Armillaria mellea), Erythricium (e.g., Erythricium salmonicolor), Perenniporia (e.g., Perenniporia fraxinea), Ganoderma (e.g., Ganoderma applanatum), Phoma (e.g., Phoma exigua), Pyrenochaeta (e.g., Purenochaeta lycopersici), Phomopsis (e.g., Phomopsis asparagi), Gloeodes (e.g., Gloeodes pomigena), Tubakia (e.g., Tubakia japonica), Ascochyta (e.g., Ascochyta aquilegiae), Lasiodiplodia (e.g., Lasiodiplodia theobromae), Pestalotiopsis (e.g., Pestalotiopsis maculans), Ateroconium (e.g., Asteroconium saccardoi), Oidiopsis (e.g., Oidiopsis sicula), Verticillium (e.g., Verticillium dahliae), Penicillium (e.g., Penicillium italicum), Cladosporium (e.g., Cladosporium paeoniae), Corynespora (e.g., Corunespora cassiicola), Fulvia (e.g., Fulvia fulva), Cercospora (e.g., Cercospora apii)), Pseudocercospora (e.g., Pseudocercospora egenula (Pracercospora egenula)), Aphanomyces (e.g., Aphanomyces raphani), Phytophthora (e.g., Phytophthora cactorum and Phytophthora infestans), Pythium (e.g., Pythium irregulars), Albugo (e.g., Albugo macrospore), Peronospora (e.g., Peronospora parasitica), Plasmopara (e.g., Plasmopara viticola), Rhizopus (e.g., Rhizopus stolonifer), and Choanephora (e.g., Choanephora cucurbitarum).

Examples of major plant-infecting bacteria comprising α-1,3-glucan as a constitutive cell wall component include Xanthomonas bacteria such as Xanthomonas oryzae pv. oryzae, Xanthomonas axonopodis pv. malvacearum, Xanthomonas theicola, and Xanthomonas axonopodis pv. citri, Pseudomonas bacteria such as Pseudomonas savastanoi pv. phaseolicola, Pseudomonas savastanoi pv. glycinea, and Pseudomonas syringae pv. tomato, Ralstonia bacteria such as Ralstonia solanacearum, Acidovorax bacteria such as Acidovorax avenae subsp. avenae, Burkholderia bacteria such as Burkholderia glumae, Erwinia bacteria (including Pectobacterium and Dickeya) such as Erwinia carotovora subsp. wasabiae and Pectobacterium carotovorum (=syn. Erwinia carotovora), Pantoea bacteria such as Pantoea ananas pv. ananas, Agrobacterium bacteria (including Rhizobacter) such as Agrobacterium rhizogenes, Clavibacter bacteria such as Clavibacter michiganensis subsp. michiganensis, Corynebacterium bacteria such as Corynebacterium sp. and Corynebacterium michiganense pv. sepedonicum, Streptomyces bacteria such as Streptomyces sp., Microbacterium bacteria such as Microbacterium sp., Xylella bacteria such as Xylella fastidiosa, and Clostridium bacteria, such as Clostridium sp.

TABLE 1

| Pathogenic organisms | Diseases |
|---|---|
| Botrytis | Gray mold, red spot disease, stem canker, Tate-boya |
| (=Botryotinia) | disease, storage rot, barn rot, leaf blight, brown leaf spot, |
| | sclerotical rot, damping-off, spot plant disease, brown |
| | blight, small sclerotical rot, brown spot plant disease, small |
| | sclerotical blight, blight, hyphae rot |
| Rosellinia | White root-rot, Rosellinia needle blight, black rot, root rot |
| | of seedlings, root rot |
| Aspergillus | Black mold, Aspergillus blight, bulb black mold, |
| (=Eurotium etc.) | yellow-spotted rice, dark colorsis of rice grains, clouded |
| | rice grain, white koji mold-infected rice, Nicaraguan |
| | browned rice, rice infected with Aspergillus versicolor |
| Phytophthora | blight, rot, wilt, Ink disease, Phytophthora canker, |
| | Phytophthora root rot, downy mildew, blight, yellow |
| | dwarf, bud rot, gray blight, brown rot, brown spot, |
| | stemrot, blackline disease, root rot, little leaf, heart rot, |
| | black shank of tobacco, red canker, barn rot, soft rot, blight |
| | and rot, pink rot, seedling blight, damping-off of seedlings, |
| | rot, cotton blight, leaf blight, damping off blight |
| Pythium | rhizome rot, root rot, rot, snow mold |
| Sclerotinia | Sclerotical disease, stem rot, Ezo-raigan disease, |
| | rust-rolored Sclerotiose, yellow rot, gray sclerotical |
| | disease, brown sclerotical root rot, brown sclerotical |
| | disease, Typhula incarnata disease, Cristulariella moricola |
| | disease, Sclerotium hydrophilum disease, leaf smut, black |
| | bulb disease, black rot sclerotical disease, dieback |
| | sclerotical disease, damping-off of sprout, small sclerotical |
| | disease, southern blight, black root rot, crown rot, sheath |
| | blight |
| Colletotrichum | Anthracnose, treetop wilting, black stain (melanose-type) |
| (=Glomerella) | disease, Glomerella cingulata disease, fruit rot, stem-end |
| | rot |
| Rhizoctonia | web blight, blister rust, black end, sooty blight, bottom rot, |
| (=Thanatephorus) | shot-hole leaf blight, dollar spot, Rhizina root rot, stem-end |
| | rot, Rhabdocline needle cast, Rhizoctonia root rot, |
| | Rhizoctonia disease, leaf sheath rot, Rhizoctonia |
| | damping-off, dark snow mold, wilting, scald, leaf spot, bud |
| | rot, kaiware disease, gray sclerotical disease, gray rot, |
| | brown scab, brown ring leaf rot, brown sclerotical disease, |
| | brown spot plant disease, brown sheath blight disease, |
| | brown spot root rot, brown spot disease, foot rot disease, |
| | dry root rot, deforming symptons, yellow patch, bulb rot, |
| | chestnut root rot, stem rot, banded sclerotial disease, black |
| | scab, black mold, black root rot, melanose, black spot |
| | disease, black leaf rot disease, crown canker, root rot |
| | symptoms, root rot decline, rot, cracks and browning of the |
| | root, soft rot of rootlets, dieback, fruit rot, black dot |
| | disease, bottom rot, root rot of hydroponic seedlings, |
| | reddish-brown leaf rot, red sclerotical disease, red blight, |
| | red ring disease, fasciation, sheath blight, charcoal rot, barn |
| | rot, soft rot, white leaf rot, bulb-coat rot, fine sclerotical |
| | disease, seedling blight, seedling rot, damping-off of |
| | seedlings, rot, sheath blight disease, leaf blotch, leaf spot, |
| | leaf rot, damping-off, woodland root rot, ring spot, seedpod |
| | brown spot disease, seedpod rot, black patch |
| Helicobasidium | Violet root rot |
| Fusarium | Bakanae disease, fusarium head blight, wilting, root rot, |
| (=Gibberella, | damping-off, blight, brown spot, damping-off of seedlings, |
| Haematonectoria, | rot, canker, treetop wilting, top rot, black end, Fusarium rot |
| Nectoria, and | of bamboo-shoot, Fusarium wilt, Panama disease, Fusarium |
| Calonectoria) | patch, Fusarium wilting, Fusarium fruit rot, Fusarium |
| | disease, Fusarium rot, Fusarium damping-off, twig blight, |
| | yellow spot, peduncle rot, fruit rot, bud rot, brown dry rot, |
| | brown rot, brown blight, wilt, foot rot disease, dry rot, cone |
| | browning, bulb rot, northern anthracnose, stem blight, stem |
| | rot, cone rot, pink snow mold, pink rot, black scab, black |
| | smear, black stripe fruit rot, root constriction, crown rot, |
| | root rot withers, root rot decline, damping-off of young |
| | seedlings, dieback, stem-end rot, fruit rot, seed rice rot, |
| | seedling root rot, heart rot, water spot, fusarium |
| | damping-off, stalk blight, snow mold, tipburn, stem canker, |
| | pitch canker, scab, Fusarium blight, spot plant disease, leaf |
| | sheath rot, damage by continuous cropping, ring spot, |
| | resinous stem canker |
| Magnaporthe | Blast, stem rot |
| (=Pyricularia) | |
| Alternaria | Alternaria stem blight, Alternaria spot plant disease, |
| | Alternaria blight, bract and leaf spot, black scab on pod, |
| | sooty spot, sooty mold, pseudo-blast, pseudo-black spot, |
| | sooty Blotch, bract blight, fruit stain disease, early-blight, |
| | fruit black spot, fruit disease, bud rot, bud rot, gray spot |
| | plant disease, brown mottle, browned rice, brown spot |
| | disease, brown spot, brown leaf spot, cone browning, black |
| | mold, black mold, Alternaria sooty spot, black spot, black |
| | spot, black rot, kernel smudge, black blight, seed spot plant |
| | disease, neck rot, melanose-like blemish, heart rot, |
| | chocolate-like blemish, barn rot, white rust, white leaf spot, |
| | spot plant disease, leaf stripe, leaf stripe, damping-off of |
| | seedlings, kernel discoloration, leaf curl disease, leaf |
| | blight, damping-off, ring spot |
| Puccinia | Rust, yellow rust, brown rust, brown circular leaf spot, |
| (=Aecidium) | crown rust, black rust, dwarf leaf rust, orange rust, pink |
| | disease, southern rust, white rust, leaf rust |
| Uromyces | Rust, stem rust, dwarf leaf rust |
| Microsphaera | Powdery mildew |
| Blumeria | |
| Erysiphe | |
| (=Golovinomyces) | |
| Oidium | |
| (=Sawadae) | |
| Sphaerotheca | Powdery mildew, violet powdery mildew |
| (=Podosphaera) | |
| Phyllactinia | Powdery mildew |
| (=Ovulariopsis) | |
| Uncinula | |
| (=Golovinomyces, | |
| Erysiphe) | |
| Podosphaera | Powdery mildew |
| (=Sphaerotheca) | |
| Pythium | Wilting, brown snow mold, foot rot disease, stem blight, |
| | stem and root rot, root rot, root rot withers, brown-blotted |
| | root rot, stunting, Pythium root rot, Pythium, Pythium |
| | damping-off of seedlings, Pythium rot, Pythium blight, |
| | Pythium damping-off, browning root rot, fruit rot, stem rot, |
| | Pythium stalk rot, rhizome rot, root rot decline, soft rot of |
| | rootlets, dieback, pustule, root rot of hydroponic seedlings, |
| | stalk blight, shoot blight, white rot, black rot of seedlings, |
| | root rot of seedlings, seedling rot, damping-off of |
| | seedlings, rot, bed rot, cottony lead, root injury by |
| | continuous cropping at wood land exhibiting damping off |
| Peronospora | Downy mildew |
| Ustilago | Smut, leaf smut, gall smut, stripe smut, covered smut, grain |
| | smut, loose smut, grain smut, kernel smut |
| Cochliobolus | brown spot, leaf spot, sooty blight, pseudo-blast, |
| (=Curvularia, Bipolaris, | Helminthosporium blight, Fusicoccum leaf blight, summer |
| Helminthosporium) | leaf spot, brown stripe, brown blight, blackpoint and kernel |
| | smudge, spot plant disease, Northern spot plant disease, |
| | leaf blight |
| Cladosporium | Sooty mold, Northern leaf blight, black spot, common scab, |
| (=Mycosphaerella, | leaf scab, pseudo-blast, fruit stain disease, leaf mold, gray |
| Venturia) | leaf spot, stem speckle, brown spot disease, dry rot, black |
| | pustules, black rot, cereal mould, heart rot, spot plant |
| | disease, brown stem rot, leaf stripe, leaf mold, black spot |
| | (kokuryuutennbyo), leaf blight, tipburn, leaf blotch, leaf |
| | spot, Cladosporium disease (urahai byo) |
| Mycosphaerella | Brown spot disease, fruit spot, melanose, black spot, |
| (=Ascochyta, Cercospora, | mottle, heart rot, leaf spot, sooty mold, canker, Septoria |
| Phyllosticta, Septoria) | stem canker, shot-hole brown spot, short hole, Capsicum |
| | spot, canker, pseudo-defoliation, leaf blotch, circular leaf |
| | spot, Mycosphaerella mawae disease, circular leaf spot, |
| | yellow spot, bud rot, gray spot plant disease, gray blight, |
| | brown short hole, brown leaf spot, black smear, black leaf |
| | blight, black powdery spot, scab, dieback, purple-rimmed |
| | gray leaf spot, white powdery spot, white mold, white leaf |
| | blight, frosty mildew, leaf stripe, rot, leaf cast, blight, |
| | pustule, tipburn, leaf blotch, defoliation, damping-off |
| Phomopsis | Stem canker, canker, Fusarium wilt, Phomopsis rot, stem |
| (=Diaporthe) | blight, Phacidiopycnis stem canker, Phomopsis stem |
| | canker, bud blight, Phomopsis root rot, Phomopsis |
| | dieback, Phomopsis damping-off, fruit stain disease, fruit |
| | soft rot, fruit rot, gray blight, stem speckle, brown stripe, |
| | brown leaf spot, dry rot, concave rot, melanose, stem-end |
| | rot, dieback, branch spot disease, swelling arm, stem-end |
| | rot, fruit dry rot, gummosis, bottom rot, needle leaf |
| | melanose, burned tip, berry drop, spot plant disease, leaf |
| | stripe, collar rot, tipburn |
| Phoma | brown spot, wilt, canker, seed pod pustule, leaf spot, |
| (=Leptosphaeria, | bottom rot, phoma dieback, canker, glume blight, |
| Mycosphaerella) | fire-blight, circular leaf spot, circular leaf spot, stem |
| | speckle, browned rice, brown ring spot, brown leaf spot, |
| | dryblack rot, pseudo-stem canker, stem blight, black rot, |
| | black root rot, black spot, melanose, black spot, black rot, |
| | Phoma stem rot, root black spot, root rot, gangrene, |
| | dieback, branch blight, stem-end rot, fruit spot plant |
| | disease, fruit rot, leaf spot, heart rot, shoot dwarf, tipburn, |
| | early rot, storage rot, stem canker, feline eye disease, white |
| | leaf blight, white spot disease, spot plant disease, leaf |
| | stripe, damping-off of seedlings, cluster rot, streak, |
| | blackleg, blight, tipburn, defoliation, damping-off, ring |
| | spot |
| Venturia | Black spot, canker, spring defoliation, purpura |
| (=Fusicladium, | |
| Spilocaea) | |
| Claviceps | False smut of rice, ergot disease |
| (=Ustilaginoidea, | |
| Sphaecelia) | |
| Calonectria | Brown blight, brown spot, stem blight, blackleg, black root |
| (=Cylindrocladium) | rot, pink snow mold, stem blight, green point gall |
| Verticillium | Blue stem, wilting, Verticillium melanose disease, |
| (=Cosmospora, | Verticillium wilting, Verticillium disease, dwarf, icterus, |
| Hypocereales, etc.) | brown spot, cereal mould, root rot, gray leaf spot, |
| | damping-off |
| Penicillium | Blue mold, damping-off of seedlings, Penicillium rot, |
| (=Eupenicillium, | Islandia yellowed rice, Thailand yellowed rice, rice |
| Talaromyces) | infected with Penicillium cyclopium, rice infected with |
| | Penicillium commune, rice infected with Penicillium |
| | rugulosum, yellowed rice, discolorated fruit core, brown |
| | circular leaf spot, blue mold root rot, blue-green mold, |
| | damping-off, green mold |
| Elsinoe | Scab, common scab, leaf spot, anthracnose, bud atrophy, |
| (=Sphaceloma) | white leaf blight |
| Didymella | Canker, yellow spot, brown spot, canker, circular leaf spot, |
| (=Ascochyta, Phoma, etc.) | large ring spot, spot plant disease |

α-1,3-glucan does not exist on the cell walls of host plants of the aforementioned microorganisms. Accordingly, there may be no or substantially no unfavorable influences, such that a plant cell wall is damaged by contact with α-1,3-glucanase. In the present invention, accordingly, the range of host plants (infected plants) to be protected (i.e., to be prevented or inhibited from infection with plant-infecting microorganisms) is very extensive. Examples thereof include mosses, ferns, angiosperms, and gymnosperms. In the present invention, angiosperms may be dicotyledonous or monocotyledonous plants. Representative examples include agriculturally or commercially important plants, such as crop plants, including grain crops, flowers, vegetables, and fruits.

Specific examples of monocotyledonous plants include rice, wheat, barley, rye, oat grass, Coix lacryma-jobi, millet, Setaria italica, Echinochloa esculenta, Eleusine coracana, maize, Sorghum bicolor, kaoliang, sorghum, sugar cane, bamboo, bamboo grass, Zizania latifolia, Miscanthus sinensis, reed, Zoysia, ginger, Zingiber mioga, Avena sativa, and rye. Specific examples of dicotyledonous plants include solanaceous plants (e.g., tobacco, tomato, eggplant, cucumber, pimento, Capsicum, and Petunia), Leguminosae plants (e.g., bush bean, soy bean, peanut, lentils, garden pea, horse bean, Vigna unguiculata, kudzu, sweet pea, and tamarind), Rosaceae plants (e.g., strawberry, rose, Japanese plum, cherry, apple, Pyrus pyrifolia pear, peach, loquat, almond, plum, quince, hawthorn, Chaenomelis fructus, and kerria), Cucurbitaceae plants (e.g., cucumber, gourd, pumpkin, melon, water melon, and dishcloth gourd), Liliaceae plants (e.g., lily, green onion, and onion), Brassicaceae plants (e.g., lettuce, cabbage, Japanese radish, and Chinese cabbage), Vitaceae plants (grape), Rutaceae plants (e.g., mandarin orange, orange, grapefruit, lemon, and Citrus junos), Malvaceae plants (e.g., okra), Primulaceae plants (e.g., Cyclamen), Theaceae plants (e.g., tea), Begoniaceae plants (e.g., Begonia), Moraceae plants (e.g., fig and mulberry), Actinidiaceae plants (e.g., kiwi fruits), Anacardiaceae plants (e.g., pistachio and mango), Piperaceae plants (e.g., pepper), Myristicaceae plants (e.g., nutmeg), and Ericaceae plants (e.g., Rhododendron metternichii, Rhododendron indicum, azalea, and Rhododendron simsii).

The correlation between plant-infecting microorganisms and host plants thereof (i.e., plants that can be hosts for relevant plant-infecting microorganisms) are listed in Table 2. Thus, the method of the present invention is effective at least when the plant-infecting microorganisms listed in Table 2 infect relevant host plants listed in Table 2.

TABLE 2

| Pathogenic organisms | Host plants |
|---|---|
| Colletotrichum sp. | Artichoke, Aucuba japonica, Akebia quinata, anemone, |
| | avocado, Amacrinum, apricot, anthurium, isotoma, |
| | strawberry, poa, fig, Ficus erecta, Ficus elastica, Impatiens, |
| | Japanese plum, lacquer tree, Vitis ficifolia, garden pea, |
| | Avena sativa, Prunus avium, orchardgrass, Rhynchosia |
| | acuminatifolia, olive, Gerbera, maple, Japanese persimmon, |
| | Cattleya, pumpkin, caladium, kiwi fruits, Dianella ensifolia, |
| | cassava, cucumber, Nerium indicum, Antirrhinum, |
| | calendula, Leucaena leucocephala, guava, Castanea |
| | crenata, croton, mulberry, Celosia, poppy, Kentia palm, |
| | pepper, cosmos, wheat, Hoya carnosa, Camellia sasanqua, |
| | Castanopsis, Cyclamen, sisal hemp, meadow sweet, |
| | Rhododendrons, Chrysanthemum coronarium, Cucumis |
| | melo conomon, Neolitsea sericea, Syngonium, |
| | Symphoricarpos, sweet pea, water melon, Statice, stock, |
| | violet, plum, Erythrina crista-galli, pear, celery, Cordyline |
| | terminalis, Jasminum grandiflorum, Pandanus boninensis, |
| | tobacco, Aralia elata, Dahlia, timothy, China grass, azaleas, |
| | Camellia, Dieffenbachia, Erythrina variegata, Dendrobium, |
| | wax gourd, Enkianthus perulatus, maize, tall oatgrass, |
| | Rhynchosia acuminatifolia, Fraxinus japonica, tomato, |
| | dracaena, Eustoma grandiflorum, Pyrus pyrifolia, eggplant, |
| | Stewartia pseudocamellia, Ilex chinensis, Quercus, |
| | Momordica charantia, Madagascar periwinkle, verbena, |
| | Cotinus coggygria, Lotus, Hernandia sonora, paspalum, |
| | parsley, Passiflora edulis, papaya, Crinum, Hevea, alder, |
| | Anona squamosa, Echinochloa esculenta, pitaya, Schima |
| | wallichii, Stemona japonica, loquat, Phalaenopsis, fescue, |
| | Phoenix, grape, rose apples, bluegrass, blueberry, |
| | bromegrass, Begonia, dishcloth gourd, safflower, safflower, |
| | velvet grass, Mahonia fortunei, Pothos, toad lily, oriental |
| | melon, Cydonia oblonga, mango, Hibiscus syriacus, melon, |
| | Ternstroemia gymnanthera, peach, Corchorus olitorius, |
| | Indian mulberry, Cinnamomum japonicum, Dioscorea |
| | japonica, Lagenaria siceraria, Eucalyptus, ryegrass, lilac, |
| | lovegrass, lanzones, apple, gentian, redtop, Sanguisorba |
| | officinalis |
| Rhizoctonia sp. | Ilex macropoda, Picea glehnii, red clover, Acacia, thistle, |
| | Hydrangea, azuki bean, aster, Astilbe, Asteriscus |
| | maritimum, Thujopsis dolabrata, Asparagus officinalis, |
| | Anemone, avocado, flax, iris, Alyssum, alsike clover, |
| | Alternanthera, alfalfa, Setaria italica, Juncus decipiens, |
| | Amorpha fruticosa, strawberry, fig, Ginkgo biloba, cypress, |
| | rice, orris, bush bean, Impatiens, Lonicera gracilipes, Aralia |
| | cordata, Japanese plum, lacquer tree, endive, garden pea, |
| | Avena sativa, Prunus avium, orchardgrass, Swietenia |
| | macrophylla, barley, Salsola komarovii, okra, Otacanthus |
| | caeruleus, columbine, Lamium album, Patrinia |
| | scabiosaefolia, carnation, Gerbera, maple, Gazania, oak, |
| | Gypsophila, Anthocephalus chinensis, turnip, pumpkin, |
| | Viburnum, Calla lily, leaf mustard, larch, Calceolaria, |
| | citrus, Canna, Campanula, birch, Platycodon, |
| | Chrysanthemum morifolium, ivy, Astragalus membranaceus, |
| | millet, cabbage, Brassica rapa var. nipposinica, empress |
| | tree, Antirrhinum, Hypericum patulum, calendula, kudzu, |
| | Guzmania, Gladiolus, Trifolium incarnatum, gloxinia, |
| | Crocus, mulberry, Verocay virginica, Celosia, poppy, Kenaf, |
| | Zelkova, Japanese umbrella pine, coffee, Cocos nucifera, |
| | cosmos, cotoneaster, oriental arbor vitae, burdock, sesame, |
| | Brassica chinensis komatsuna, Japanese hemlock, common |
| | vetch, Japanese white pine, coleus, Colchicum, |
| | Amorphophalus konjak, primrose, sweet potato, Colocasia |
| | esculenta, sugar cane, subterranean clover, Cactus, |
| | Saponaria ocymoides, Salvia, Chamaecyparis pisifera, |
| | Sandersonia, winged bean, Digitalis, Cyclamen, Eleusine |
| | coracana, Cyperus monophyllus, Zinnia, cineraria, Zoysia, |
| | Phlox subulata, meadow sweet, potato, Gypsophila |
| | paniculata, ginger, Cucumis melo conomon, white mustard, |
| | white clover, Phoenix roebelenii, Melilotus alba, sweet |
| | vernal grass, sweet pea, honeysuckle, Lychnis coronaria, |
| | water lily, Cryptomeria japonica, Statice, stevia, Sutera, |
| | stock, Pinus strobus, snowdrop, violet, pear, Achillea |
| | milefolium, Polemonium caeruleum, Geranium, Oenanthe |
| | javanica, celery, Stenotaphrum secundatum, Fagopyrum, |
| | horse bean, Japanese radish, Brassica campestris var. |
| | chinensis, soy bean, Cannabis, Douglas fir, tobacco, onion, |
| | Dahlia, chicory, timothy, tea, asplenium, tulip, ginseng, |
| | China grass, abelia, azaleas, jute, teosinte, Delphinium, |
| | sugarbeet, Fimbristylis dichotoma, Capsicum, wax gourd, |
| | Angelica acutiloba, Enkianthus perulatus, spruce, maize, |
| | tall oatgrass, Abies sachalinensis, Fraxinus japonica, |
| | tomato, Durio zibethinus, Eustoma grandiflorum, trefoil, |
| | butcher's-broom, Pyrus pyrifolia, eggplant, rapeseed, Ilex |
| | chinensis, Quercus, Sapium sebiferum, Thuja, Momordica |
| | charantia, Robonia pseudoacasia, Madagascar periwinkle, |
| | Allium tuberosum, carrot, green onion, Lyonia ovalifolia |
| | ssp. neziki, Juniperus rigida, Cirsium japonicum, yarrow, |
| | verbena, pineapple, Lespedeza, Chinese cabbage, |
| | Amaranthus tricolor, paspalum, Rhus succedanea, parsley, |
| | banana, Eschscholtzia, Panicum, papaya, ornamental |
| | cabbage, Bermuda grass, rose, Artocarpus integra, rosids, |
| | alder, Anona squamosa, Echinochloa esculenta, pitaya, |
| | cypress, Hypericum, Ricinus, Cedrus, sunflower, |
| | Andromeda polifolia, Schima wallichii, juniper, Pimpinella |
| | saxifraga, Bouvardia, fescue, Phoenix, Chinese Hibiscus, |
| | grape, Hypsizygus marmoreus, Primula species, bluegrass, |
| | prune, broccoli, bromegrass, Begonia, Petunia, safflower, |
| | Phaseolus coccineus, Peperomia, hairly vetch, velvet grass, |
| | Pentas, Penstemon (beard tongue), wheatgrass, Euphorbia |
| | pulcherima, Abutilon, spinach, Physalis, Portulaca, |
| | poplars, Ammi majus, oriental melon, Mesembryanthemum, |
| | pines, marigolds, Hamamelis, Cryptotaenia japonica, |
| | Edgeworthia, Gymnaster savatieri, catchfly, Arundinaria, |
| | meadow foxtail, melon, Casuarina cunninghamiana, firs, |
| | peach, Sorghum bicolor, Corchorus olitorius, yacon, |
| | willows, Dictyopteris, Dioscorea japonica, Lagenaria |
| | siceraria, Eucalyptus, Euphorbia, Saxifraga, Euryops, |
| | Liliaceae species, reeds, ryegrass, rye, peanut, Liatris, reed |
| | canary grass, apple, gentian, rutabaga, lupine, Echinops |
| | ritro, Litchi chinensis, Leonotis, leatherleaf fern, lettuce, |
| | redtop, Rhodes grass, horse radish, cotton, Mentha arvensis |
| | var. piperascens |
| Fusarium sp. | Picea glehnii, red clover, Acacia, morning glory, azuki |
| | bean, aster, Thujopsis dolabrata, Asparagus officinalis, flax, |
| | alfalfa, millet, Juncus decipiens, strawberry, Ginkgo biloba, |
| | rice, orris, bush bean, Aralia cordata, Japanese plum, |
| | lacquer tree, Perilla frutescens, garden pea, Swietenia |
| | macrophylla, barley, Salsola komarovii, okra, carnation, |
| | Gerbera, oak, turnip, pumpkin, larch, cauliflower, citrus, |
| | Canarium album, Chrysanthemum morifolium, Brassica |
| | rapa var. narinosa, Phellodendron amurense, cabbage, |
| | cabbage, cucumber, empress tree, Leucaena leucocephala, |
| | guava, Gladiolus, Castanea crenata, Trifolium incarnatum, |
| | Crocus, mulberry, Sagittaria, poppy, Kenaf, Zelkova, |
| | Japanese umbrella pine, pepper, burdock, Cirsium |
| | dipsacolepis, sesame, Brassica chinensis komatsuna, wheat, |
| | coriander, Amorphophalus konjak, Amorphophalus konjak, |
| | Vigna unguiculata, sweet potato, Colocasia esculenta, sugar |
| | cane, Cactus, Chamaecyparis pisifera, Sansevieria, |
| | Cyclamen, potato, Gypsophila paniculata, Chrysanthemum |
| | coronarium, ginger, Pyrethrum, Cucumis melo conomon, |
| | white clover, sweet pea, water melon, Narcissus, |
| | Cryptomeria japonica, stock, Setcreasea purpuea, celery, |
| | horse bean, Japanese radish, Brassica campestris var. |
| | chinensis, soy bean, Douglas fir, tobacco, onion, timothy, |
| | tulip, ginseng, Chinese artichoke, sugarbeet, Dendrobiums, |
| | Capsicum, wax gourd, spruce, maize, Abies sachalinensis, |
| | Pittosporum tobira, tomato, Dritaenopsis, Eustoma |
| | grandiflorum, butcher's-broom, Pyrus pyrifolia, eggplant, |
| | feverfew, Quercus, Momordica charantia, Robonia |
| | pseudoacasia, Allium tuberosum, carrot, Allium sativum, |
| | green onion, Albizia julibrissin, Hibiscus glutinotextilis, |
| | pineapple, Lotus, Stephania japonica, Rhus succedanea, |
| | parsley, banana, Vanilla, papaya, ornamental cabbage, alder, |
| | pitaya, cypress, Cedrus, Phalaenopsis, Phoenix, beech, |
| | Freesia, dishcloth gourd, safflower, Phaseolus coccineus, |
| | hairly vetch, spinach, poplars, Chrysanthemum frutescens, |
| | oriental melon, Phyllostachys, pines, Cryptotaenia |
| | japonica, Artemisia monogyna, Gymnaster savatieri, |
| | meadow foxtail, Ocimium basilicum, melon, firs, peach, |
| | Paraserianthes falcataria, Dioscorea japonica, Lagenaria |
| | siceraria, Eucalyptus, Liliaceae species, Allium bakeri, |
| | apple, Rudbeckia, lupine, Tweedia caerulea, lettuce, redtop, |
| | Hibiscus sabdariffa L., cotton |
| Botrytis sp. | Aucuba japonica, Picea glehnii, red clover, morning glory, |
| | Hydrangea, Angelica keiskei, azuki bean, aster, Astilbe, |
| | Asparagus officinalis, Anemone, flax, arabis, alsike clover, |
| | Alstroemeria, alfalfa, Armeria, isotoma, strawberry, fig, |
| | orris, bush bean, Ficus elastica, Impatiens, Japanese plum, |
| | Perilla frutescens, Evolvulus, garden pea, Prunus avium, |
| | barley, Mirabilis jalapa, Osteospermum, columbine, |
| | carnation, Gerbera, Japanese persimmon, Gazania, |
| | Cattleya, pumpkin, Calla lily, larch, Kalanchoe, quince, |
| | Calceolaria, citrus, Campanula, kiwi fruits, Platycodon, |
| | Chrysanthemum morifolium, ivy, funkia, cabbage, |
| | cucumber, Nerium indicum, Cyrtanthus, Antirrhinum, |
| | calendula, Osmanthus fragrans, Indian cress, Gardenia |
| | jasminoides, hellebores, Trifolium incarnatum, cleome, |
| | clematis, gloxinia, Nigella damascena, Crocus, mulberry, |
| | poppy, Kenaf, cosmos, wheat, common vetch, sweet potato, |
| | Colocasia esculenta, Cactus, Salvia, Cyclamen, Zinnia, |
| | cineraria, potato, Rhododendrons, peony, Trachycarpus |
| | fortunei, white clover, Symphoricarpos, Sweet sultan, sweet |
| | pea, Narcissus, Scabiosa, Cryptomeria japonica, Statice, |
| | Steirodiscus, stock, Spathiphyllum, violet, plum, Sequoia, |
| | Sequoiadendron, Sesbania, mallow, Geranium, Oenanthe |
| | javanica, other clovers, Fagopyrum, horse bean, Cannabis, |
| | Douglas fir, holly hock, tobacco, onion, Dahlia, tea, tulip, |
| | ivy, azaleas, jute, Tetragonia tetragonoides, Basella rubra, |
| | daisy, Delphinium, sugarbeet, Dendrobiums, Capsicum, |
| | spruce, passion flower, Abies sachalinensis, tomato, |
| | Eustoma grandiflorum, Torenia, trefoil, butcher's-broom, |
| | Pyrus pyrifolia, eggplant, rapeseed, Stewartia |
| | pseudocamellia, Madagascar periwinkle, Allium tuberosum, |
| | Prunus japonica, green onion, green onion, Ligustrum |
| | japonicum, Nematanthus, verbena, Cotinus coggygria, |
| | Lotus, parsley, Passiflora edulis, Paphiopedilum, rosids, |
| | Hyacinth, cypress, Ricinus, Cedrus, sunflower, sunflower, |
| | Stemona japonica, Hypericum monogynum, loquat, |
| | Phalaenopsis, Bouvardia, Petasites japonica, Fuchsia, |
| | Chinese Hibiscus, grape, Freesia, Primula species, |
| | blueberry, Didiscus caecaeruleus, Browallia, Begonia, |
| | Petunia, Hemerocallis, hairly vetch, Helichrysum, veronica, |
| | Pentas, Euphorbia pulcherima, Japanese big-leaf magnolia, |
| | Japanese tree peony, hop, toad lily, Ammi majus, yew, hairy |
| | mountainmint, Lupinus mutabilis, pines, marigolds, cornels, |
| | Cryptotaenia japonica, Mimulus, Gymnaster savatieri, |
| | Mesembryanthemum, Ocimium basilicum, Melampodium, |
| | melon, firs, peach, Corchorus olitorius, yacon, Ribes |
| | fasciculatum, Eucalyptus, Eucharis, nankin cherry, |
| | Liliaceae species, ryegrass, lilac, peanut, Allium bakeri, |
| | Ranunculus, Licorice, apple, gentian, lupine, lettuce, |
| | Forsythia, Astragalus, Leucanthemum paludosum, Lobelia, |
| | horse radish, Myosotis, introduced spruce species |
| Aspergillus sp. | fig, rice, persimmon, citrus, Narcissus, onion, tulip, |
| | butcher's-broom, Hyacinth, grape, mango, peach, Liliaceae |
| | species, peanut, apple, cotton |
| Magnaporthe sp. | Setaria italica, rice, Setaria species, Avena sativa, barley, |
| | Shibataea kumasaca, yard grass, Chimonobambusa |
| | marmorea, Pennisetum clandestinum, millet, millet species, |
| | wheat, bamboo grass species, cut-grass species, Eleusine |
| | coracana, ginger, sweet vernal grass, Paspalum thunbergii |
| | Kunth species, Centipede grass, Stenotaphrum secundatum, |
| | timothy, maize, Semiarundinaria fastuosa, Eriochloa |
| | villosa, Coix lacryma-jobi, Panicum, Echinochloa |
| | esculenta, Kyllinga brevifolia, fescue, buffelgrass, |
| | bromegrass, velvet grass, Zizania latifolia, Phyllostachys, |
| | Zingiber mioga, Arundinaria, crabgrass species, ryegrass, |
| | lovegrass, reed canary grass |
| Alternaria sp. | Firmiana platanifolia, azuki bean, aster, Anemone, flax, |
| | Allium species, Alstroemeria, alfalfa, strawberry, rice, orris, |
| | bush bean, Impatiens, Aralia cordata, garden pea, Prunus |
| | avium, Orychophragmus violaceus, okra, carnation, |
| | Gypsophila, turnip, pumpkin, larch, citrus, Catalpa ovata, |
| | cabbage, cucumber, calendula, kudzu, Celosia, poppy, |
| | cosmos, sesame, wheat, sweet potato, salvia, Zinnia, |
| | cineraria, potato, peony, Gypsophila paniculata, |
| | Chrysanthemum coronarium, Senecio cineraria, Gynura |
| | bicolor, Cryptomeria japonica, stock, violet, pear, |
| | Geranium, Gomphrena globosa, other Brassicaceae plants, |
| | horse bean, Japanese radish, soy bean, Cannabis, Polysias |
| | fruticosa, tobacco, onion, ginseng, sugarbeet, Capsicum, |
| | maize, tomato, Eustoma grandiflorum, Pyrus pyrifolia, |
| | eggplant, rapeseed, Phoenix dactylifera, carrot, Allium |
| | sativum, green onion, Hibiscus, Chinese cabbage, |
| | Amaranthus tricolor, Lotus, ornamental cabbage, rosids, |
| | coreopsis, Belamcanda chinensis, cypress, Ricinus, |
| | sunflower, Phoenix, Petasites japonica, Primula species, |
| | broccoli, Petunia, Euphorbia pulcherima, spinach, poplars, |
| | marigolds, Japanese silverleaf (Farfugium japonicum of |
| | Ligularia stenocephala), Ocimium basilicum, melon, peach, |
| | Indian mulberry, peanut, Allium porrum, apple, gentian, |
| | rutabaga, Lunaria, horse radish, Armoracia rusticana, |
| | cotton |
| Cochliobolus sp. | Millet, rice, orchardgrass, barley, yard grass, Eragrostis |
| | ferruginea, wheat, bamboo grass species, sugar cane, |
| | cut-grass species, Zoysia, teosinte, maize, tall oatgrass, |
| | Bermuda grass, fescue, bromegrass, velvet grass, |
| | wheatgrass, Zizania latifolia, Arundinaria, rye, Rhodes |
| | grass |
| Sclerotium sp. | Red clover, Angelica keiskei, ajuga, azuki bean, Asparagus |
| | officinalis, Abutilon, alsike clover, Alstroemeria, alfalfa, |
| | millet, Amorpha fruticosa, strawberry, fig, rice, orris, |
| | Selaginella tamariscina, bush bean, Impatiens, Aralia |
| | cordata, Coptis japonica, orchardgrass, Gaillardia aristata, |
| | Plantago species, Swietenia macrophylla, Atractylodes |
| | japonica, Osteospermum, columbine, Patrinia villosa, |
| | Patrinia scabiosaefolia, Chlorophytum comosum, Oncidium, |
| | carnation, Gerbera, Physostegia virginiana, Gazania, |
| | pumpkin, Tetrapanax payyriferum, Cyperus species, Calla |
| | lily, Caladium, Heterotropa nipponica, citrus, Campanula, |
| | Canarium album, Chrysanthemum morifolium, Helianthus |
| | tuberosus, corydalis, Potentilla fragarioides, Astragalus |
| | membranaceus, funkia, cassava, cucumber, Cyrtanthus, |
| | Antirrhinum, Clerodendrum trichotomum, camphor tree, |
| | Gardenia jasminoides, Gladiolus, hellebores, clematis, |
| | mulberry, mulberry, Clivia, Kenaf, Magnolia kobus, sesame, |
| | Indigofera pseudotinctoria, wheat, colchicum, |
| | Amorphophalus konjak, Vigna unguiculata, Camellia |
| | sasanqua, sweet potato, sugar cane, Sarcococca, |
| | Sandersonia, Digitalis, Eleusine coracana, Dalbergia |
| | sissoo, Zinnia, Zoysia, Phlox subulata, potato, peony, dwarf |
| | lilyturf, Japanese anemone, Pyrethrum, white clover, |
| | Lycopus lucidus, Daphne, water melon, Cryptomeria |
| | japonica, sycamore, Statice, Stokes' aster, violet, Achillea |
| | milefolium, chinaberry, Bidens, senna, Sarcandra glabra, |
| | Jasminum grandiflorum, horse bean, Solidaster luteus, |
| | Magnolia grandiflora, Magnolia grandiflora, soy bean, |
| | Cannabis, Duranta repense, Gamblea innovans, holly hock, |
| | tobacco, Sedum morganianum, onion, Dahlia, Helenium |
| | autumnale, dandelions, tea, Toona sinensis, tulip, China |
| | grass, jute, Solanum mammosum, Camellia, Tulbaghia, |
| | Delphinium, sugarbeet, Dendrobiums, Capsicum, maize, tall |
| | oatgrass, passion flower, Abies sachalinensis, tomato, |
| | Tritoma, Eustoma grandiflorum, trefoil, Pyrus pyrifolia, |
| | eggplant, Momordica charantia, Robonia pseudoacasia, |
| | Madagascar periwinkle, Allium tuberosum, carrot, Allium |
| | sativum, green onion, green onion, nerines, Cirsium |
| | japonicum, Ampelopsis, burclover, Lespedeza, Serissa |
| | foetida, Magnolia heptapeta, banana, papaya, Crinum, |
| | Glehnia littoralis, coreopsis, vanda, alder, Hyacinth, |
| | Belamcanda chinensis, Trapa japonica, cypress, Ricinus, |
| | sunflower, loquat, Phalaenopsis, Petasites japonica, |
| | Eupatorium fortunei, Japanese spurge, grape, bluegrass, |
| | Phaseolus coccineus, Peperomia, Heliopsis, Helichrysum, |
| | Sedum erythrosticum, Penstemon, Bambusa, Physalis, |
| | Japanese tree peony, toad lily, poplars, Urena, Phormium |
| | tenax, Phormium tenax, oriental melon, Zizania latifolia, |
| | pines, Hepatica, Edgeworthia, Gymnaster savatieri, mints, |
| | catchfly, Tradescantia, Farfugium japonicum of Ligularia |
| | stenocephala, meadow foxtail, crabgrass species, melon, |
| | Sorghum bicolor, yacon, Centaurea cyanus, Fatsia, |
| | Liliaceae species, Bauhinia variegata, Allium bakeri, apple, |
| | Hibiscus sabdariffa L. |
| Mycosphaerella sp. | Hydrangea, azuki bean, Thujopsis dolabrata, Aleurites, |
| | flax, Damnacanthus indicus, millet, Meliosma myrianth, |
| | Idesia polycarpa, strawberry, Ficus erecta, orris, bush bean, |
| | Acanthopanax, garden pea, Prunus avium, barley, Shibataea |
| | kumasaca, carnation, maple, Japanese persimmon, pumpkin, |
| | Pourthiaea villosa, larch, quince, citrus, cucumber, Tarenna |
| | gracilipes, empress tree, kudzu, Gardenia jasminoides, |
| | Gladiolus, Castanea crenata, Lindera umbellata, mulberry, |
| | Zelkova, Japanese umbrella pine, wheat, Cleyera japonica, |
| | cherries, pomegranate, Vigna unguiculata, bamboo grass |
| | species, sweet potato, Colocasia esculenta, Kadsura |
| | japonica, Smilax china, Pterocarya rhoifolia, Illicium |
| | religiosum, hornbeams, Tilia japonica, peony, Vaccinium |
| | bracteatum, ginger, Melilotus alba, water melon, |
| | Cryptomeria japonica, currant, sycamore, pear, soy bean, |
| | Polygonaceae, tea, sugarbeet, wax gourd, Fraxinus |
| | japonica, tomato, Pyrus pyrifolia, Ilex chinensis, Quercus, |
| | Rhus javanica, green onion, Lyonia ovalifolia ssp. neziki, |
| | Ligustrum japonicum, Corylus, Cercis, banana, papaya, |
| | rosids, Kalopanax, alder, Ricinus, Japanese spurge, beech, |
| | Abutilon, Chaenomelis fructus, poplars, oriental melon, |
| | Phyllostachys, Actinidia polygama, Cydonia oblonga, |
| | cornels, Staphylea bumalda, Zingiber mioga, melon, firs, |
| | peach, willows, Ardisia japonica, Myrica rubra, Lagenaria |
| | siceraria, Daphniphyllum macropodum, peanut, Allium |
| | bakeri, lapacho, Clethra barbinervis, apple, cotton |
| Cladosporium sp. | Almond, apricot, rice, Japanese plum, Prunus avium, barley, |
| | cacao, Japanese persimmon, pumpkin, larch, citrus, |
| | cucumber, Castanea crenata, wheat, bamboo grass species, |
| | sweet potato, Colocasia esculenta, peony, peony, |
| | Caesalpinia japonica, honeysuckle, plum, Citrus tachibana, |
| | Machilus, Aralia elata, teak, timothy, ginseng, tomato, |
| | butcher's-broom, Pyrus pyrifolia, Passiflora edulis, loquat, |
| | grape, spinach, Japanese tree peony, poplars, oriental melon, |
| | Phyllostachys, melon, peach, Sorghum bicolor, Dioscorea |
| | japonica, Spiraea thunbergii, Daphniphyllum macropodum, |
| | rye, apple, Astragalus |
| Verticillum sp. | Chenopodium species, Thujopsis dolabrata, avocado, |
| | alfalfa, strawberry, Aralia cordata, Prunus avium, okra, |
| | Patrinia scabiosaefolia, Gerbera, maple, cacao, turnip, |
| | Senecio, Platycodon, Chrysanthemum morifolium, cabbage, |
| | cucumber, Antirrhinum, calendula, poppy, cosmos, burdock, |
| | Cirsium dipsacolepis, Japanese hemlock, Perilla, Tilia |
| | japonica, potato, Rhododendrons, Shasta daisy, pyrethrum, |
| | Cucumis melo conomon, sweet pea, water melon, stock, |
| | plum, celery, Japanese radish, soy bean, Aralia elata, |
| | Dahlia, Delphinium ajacis, Agaricus bisporus, Solanum |
| | mammosum, Basella rubra, Delphinium, Capsicum, tomato, |
| | aconite, eggplant, Pholiota nameko, Chinese cabbage, |
| | parsley, ornamental cabbage, rosids, sunflower, Petasites |
| | japonica, grape, grape, broccoli, safflower, Helichrysum, |
| | spinach, Physalis, hop, oriental melon, melon, Corchorus |
| | olitorius, yacon, Liatris, apple, Rudbeckia, Echinops ritro, |
| | lettuce, Armoracia rusticana, cotton |
| Microsphaera sp. | Aucuba japonica, Cocculus trilobus, red clover, Mallotus |
| | japonicus, Akebia quinata, hophornbeam, Hydrangea, |
| | Meliosma myrianth, Ligustrum, Lonicera gracilipes Miq., |
| | Acanthopanax, Deutzia, Styrax japonica, Tilia |
| | maximowicziana, Lonicera vidalii, oak, Viburnum, |
| | Pourthiaea villosa, Viburnum sargenti, Lycium chinense, |
| | camphor tree, Berchemia racemosa, Castanea crenata, |
| | walnut, coyotillo, Lindera umbellata, hovenia, |
| | Menispermum dauricum, Orixa japonica, Phyllanthus |
| | flexuosus, Magnolia kobus, Pterocarya rhoifolia, |
| | Castanopsis, hornbeams, sweet pea, honeysuckle, sycamore, |
| | Vaccinium smallii, horse beans, Stephania cepharantha, |
| | Buckleya lanceolata, abelia, azaleas, Sinomenium, Quercus, |
| | Picrasma quassioides, Robonia pseudoacasia, Sambucus |
| | sieboldiana, burclover, mock-orange, Symplocos prunifolia, |
| | Magnolia heptapeta, Corylus, Helwingia japonica, alder, |
| | Secrinega suffruticosa, Corylopsis pauciflora, Japanese |
| | big-leaf magnolia, Mahonia fortunei, Schisandra nigra, |
| | spindle tree, cornels, Staphylea bumalda, Callicarpa |
| | japonica, Caragana, Berberis, Cinnamomum japonicum, |
| | lilac, lilac, Clethra barbinervis, Astragalus |
| Blumeria sp. | Hystrix longearistata, orchardgrass, barley, wheat grass, |
| | wheat, bromegrass, timothy, bluegrass, wheatgrass, |
| | ryegrass, rye, reed canary grass |
| Erysiphe sp. | red clover, Acacia, Mallotus japonicus, azuki bean, Hystrix |
| | longearistata, flax, alfalfa, bush bean, Deutzia, garden pea, |
| | Coptis japonica, orchardgrass, Plantago species, barley, |
| | Mirabilis jalapa, columbine, oak, turnip, leaf mustard, |
| | Kalanchoe, birch, Chrysanthemum morifolium, Astragalus |
| | membranaceus, cabbage, cucumber, calendula, camphor |
| | tree, Trifolium incarnatum, cleome, clematis, Celosias, |
| | poppy, Indigofera pseudotinctoria, wheat, common vetch, |
| | Vigna unguiculata, Aster tataricus, Phlox subulata, |
| | Filipendula multijuga, peony, white clover, Melilotus alba, |
| | sweet pea, bromegrass, Vaccinium smallii, plum, Achillea |
| | milefolium, other Brassicaceae plants, Fagopyrum, horse |
| | beans, Japanese radish, soy bean, holly hock, Polygonaceae, |
| | Weigela, tobacco, teak, timothy, Datura, China grass, |
| | Oenothera tetraptera, Buckleya lanceolata, Delphinium, |
| | tomato, aconite, trefoil, Abelmoschus manihot, eggplant, |
| | rapeseed, Robonia pseudoacasia, Sambucus sieboldiana, |
| | carrot, beggarweed, Ampelopsis, burclover, Lespedeza, |
| | Chinese cabbage, Weigela coraeensis, Lotus, Stephania |
| | japonica, parsley, ornamental cabbage, alder, sunflower, |
| | bindweeds, grape, beech, bluegrass, Phlox, veronica, Sedum |
| | erythrosticum, wheatgrass, Physalis, Peucedanum |
| | japonicum, Circaea mollis, Farfugium japonicum of |
| | Ligularia stenocephala, Corchorus olitorius, Monarda |
| | fistulosa, Dictyopteris, Cinnamomum japonicum, |
| | Amphicarpaea edgeworthii, Isodon, Aster novi-belgii, |
| | Saxifraga, tulip tree, mugwort, rye, Ranunculus, reed canary |
| | grass, rutabaga, lupine, rosemary, Armoracia rusticana |
| Oidium sp. | Aucuba japonica, Acacia, Mallotus japonicus, Mallotus |
| | japonicus, Solidago virgaurea, Hydrangea, Angelica |
| | keiskei, Ajuga, avocado, flax, Chenopodium ambrosioides, |
| | Albizia saman, wild rosemary, Amorpha fruticosa, Maackia |
| | amurensis, Ficus erecta, Impatiens, Exochorda, Lamium |
| | album, Patrinia scabiosaefolia, carnation, Gazania, |
| | Cercidiphyllum japonicum, Photinia glabra, turnip, |
| | pumpkin, camomile, leaf mustard, Trichosanthes |
| | cucumeroides, Humulus lupulus, citrus, Chrysanthemum |
| | morifolium, Chrysanthemum morifolium, Stachyurus |
| | praecox, cassava, cabbage, cucumber, Antirrhinum, |
| | Hypericum patulum, Lycium chinense, poppy, Murraya |
| | paniculata, Kenaf, Stephanandra incisa, sesame, Indigofera |
| | pseudotinctoria, Citrullus colocynthis, salvia, Zinnia, Phlox |
| | subulata, Rhododendrons, Venidium fastuosum, perennial |
| | aster, Gypsophila paniculata, currant, Scoparia, sycamore, |
| | sycamore, Statice, violet, sage, African violet, other |
| | Brassicaceae plants, Fagopyrum, horse bean, Japanese |
| | radish, soy bean, tobacco, Stephania cepharantha, tamarind, |
| | Dahlia, Helenium autumnale, chervil, Datura, Oenothera |
| | tetraptera, abelia, azaleas, Solanum mammosum, |
| | jewelweeds, dill, dill, sturt's desert pea, Delphinium, |
| | sugarbeet, Coriaria japonica, Aesculus turbinata, tomato, |
| | Eustoma grandiflorum, Torenia, eggplants, Millettia |
| | japonica, Sapium sebiferum, Nierembergia, Momordica |
| | charantia, Albizia julibrissin, yarrow, Ampelopsis, verbena, |
| | Chinese cabbage, Cercis, Eschscholtzia, papaya, Rosa |
| | rugosa, Hevea, rosids, Litsea acuminate, sunflower, |
| | Brachycome iberichfolia, Hypericum monogynum, fennel, |
| | Wisteria, Eupatorium fortunei, grape, Phlox, Begonia, |
| | Petunia, Bixa orellana, Mahonia fortunei, hop, Ammi majus, |
| | Euonymus japonica, Pasania edulis, spindle tree, mango, |
| | cornels, mints, Stauntonia hexaphylla, Leonurus sibiricus, |
| | Melampodium, Hibiscus coccineus, Corchorus olitorius, |
| | Lapsana humilis, Cinnamomum japonicum, Trochodendron |
| | aralioides, Eucalyptus, Daphniphyllum macropodum, |
| | ryegrass, Liatris, Linaria, Lunaria, lemon balm, rosemary, |
| | Rhodanthe, horse radish, Myosotis |
| Sphaerotheca sp. | Thistles, azuki bean, flax, Gynostemma pentaphyllum, |
| | strawberry, bush bean, Japanese plum, Gerbera, oak, |
| | pumpkin, camomile, Helianthus tuberosus, cucumber, |
| | Filipendula purpurea, calendula, Agrimonia pilosa, |
| | Clerodendrum trichotomum, cleome, Stephanandra incisa, |
| | cosmos, burdock, Vigna unguiculata, Aster tataricus, |
| | Zinnia, cineraria, meadow sweet, Cucumis melo conomon, |
| | water melon, currant, Dahlia, China grass, jute, wax gourd, |
| | Torenia, eggplant, Quercus, verbena, papaya, Vigna marina, |
| | Rosa rugosa, rosids, rosids, rosids, coreopsis, sunflower, |
| | Petasites japonica, Prenanthes acerifolia, Hibiscus |
| | mutabilis, Phlox, dishcloth gourd, Petunia, safflower, |
| | Impatiens balsamina, hop, oriental melon, Hibiscus |
| | syriacus, Callicarpa japonica, Farfugium japonicum of |
| | Ligularia stenocephala, melon, peach, Corchorus olitorius, |
| | Lagenaria siceraria, Eucalyptus, Aster novi-belgii, Spiraea |
| | thunbergii, Lactuca indica, Sanguisorba officinalis |
| Phyllactinia sp. | Hophornbeam, Sorbus alnifolia, Schizophragma |
| | hydrangeoides, Alangium platanifolium, Alangium |
| | platanifolium, Celtis sinensis, Pterostyrax hispida, |
| | Pterostyrax hispida, maple, Japanese persimmon, oak, |
| | quince, birch, empress tree, Castanea crenata, walnut, |
| | Lindera umbellata, mulberry, Broussonetia kazinoki, |
| | Magnolia kobus, Amelanchier asiatica, Pterocarya |
| | rhoifolia, Zanthoxylum, hornbeams, hornbeams, |
| | Caesalpinia japonica, Ailanthus altissima, pear, azaleas, |
| | Enkianthus perulatus, Asclepias curassavia, Fraxinus |
| | japonica, Pyrus pyrifolia, Quercus, Sapium sebiferum, |
| | Picrasma quassioides, Lyonia ovalifolia ssp. neziki, |
| | Platycarya strobilacea, Corylus, alder, Corylopsis |
| | pauciflora, Euptelea polyandra, beech, Japanese big-leaf |
| | magnolia, Elliottia paniculata, poplars, Euonymus japonica, |
| | Actinidia polygama, Hamamelis, cornels, peach, Menziesia |
| | purpurea |
| Uncinula sp. | Firmiana platanifolia, Hydrangea, Schizophragma |
| | hydrangeoides, lacquer tree, Celtis sinensis, Pterostyrax |
| | hispida, Tilia maximowicziana, Toisusu urbaniana, maple, |
| | oak, Viburnum, birch, Zelkova, Salix koriyanagi, |
| | Lagerstroemia, hornbeams, Tilia japonica, Toona sinensis, |
| | Buckleya lanceolata, Celastrus orbiculatus, Fraxinus |
| | japonica, Quercus, Picrasma quassioides, Ulmus, Rhus |
| | javanica, Ampelopsis, Styrax obassia, Cotinus coggygria, |
| | Weigela coraeensis, Rhus succedanea, rosids, rosids, alder, |
| | Wisteria, grape, beech, poplars, Actinidia polygama, |
| | Staphylea bumalda, Aphananthe aspera, willows, Celtis |
| | sinensis, maple, mulberry, Alangium platanifolium, |
| | Ailanthus altissima, teak, poplars, Koelreuteria paniculata, |
| | willows |
| Didymella sp. | Citrus, pumpkin, tomato, beech, Myrica rubra, Aesculus |
| | turbinata, Euonymus japonica, bamboo grass species, |
| | bamboo grass species, Phyllostachys, Arundinaria, |
| | cucumber, Cucumis melo conomon, water melon, wax |
| | gourd, Momordica charantia, oriental melon, melon, |
| | Lagenaria siceraria, Quercus |
| Elsinoe sp. | Aralia cordata, Poncirus trifoliata, citrus, guava, walnut, |
| | Broussonetia kazinoki, sweet potato, sugar cane, soy bean, |
| | Aralia elata, tea, rosids, grape, Euonymus japonica, cornels, |
| | Aralia elata |
| Penicillium sp. | Asparagus officinalis, Allium species, rice, orris, |
| | orchardgrass, Rhodea japonica, olive, citrus, Gladiolus, |
| | walnut, Crocus, sweet potato, Narcissus, Citrus tachibana, |
| | tulip, tulip, Basella rubra, maize, tomato, Eustoma |
| | grandiflorum, Allium sativum, pineapple, Hyacinth, grape, |
| | Freesia, spinach, melon, Dioscorea japonica, Liliaceae |
| | species, apple |
| Podosphaera sp. | Sorbus alnifolia, apricot, Japanese plum, Prunus avium, |
| | Cercidiphyllum japonicum, Viburnum, Pourthiaea villosa, |
| | Amelanchier asiatica, cherries, hawthorn, meadow sweet, |
| | Vaccinium smallii, Malus toringo, plum, Sorbus commixta, |
| | Ulmus, Prunus japonica, Malus halliana, Cydonia oblonga, |
| | peach, nankin cherry, apple |
| Phomasp. | Artichoke, Aucuba japonica, red clover, morning glory, |
| | morning glory, Hydrangea, Ajuga, Thujopsis dolabrata, |
| | Asparagus officinalis, flax, Amacrinum, Amaranthus, |
| | alfalfa, Juncus decipiens, rice, Aralia cordata, Japanese |
| | plum, Styrax japonica, garden pea, Pulsatilla cernus, okra, |
| | maple, cacao, Japanese persimmon, oak, larch, citrus, |
| | Campanula, birch, Chrysanthemum morifolium, cassava, |
| | cabbage, empress tree, Castanea crenata, walnut, mulberry, |
| | poppy, Alpinia zerumbet, Rheum, oriental arbor vitae, |
| | burdock, sesame, Brassica chinensis komatsuna, bamboo |
| | grass species, Cyclamen, potato, Narcissus, Achillea |
| | milefolium, Geranium, celery, Cnidium officinale, |
| | Sarcandra glabra, Jasminum grandiflorum, soy bean, |
| | Cannabis, holly hock, Delphinium ajacis, tea, ginseng, |
| | sugarbeet, Capsicum, Angelica acutiloba, Sinobambusa |
| | tootsik, tomato, Pyrus pyrifolia, eggplant, rapeseed, |
| | Nandina domestica, Momordica charantia, carrot, Thuja |
| | standishii, Campsis grandiflora, Lotus, papaya, Crinum, |
| | cypress, Vinca minor, Albizia lebbeck, Garcinia spicata, |
| | Wisteria, Beta vulgaris var. cicla, grape, Abutilon, Bambusa, |
| | spinach, Physalis, Phyllostachys, Farfugium japonicum of |
| | Ligularia stenocephala, Metasequoia, peach, Monstera |
| | deliciosa, Antidesma japonicum, Saxifraga, Phaseolus |
| | limensis, apple, lettuce, Forsythia, horse radish, Mentha |
| | arvensis var. piperascens |
| Phomopsis sp. | Aucuba japonica, Asparagus officinalis, Aleurites, fig, rice, |
| | Rhodea japonica, maple, Japanese persimmon, pumpkin, |
| | larch, citrus, kiwi fruits, cassava, cucumber, empress tree, |
| | Osmanthus fragrans, Gardenia jasminoides, walnut, |
| | mulberry, cosmos, Japanese white pine, cherries, sweet |
| | potato, Ailanthus altissima, water melon, currant, Statice, |
| | pear, soy bean, Douglas fir, tea, Capsicum, Abies |
| | sachalinensis, Pyrus pyrifolia, eggplant, Nandina |
| | domestica, papaya, alder, juniper, Phoenix, grape, poplars, |
| | Cydonia oblonga, mango, cornels, Hibiscus syriacus, |
| | melon, firs, peach, Terminalia catappa, Paraserianthes |
| | falcataria, willows, kerria, Lagenaria siceraria, apple |
| Venturia sp. | Mulberry, sugar cane, pear, Pyrus pyrifolia, Rhus javanica, |
| | poplars, poplars, willows, apple |
| Calonectoria sp. | Acacia, alfalfa, rice, Citysus scoparius, barley, cacao, |
| | Kentia palm, bamboo grass species, soy bean, Glycine, |
| | Robonia pseudoacasia, pines, peanut |
| Rosellinia sp. | Aucuba japonica, Ilex macropoda, Picea glehnii, Mallotus |
| | japonicus, Hydrangea, Asparagus officinalis, avocado, |
| | Halesia carolina, Meliosma myrianth, apricot, Idesia |
| | polycarpa, Taxus cuspidata, fig, Ginkgo biloba, |
| | Cephalotaxus drupacea, Ilex crenata, Podocarpus |
| | macrophyllus, Ligustrum, Impatiens, Lonicera gracilipes |
| | Miq., Deutzia, Japanese plum, lacquer tree, Styrax japonica, |
| | Celtis sinensis, Sophora japonica, Prunus avium, |
| | Pterostyrax hispida, Tilia maximowicziana, Rhodea |
| | japonica, olive, maple, maple, Japanese persimmon, |
| | Dendropanax trifidus, oak, Cercidiphyllum japonicum, |
| | Photinia glabra, Viburnum, Pourthiaea villosa, Torreya |
| | nucifera, larch, quince, citrus, Diplomorpha, kiwi fruits, |
| | Chrysanthemum morifolium, Phellodendron amurense, |
| | Stachyurus praecox, Osmanthus fragrans, camphor tree, |
| | Gardenia jasminoides, Elaeagnus, Castanea crenata, |
| | coyotillo, Lindera umbellata, Lindera umbellata, mulberry, |
| | poppy, Zelkova, coffee, Magnolia kobus, Japanese hemlock, |
| | Japanese hemlock, Amorphophalus konjak, cherries, |
| | bamboo grass species, sweet potato, Chamaecyparis |
| | pisifera, Zanthoxylum, Illicium religiosum, hornbeams, Tilia |
| | japonica, meadow sweet, potato, peony, Raphiolepis |
| | umbellata, Sapium japonicum, white clover, Neolitsea |
| | sericea, Rhodotypos scandens, Ailanthus altissima, Daphne, |
| | Narcissus, Cryptomeria japonica, Pinus strobus, Malus |
| | toringo, plum, pear, Ilex aquifolium, Sarcandra glabra, |
| | Jasminum grandiflorum, horse bean, soy bean, Cannabis, |
| | tobacco, tea, ginseng, China grass, azaleas, spruce, |
| | Loropetalum chinense, Aesculus turbinata, Abies |
| | sachalinensis, Abies sachalinensis, Fraxinus japonica, |
| | Pittosporum tobira, Podocarpus nagi, Pyrus pyrifolia, Ilex |
| | chinensis, Quercus, Euonymus alatus, Ulmus, Sambucus |
| | sieboldiana, Ligustrum japonicum, Albizia julibrissin, |
| | mock-orange, Styrax obassia, Weigela coraeensis, Corylus, |
| | Rhus succedanea, Malus halliana, Eurya emarginata, |
| | rosids, Kalopanax, alder, Osmanthus ilicifolius, |
| | Chionanthus retusus, cypress, Stewartia monadelpha, |
| | Corylopsis pauciflora, Pyracantha, loquat, grape, beech, |
| | Japanese big-leaf magnolia, Chaenomelis fructus, Machilus |
| | japonica, Mahonia fortunei, poplars, Euonymus japonica, |
| | Phyllostachys, pines, mate, Pasania edulis, spindle tree, |
| | Hamamelis, cornels, Edgeworthia, Aphananthe aspera, |
| | Callicarpa japonica, firs, peach, Fatsia, willows, |
| | Trochodendron aralioides, Daphniphyllum macropodum, |
| | tulip tree, peanut, Clethra barbinervis, apple, lupine, |
| | Forsythia, introduced spruce species |
| Claviceps sp. | Rice, Eccoilopus, Hakonechloa macra, orchardgrass, |
| | Spodiopogon, barley, Trisetum species, Phalaris species, |
| | wheat, bamboo grass species, Zoysia, Miscanthus species, |
| | Alopecurus species, Paspalum thunbergii, Kunth species, |
| | Isachne species, Oplismenus species, timothy, tall oatgrass, |
| | Arundinella hirta species, Agrostis species, Calamagrostis |
| | species, paspalum, Elymus species, Polypogon species, |
| | Capillipedium species, fescue, Scirpus tabernaemontani, |
| | bluegrass, bromegrass, velvet grass, meadow foxtail, |
| | crabgrass species, Sorghum bicolor, reeds, ryegrass, rye, |
| | reed canary grass, redtop |
| Ustilago sp. | Reed canary grass, millet (Echinochloa sp., Eleusine sp.), |
| | redtop, Aegilops sp., Avena sp., Cinnamomum japonicum, |
| | Sorghum bicolor, fig, orchardgrass, timothy, bluegrass, |
| | barley, Setaria italica, bamboo grass species, sugar cane, |
| | jobster (Coix lacryma-jobi), Stenotaphrum secundatum, |
| | teosinte, maize, Bermuda grass, millet, bromegrass, |
| | Machilus japonica, Zizania latifolia, Phyllostachys, |
| | Arundinaria, barley, wheat, tall oatgrass, Sorghum bicolor |
| Helicobasidium sp. | Aucuba japonica, Firmiana platanifolia, red clover, |
| | Mallotus japonicus, Asparagus officinalis, alfalfa, Meliosma |
| | myrianth, apricot, Amorpha fruticosa, Taxus cuspidata, fig, |
| | Ginkgo biloba, Ficus erecta, Ligustrum, Ficus elastica, |
| | Japanese plum, lacquer tree, Celtis sinensis, Prunus avium, |
| | maple, Japanese persimmon, oak, Photinia glabra, Torreya |
| | nucifera, larch, citrus, Viburnum sargenti, Phellodendron |
| | amurense, cucumber, Nerium indicum, empress tree, |
| | camphor tree, Castanea crenata, walnut, mulberry, Zelkova, |
| | burdock, Japanese white pine, Amorphophalus konjak, |
| | cherries, sweet potato, Chamaecyparis pisifera, |
| | Castanopsis, potato, Raphiolepis umbellata, Sapium |
| | japonicum, white clover, Ailanthus altissima, Melilotus |
| | alba, honeysuckle, Cryptomeria japonica, sycamore, Pinus |
| | strobus, plum, soy bean, tobacco, Aralia elata, tea, China |
| | grass, abelia, Calophyllum inophyllum, sugarbeet, spruce, |
| | Fraxinus japonica, Pittosporum tobira, Pyrus pyrifolia, |
| | Quercus, Thuja, Robonia pseudoacasia, Ulmus, Sambucus |
| | sieboldiana, carrot, Ligustrum japonicum, Platycarya |
| | strobilacea, Lespedeza, Styrax obassia, Rhus succedanea, |
| | Cercis, Hevea, alder, Eurya japonica, cypress, Corylopsis |
| | pauciflora, loquat, Euptelea polyandra, grape, Chaenomelis |
| | fructus, poplars, Asimina triloba, Euonymus japonica, pines, |
| | spindle tree, cornels, Edgeworthia, Aphananthe aspera, |
| | Sapindus mukurossi, Arundinaria, peach, willows, tulip |
| | tree, Liliaceae species, peanut, Pistacia chinensis, apple, |
| | rutabaga, Forsythia, introduced spruce species |
| Phytophthora sp. | Aucuba japonica, Cinchona succirubra, Agapanthus, |
| | Angelica keiskei, azuki bean, Asparagus officinalis, |
| | Cypripedium macranthos, Anemone, Aphelandra, Abutilon, |
| | avocado, Amaryllis, Allium species, Alstroemeria, alfalfa, |
| | Albuka, aloe, apricot, Taxus cuspidata, strawberry, fig, rice, |
| | orris, Impatiens, Aralia cordata, Japanese plum, Echium, |
| | Citysus scoparius, garden pea, Coptis japonica, |
| | Ornithogalum, okra, okra, Hypericum erectum, Oncidium, |
| | carnation, Gerbera, maple, cacao, Japanese persimmon, |
| | Gazania, Gypsophila, Cattleya, Photinia glabra, turnip, |
| | pumpkin, Calla lily, Ardisia crispa, larch, Kalanchoe, citrus, |
| | Campanula, birch, Canarium album, kiwi fruits, |
| | Chrysanthemum morifolium, ivy, Cinchona ledgeriana, |
| | cucumber, Allium victorialis subsp. platyphyllum, |
| | Antirrhinum, calendula, guava, kudzu, Castanea crenata, |
| | gloxinia, Celosia, Murraya paniculata, Kentia palm, Cocos |
| | nucifera, Capsicum annuum cv., pepper, sesame, Crowea, |
| | Colocasia esculenta, Arenga pinnata, Arenga pinnata, |
| | Cactus, Salvia, Zanthoxylum, Sandersonia, Rehmannia |
| | Root, Perilla, Cyperus monophyllus Vahl, potato, |
| | Rhododendrons, peony, Gypsophila paniculata, ginger, |
| | Cucumis melo conomon, Cucumis melo conomon, Daphne, |
| | water melon, Cryptomeria japonica, Statice, stock, |
| | Strelitzia, Spathiphyllum, violet, pear, Melia azedarach, |
| | African violet, Cordyline terminalis, Sarcandra glabra, |
| | Fagopyrum, horse bean, soy bean, Cannabis, tobacco, |
| | onion, Aralia elata, tulip, ginseng, China grass, Solanum |
| | mammosum, Camellia, Basella rubra, Dinema, sugarbeet, |
| | Capsicum, wax gourd, spruce, Fraxinus japonica, tomato, |
| | dracaena, Durio zibethinus, Triteleia, Eustoma |
| | grandiflorum, trefoil, Abelmoschus manihot, Pyrus |
| | pyrifolia, eggplant, Canavalia gladiata, Robonia |
| | pseudoacasia, Madagascar periwinkle, Allium tuberosum, |
| | carrot, Allium sativum, green onion, nerines, pineapple, |
| | parsley, Passiflora edulis, papaya, ornamental cabbage, |
| | Hevea, rosids, vanda, Hyacinth, cypress, Ricinus, sunflower, |
| | Pyracantha, loquat, Philodendron, Bougainvillea, Phoenix, |
| | beech, Penstemon, Euphorbia pulcherima, Abutilon, |
| | spinach, Japanese tree peony, boronia, Ammi majus, oriental |
| | melon, pines, pines, mango, Gymnaster savatieri, |
| | Lithospermum erythrorhizon, Rhoeo discolor, melon, firs, |
| | peach, Myrica rubra, Lagenaria siceraria, Liliaceae |
| | species, Allium bakeri, lavender, Dracaena draco, apple, |
| | Tweedia caerulea, lettuce, Hibiscus sabdariffa L., Allium |
| | fistulosum caespitosum, cotton |
| Pythium sp. | Carrot, strawberry, strawberry, Zoysia, maize, cabbage, |
| | tulip, Chrysanthemum morifolium, pineapple, wheat, |
| | strawberry, alsike clover, Rhoeo discolor, Geranium, |
| | Phaseolus coccineus, sweet potato, Olluco, Alstroemeria, |
| | melon, Japanese plum, Phoenix, Colocasia esculenta, |
| | cornels, flax, Cryptomeria (Japanase cedar), Sagittaria, |
| | redtop, sweet potato, Cattleya, tea, rice, cotton, |
| | Sansevieria, tobacco, bush bean, Fagopyrum, soy bean, |
| | carrot, Gerbera, rosids, ryegrass, redtop, azuki bean, other |
| | Brassicaceae plants, Brassica campestris var. chinensis, |
| | Chinese cabbage, broccoli, Mesembryanthemum, barley, red |
| | clover, alfalfa, Avena sativa, orchardgrass, barley, wheat, |
| | common vetch, white clover, horse bean, timothy, fescue, |
| | bluegrass, hairly vetch, ryegrass, rye, redtop, Astragalus, |
| | Cactus, Geranium, Pachira, Ullucus, maize, lupine, ginger, |
| | Zingiber mioga, Angelica keiskei, Luculia pinceana, Erica |
| | species, orchardgrass, carnation, Kalanchoe, kiwi fruits, |
| | cucumber, Antirrhinum, Asparagaceae, burdock, coleus, |
| | Amorphophalus konjak, Colocasia esculenta, sugar cane, |
| | Sandersonia, horse bean, tulip, dill, maize, tomato, |
| | dracaena, Eustoma grandiflorum, green onion, parsley, |
| | rosids, Bouvardia, Schefflera octophylla, Euphorbia |
| | pulcherima, Euphorbia pulcherima, Valerianella locusta, |
| | Cryptotaenia japonica, yacon, gentian, Canarium album, |
| | Dendrobiums, rapeseed, Gypsophila paniculata, stock, |
| | Picea glehnii, rice, bush bean, lacquer tree, garden pea, |
| | okra, oak, kalmia, birch, cabbage, empress tree, Zelkova, |
| | Japanese white pine, Rhododendrons, onion, Dahlia, |
| | Delphinium, sugarbeet, tomato, Tritoma, Quercus, Rhus |
| | succedanea, alder, dishcloth gourd, poplars, melon, |
| | Eucalyptus, Lagenaria siceraria, Japanese radish, Basella |
| | rubra, Lotus, Peperomia, Bergeranthus, pumpkin, |
| | cucumber, water melon, tomato, Phaseolus coccineus, |
| | Oenanthe javanica, rice, other Brassicaceae plants, papaya, |
| | alfalfa, aster, Brassica rapa var. nipposinica, curcuma, |
| | Zinnia, Chrysanthemum coronarium, white clover, sweet |
| | pea, water melon, violet, Japanese radish, Abelmoschus |
| | manihot, Nemesia, juniper, Petasites japonica, Fuchsia, |
| | Petunia, spinach, lettuce, Astragalus, bush bean, Oenanthe |
| | javanica, Chinese cabbage, Hibiscus sabdariffa L., peanut, |
| | maize, Sorghum bicolor, cocoyam, pepper, cucumber, |
| | Impatiens, Dioscorea japonica, redtop |
| Peronospora sp. | Red clover, alfalfa, garden pea, Salsola komarovii, Brassica |
| | oleracea, turnip, leaf mustard, cauliflower, Chrysanthemum |
| | morifolium, Brassica rapa var. narinosa, cabbage, |
| | hellebores, cleome, poppy, kohlrabi, Kochia, common |
| | vetch, coleus, Cyperus monophyllus Vahl, Chrysanthemum |
| | coronarium, pyrethrum, white clover, Scabiosa, violet, other |
| | Brassicaceae plants, Fagopyrum, horse bean, Japanese |
| | radish, soy bean, Cannabis, tobacco, onion, Delphinium |
| | ajacis, sugarbeet, maize, trefoil, rapeseed, green onion, |
| | burclover, Chinese cabbage, ornamental cabbage, rosids, |
| | Beta vulgaris var. cicla, broccoli, hairly vetch, Helichrysum, |
| | spinach, Mesembryanthemum, rutabaga, Eruca vesicaria, |
| | Astragalus, Allium fistulosum caespitosum, horse radish |
| Pucciniasp. | almond, Mallotus japonicus, Akebia quinata, Hydrangea, |
| | Asparagus officinalis, Eccoilopus, Polygonatum odoratum, |
| | Allium species, apricot, Distylium racemosum, poa, orris, |
| | Lonicera gracilipes Miq., Deutzia, Japanese plum, |
| | Alangium platanifolium, Styrax japonica, Avena sativa, |
| | Prunus avium, Spodiopogon, orchardgrass, orchardgrass, |
| | Pterostyrax hispida, Plantago species, Tilia |
| | maximowicziana, barley, Youngia species, Ohia, Patrinia |
| | scabiosaefolia, carnation, maple, Beckmannia species, |
| | Oxalis, Trisetum species, Viburnum, wheat grass, |
| | Heterosmilax japonica, Thalictrum species, Kalanchoe, |
| | Chimonobambusa marmorea, Canna, bramble, |
| | Chrysanthemum morifolium, Chrysanthemum species, |
| | Justicia species, Stachyurus praecox, funkia, Tarenna |
| | gracilipes, Antirrhinum, Agrimonia pilosa, guava, |
| | Clerodendrum trichotomum, Phalaris species, Berchemia |
| | racemosa, Elaeagnus, Castanea crenata, Coyotillo, Pertya |
| | scandens, Arthraxon species, wheat, Melica species, |
| | Japanese hemlock, Hoya carnosa, cherries, bamboo grass |
| | species, sugar cane, Smilax china, Schoenoplectus triqueter, |
| | Tilia japonica, Zoysia, Chrysanthemum coronarium, |
| | pyrethrum, white clover, Neolitsea sericea, Sweet vernal |
| | grass, currant, Carex species, Miscanthus species, |
| | bromegrass, Paspalum thunbergii Kunth species, violet, |
| | plum, Achillea milefolium, Oenanthe javanica, Fagopyrum, |
| | holly hock, Polygonaceae, onion, Imperata species, |
| | Oplismenus species, timothy, tea, Buxus microphylla var. |
| | japonica, jewelweeds, daisy, Convallaria majalis, |
| | Sinobambusa tootsik, spruce, maize, tall oatgrass, Coriaria |
| | japonica, Abies sachalinensis, feverfew, Stewartia |
| | pseudocamellia, Semiarundinaria fastuosa, Polygonatum |
| | falcatum, Ixeris species, Allium tuberosum, Anemone |
| | flaccida, Vitex cannabifolia, Allium sativum, Agrostis |
| | species, Moliniopsis species, green onion, Sporobolus |
| | species, Calamagrostis species, Sonchus species, Malva |
| | rotundifolia, mock-orange, Styrax obassia, Stellaria species, |
| | Corylus, Passiflora edulis, Epipremnum pinnatum, Premna |
| | japonica, Elymus species, Glehnia littoralis, Bermuda grass, |
| | Eleocharis species, Litsea acuminata, Belamcanda |
| | chinensis, sunflower, Capillipedium species, Stewartia |
| | monadelpha, Corylopsis pauciflora, fescue, Petasites |
| | japonica, Prenanthes acerifolia, Ambrosia species, Scirpus |
| | tabernaemontani, rose apples, beech, Primula species, |
| | bluegrass, bluegrass, bromegrass, Begonia, safflower, |
| | Hemerocallis, velvet grass, Sedum erythrosticum, |
| | wheatgrass, wheatgrass, Impatiens balsamina, Bambusa, |
| | spinach, toad lily, Chrysanthemum frutescens, yew, |
| | Phyllostachys, Actinidia polygama, Hamamelis, cornels, |
| | Lysichiton species, Cryptotaenia japonica, mints, |
| | Stauntonia hexaphylla, Berberis, Farfugium japonicum of |
| | Ligularia stenocephala, Arundinaria, crabgrass species, |
| | firs, peach, Sorghum bicolor, Cinnamomum japonicum, |
| | Liriope muscari, reeds, mugwort, ryegrass, rye, rye, peanut, |
| | Allium bakeri, reed canary grass, Licorice, Lactuca indica, |
| | Clethra barbinervis, gentian, redtop, Adoxa moschtellina, |
| | Syzygium aqueum, Mentha arvensis var. piperascens |
| Uromyces sp. | red clover, azuki bean, Amana species, alsike clover, |
| | alfalfa, Armeria, aloe, millet, Juncus species, Maackia |
| | amurensis, bush bean, lacquer tree, Setaria species, Sophora |
| | japonica, garden pea, orchardgrass, carnation, Erythronium |
| | species, Astragalus membranaceus, millet species, Cajanus, |
| | cassava, Trifolium incarnatum, coyotillo, Fritillaria |
| | camtschatcensis, Indigofera pseudotinctoria, common |
| | vetch, Vigna unguiculata, subterranean clover, cut-grass |
| | species, Dalbergia sissoo, Acorus calamus, white clover, |
| | Melilotus alba, Alopecurus species, Statice, Acacia confusa, |
| | horse bean, horse beans, soy bean, Ehretia ovalifolia, |
| | Commelina species, sugarbeet, aconite, trefoil, Eriochloa |
| | villosa, Rhus javanica, Allium species, burclover, |
| | Lespedeza, Rhus succedanea, Cladrastis Platycarpa, Lablab |
| | purpureus, hairly vetch, Zizania latifolia, Callicarpa |
| | japonica, Caragana, Heliotropium foertherianum, |
| | Dictyopteris, Liliaceae species, Astragalus |

α-1,3-glucanase used in the present invention include wild-type α-1,3-glucanase originating from an organism, a variant thereof, and an active fragment thereof.

“Wild-type α-1,3-glucanase” may originate from any organism species, provided that such known α-1,3-glucanase has activity of hydrolyzing α-1,3-glucan. The amino acid sequence of such known wild-type α-1,3-glucanase or the nucleotide sequence of the wild-type α-1,3-glucanase gene can be obtained by searching GenBank or other databases. Examples thereof include the proteins registered as α-1,3-glucanase of organisms indicated by the GenBank Accession Numbers shown in Table 3 and the genes encoding proteins that are deduced to be α-1,3-glucanase having the amino acid coverage of greater than 80% and the e-value of greater than 100 in relation to α-1,3-glucanase of Tricoderma reesi as a result of BlastX. The reason why the proteins having the amino acid coverage of greater than 80% and the e-value of greater than 100 are designated as α-1,3-glucanase is as follows. That is, the amino acid coverage is greater than 80% and the e-value is greater than 100 among almost all α-1,3-glucanases of various organism species that have been already identified. Alternatively, the nucleotide sequences of the α-1,3-glucanase gene of the Aspergillus fungus disclosed on the Broad Institute (www.broadinstitute.org) indicated by the accession numbers shown in Table 4 can also be used. Specific examples include the α-1,3-glucanase gene comprising the nucleotide sequence as shown in SEQ ID NO: 23 originating from Bacillus circulans KA304 (Paenibacillus sp.) and the α-1,3-glucanase gene originating from α-1,3-glucanase having the amino acid sequence as shown in SEQ ID NO: 31 or the rice blast fungus (Magnaporthe grisea) registered under the GenBank Accession Number XP001410317 or the Broad Institute MGG 12678 (http://www.broadinstitute.org/annotation/genome/magnaporthe_{—}grisea/MultiHome.html; http://www.broadinstitute.org/annotation/genome/magnaporthe_{—}grisea/GeneDetails.html?sp=S7000002168138321).

TABLE 3

| Organism | GenBank (Protein ID) |
|---|---|
| Ajellomyces capsulatus G186AR | EEH09577.1 |
| Ajellomyces dermatitidis ER-3 | EEQ89186.1 |
| Aspergillus clavatus NRRL 1 | XP_001272785.1 |
| Aspergillus flavus NRRL3357 | XP_002372708.1 |
| Aspergillus flavus NRRL3357 | XP_002373672.1 |
| Aspergillus flavus NRRL3357 | XP_002375002.1 |
| Aspergillus flavus NRRL3357 | XP_002376817.1 |
| Aspergillus flavus NRRL3357 | XP_002380999.1 |
| Aspergillus flavus NRRL3357 | XP_002381644.1 |
| Aspergillus fumigatus A1163 | EDP47900.1 |
| Aspergillus fumigatus A1163 | EDP48586.1 |
| Aspergillus fumigatus A1163 | EDP48708.1 |
| Aspergillus fumigatus A1163 | EDP49885.1 |
| Aspergillus fumigatus A1163 | EDP51240.1 |
| Aspergillus fumigatus A1163 | EDP54055.1 |
| Aspergillus fumigatus Af293 | EAL85367.1 |
| Aspergillus fumigatus Af293 | EAL85399.1 |
| Aspergillus fumigatus Af293 | EAL85912.1 |
| Aspergillus fumigatus Af293 | EAL86726.1 |
| Aspergillus fumigatus Af293 | EAL86742.1 |
| Aspergillus fumigatus Af293 | EAL87492.1 |
| Aspergillus fumigatus Af293 | EAL87779.1 |
| Aspergillus fumigatus Af293 | EBA27333.1 |
| Aspergillus fumigatus Af293 | XP_001481700.1 |
| Aspergillus fumigatus Af293 | XP_747405.1 |
| Aspergillus fumigatus Af293 | XP_747437.1 |
| Aspergillus fumigatus Af293 | XP_747950.1 |
| Aspergillus fumigatus Af293 | XP_748780.1 |
| Aspergillus fumigatus Af293 | XP_749530.1 |
| Aspergillus nidulans FGSC A4 | CBF74212.1 |
| Aspergillus nidulans FGSC A4 | CBF84404.1 |
| Aspergillus nidulans FGSC A4 | XP_661394.1 |
| Aspergillus nidulans FGSC A4 | XP_680618.1 |
| Aspergillus nidulans FGSC A4 | XP_682311.1 |
| Aspergillus niger | CAK42453.1 |
| Aspergillus niger | CAK96739.1 |
| Aspergillus niger | XP_001390878.1 |
| Aspergillus niger | XP_001391939.1 |
| Aspergillus niger | XP_001393071.1 |
| Aspergillus niger | XP_001395266.1 |
| Aspergillus niger | XP_001397010.1 |
| Aspergillus oryzae RIB40 | XP_001819520.1 |
| Aspergillus oryzae RIB40 | XP_001824372.1 |
| Aspergillus oryzae RIB40 | XP_001818440.1 |
| Aspergillus oryzae RIB40 | XP_001825675.1 |
| Aspergillus terreus NIH2624 | EAU33738.1 |
| Aspergillus terreus NIH2624 | EAU35458.1 |
| Aspergillus terreus NIH2624 | XP_001212834.1 |
| Aspergillus terreus NIH2624 | XP_001215155.1 |
| Botryotinia fuckeliana B05.10 | XP_001554310.1 |
| Botryotinia fuckeliana B05.10 | XP_001555864.1 |
| Cryptococcus neoformans var. | AAW47079.1 |
| neoformans JEC21 | |
| Cryptococcus neoformans var. | XP_568596.1 |
| neoformans JEC21 | |
| Emericella nidulans | ABF50884.1 |
| Emericella nidulans | CAC48025.1 |
| Laccaria bicolor S238N-H82 | XP_001879683.1 |
| Magnaporthegrisea 70-15 | XP_001410317.1 |
| Moniliophthora perniciosa FA553 | EEB95719.1 |
| Moniliophthora perniciosa FA553 | XP_002394789.1 |
| Moniliophthora perniciosa FA553 | XP_002397873.1 |
| Nectria haematococca mpVI 77-13-4 | EEU34789.1 |
| Neosartorya fischeri NRRL 181 | XP_001260032.1 |
| Neosartorya fischeri NRRL 181 | XP_001262564.1 |
| Neurospora crassa | CAB92025.1 |
| Neurospora crassa OR74A | XP_958818.1 |
| Paracoccidioides brasiliensis | ABS29020.1 |
| Paracoccidioides brasiliensis | ABV57468.1 |
| Paracoccidioides brasiliensis Pb01 | EEH33153.1 |
| Penicillium chrysogenum | CAP83026.1 |
| Wisconsin 54-1255 | |
| Penicillium chrysogenum | CAP91182.1 |
| Wisconsin 54-1255 | |
| Penicillium chrysogenum | CAP92350.1 |
| Wisconsin 54-1255 | |
| Penicillium chrysogenum | CAP94862.1 |
| Wisconsin 54-1255 | |
| Penicillium chrysogenum | XP_002557585.1 |
| Wisconsin 54-1255 | |
| Penicillium chrysogenum | XP_002558141.1 |
| Wisconsin 54-1255 | |
| Penicillium chrysogenum | XP_002558559.1 |
| Wisconsin 54-1255 | |
| Penicillium chrysogenum | XP_002560364.1 |
| Wisconsin 54-1255 | |
| Penicillium chrysogenum | XP_002562465.1 |
| Wisconsin 54-1255 | |
| Penicillium funiculosum | CAD48301.1 |
| Penicillium marneffei ATCC 18224 | XP_002147859.1 |
| Penicillium marneffei ATCC 18224 | XP_002151869.1 |
| Penicillium marneffei ATCC 18224 | XP_002152547.1 |
| Penicillium purpurogenum | AAF27912.1 |
| Penicillium purpurogenum | ABY56286.1 |
| Penicillium purpurogenum | ABY56286.1 |
| Schizosaccharomyces japonicus | XP_002174591.1 |
| Schizosaccharomyces japonicus | XP_002173914.1\| |
| Schizosaccharomyces pombe | AAT84065.1 |
| Schizosaccharomyces pombe | AAT84064.1 |
| | (NP_001018296.1) |
| Sclerotinia sclerotiorum 1980 | XP_001586736.1 |
| Sclerotinia sclerotiorum 1980 | XP_001590360.1 |
| Sclerotinia sclerotiorum 1980 | XP_001597300.1 |
| Sordaria macrospora | CBI53313.1 |
| Talaromyces stipitatus ATCC 10500 | XP_002479103.1 |
| Talaromyces stipitatus ATCC 10500 | XP_002480851.1 |
| Talaromyces stipitatus ATCC 10500 | XP_002482062.1 |
| Trichoderma asperellum | ABG56439.1 |
| Trichoderma asperellum | CAH04880.1 |
| Trichoderma harzianum (Hypocrea lixii) | CAC80493.1 |
| Verticillium albo-atrum VaMs.102 | EEY18429.1 |
| Bacillus circulans | BAE98302.1 |
| Catenulispora acidiphila DSM 44928 | YP_003114142.1 |
| Clostridium phytofermentans ISDg | YP_001558424.1 |
| Geobacillus sp. Y412MC10 | YP_003242479.1 |
| Kribbella flavida DSM 17836 | YP_003378403.1 |
| Kribbella flavida DSM 17836 | YP_003383609.1 |
| Micromonospora aurantiaca ATCC 27029 | ZP_06220379.1 |
| Micromonospora sp. L5 | ZP_06397042.1 |
| Paenibacillus humicus | BAI23187.1 |
| Paenibacillus sp. JDR-2 | YP_003009383.1 |
| Paenibacillus sp. JDR-2 | YP_003012334.1 |
| Paenibacillus sp. KSM-M126 | BAG15879.1 |
| Paenibacillus sp. KSM-M126 | BAG15880.1 |
| Paenibacillus sp. KSM-M318 | BAH10514.1 |
| Paenibacillus sp. KSM-M318 | BAH10514.1 |
| Paenibacillus sp. KSM-M35 | BAG15878.1 |
| Paenibacillus sp. KSM-M86 | BAF56208.1 |
| Paenibacillus sp. oral taxon 786 str. D14 | ZP_04851296.1 |
| Prevotella melaninogenica ATCC 25845 | ZP_04832647.1 |
| Prevotella melaninogenica D18 | ZP_06407273.1 |
| Secreted protein [Streptomyces sp. AA4] | ZP_05479781.1 |
| Stigmatella aurantiaca DW4/3-1 | ZP_01459417.1 |
| Streptococcus salivarius SK126 | ZP_04062804.1 |
| | (EEK09145.1) |
| Streptomyces albus J1074 | ZP_04702042.1 |
| Streptomyces ambofaciens ATCC 23877 | CAJ88733.1 |
| Streptomyces ambofaciens ATCC 23877 | CAJ89945.1 |
| Streptomyces coelicolor A3 (2) | NP_631079.1 |
| Streptomyces coelicolor A3 (2) | NP_631099.1 |
| Streptomyces flavogriseus ATCC 33331 | ZP_05800843.1 |
| Streptomyces ghanaemis ATCC 14672 | ZP_04683887.1 |
| Streptomyces griseus subsp. | YP_001821986.1 |
| griseus NBRC 13350 | |
| Streptomyces griseus subsp. | YP_001828134.1 |
| griseus NBRC 13350 | |
| Streptomyces lividans TK24 | ZP_05522090.1 |
| Streptomyces lividans TK24 | ZP_05522113.1 |
| Streptomyces lividans TK24 | ZP_06526943.1 |
| Streptomyces pristinaespiralis ATCC 25486 | ZP_05010684.1 |
| Streptomyces pristinaespiralis ATCC 25486 | ZP_05013791.1 |
| Streptomyces roseosporus NRRL 11379 | ZP_04697982.1 |
| Streptomyces sp. AA4 | ZP_05479780.1 |
| Streptomyces sp. ACT-1 | ZP_06276139.1 |
| Streptomyces sp. ACT-1 | ZP_06276868.1 |
| Streptomyces sp. ACTE | ZP_06273223.1 |
| Streptomyces sp. SPB74 | ZP_04992779.1 |
| Streptomyces sp. SPB74 | ZP_04993292.1 |
| Streptomyces sp. SPB78 | ZP_05486713.1 |
| Streptomyces sp. SPB78 | ZP_05487086.1 |
| Streptosporangium roseum DSM 43021 | YP_003336569.1 |
| Streptosporangium roseum DSM 43021] | YP_003338829.1 |

TABLE 4

| Organism | Broad Institute (Gene) |
|---|---|
| Aspergillus clavatus | ACLA_090520 |
| Aspergillus clavatus | ACLA_001200 |
| Aspergillus clavatus | ACLA_059460 |
| Aspergillus fumigatus | Afu7g08350 |
| Aspergillus fumigatus | Afu8g06360 |
| Aspergillus fumigatus | Afu5g03940 |
| Aspergillus fumigatus | Afu1g00650 |
| Aspergillus fumigatus | Afu1g03350 |
| Aspergillus fumigatus | Afu7g08510 |
| Aspergillus fumigatus | Afu8g06030 |
| Aspergillus fumigatus | Afu2g03980 |
| Aspergillus fumigatus | Afu2g06160 |
| Aspergillus flavus | AFL2G_09974.2 |
| Aspergillus flavus | AFL2G_04371.2 |
| Aspergillus flavus | AFL2G_08576.2 |
| Aspergillus flavus | AFL2G_08211.2 |
| Aspergillus flavus | AFL2G_08670.2 |
| Aspergillus flavus | AFL2G_01424.2 |
| Aspergillus flavus | AFL2G_00539.2 |
| Aspergillus flavus | AFL2G_08808.2 |
| Aspergillus flavus | AFL2G_02656.2 |
| Aspergillus nidulans | ANID_03790.1 |
| Aspergillus nidulans | ANID_11064.1 |
| Aspergillus nidulans | ANID_09042.1 |
| Aspergillus nidulans | ANID_01604.1 |
| Aspergillus nidulans | ANID_07349.1 |
| Aspergillus niger | gw1_3.133 |
| Aspergillus niger | e_gw1_6.273 |
| Aspergillus niger | gw1_12.42 |
| Aspergillus niger | e_gw1_3.553 |
| Aspergillus niger | est_GWPlus_C_50362 |
| Aspergillus niger | est_GWPlus_C_110147 |
| Aspergillus terreus | ATEG_05977.1 |
| Aspergillus terreus | ATEG_03656. |
| Aspergillus oryzae | AO090023000532 |
| Aspergillus oryzae | AO090113000091 |
| Aspergillus oryzae | AO090120000192 |
| Aspergillus oryzae | AO090113000198 |
| Aspergillus oryzae | AO090005001526 |
| Aspergillus oryzae | AO090003000323 |
| Aspergillus oryzae | AO090005000538 |
| Aspergillus oryzae | AO090102000599 |
| Aspergillus oryzae | AO090138000172 |
| Neosartorya fischeri | NFIA_080790 |
| Neosartorya fischeri | NFIA_031000 |
| Neosartorya fischeri | NFIA_001600 |
| Neosartorya fischeri | NFIA_001590 |
| Neosartorya fischeri | NFIA_005690 |
| Neosartorya fischeri | NFIA_030580 |
| Neosartorya fischeri | NFIA_038180 |
| Neosartorya fischeri | NFIA_001580 |
| Neosartorya fischeri | NFIA_021300 |
| Neosartorya fischeri | NFIA_098820 |
| Neurospora crassa | NCU04691.4 |
| Neurospora crassa | NCU11405.4 |
| Neurospora crassa | NCU08473.4 |
| Neurospora crassa | NCU06010.4 |
| Sclerotinia sclerotiorum | SS1G_09861.1 |
| Sclerotinia sclerotiorum | SS1G_05784.1 |
| Sclerotinia sclerotiorum | SS1G_00516.1 |
| Sclerotinia sclerotiorum | SS1G_01494.1 |
| Sclerotinia sclerotiorum | SS1G_11765.1 |
| Sclerotinia sclerotiorum | SS1G_00517.1 |
| Sclerotinia sclerotiorum | SS1G_14067.1 |
| Sclerotinia sclerotiorum | SS1G_05669.1 |
| Sclerotinia sclerotiorum | SS1G_09125.1 |
| Sclerotinia sclerotiorum | SS1G_02347 |
| Puccinia graminis | PGTG_00147.2 |

It should be noted that a signal peptide region observed in the full-length amino acid sequence of wild-type α-1,3-glucanase is not essential for α-1,3-glucanase activity, in general. Accordingly, a polypeptide derived from various types of known full-length wild-type α-1,3-glucanases by deletion of a signal peptide and a polypeptide derived from the former polypeptide by addition of methionine to the N-terminus thereof are within the scope of the wild-type α-1,3-glucanase in the present invention. In the case of α-1,3-glucanase of Bacillus circulans KA304 as shown in SEQ ID NO: 31 described above, specifically, a polypeptide comprising the amino acid sequence shown in SEQ ID NO: 32 resulting from removal of N-terminal 34 amino acids (i.e., MRTKYVAWSL IAALLITTLF QSVGPGEPVE AAGG) corresponding to the signal peptide region and addition of methionine to the N-terminus from which such 34 amino acids have been removed is within the scope of wild-type α-1,3-glucanase of Bacillus circulans KA304, for example. In addition, a polynucleotide comprising the nucleotide sequence shown in SEQ ID NO: 33 that encodes the aforementioned polypeptide is within the scope of the wild-type α-1,3-glucanase gene of Bacillus circulans KA304.

In the present invention, the term “α-1,3-glucanase variant” refers to a polypeptide comprising an amino acid sequence derived from the amino acid sequence constituting the aforementioned wild-type α-1,3-glucanase by deletion, substitution, and/or addition of 1 or several amino acids or an amino acid sequence having 95% or higher, preferably 98% or higher, and more preferably 99% or higher identity with the former amino acid sequence and having α-1,3-glucanase activity. The term “identity” used herein refers to the percentage (%) of the number of identical amino acid residues of an amino acid sequence relative to the total number of amino acid residues of the other amino acid sequence, including the number of gaps, when two amino acid sequences are aligned to achieve the highest consistency with or without the introduction of gaps thereinto. The term “several” refers to an integer from 2 to 10, such as 2 to 7, 2 to 5, 2 to 4, or 2 or 3. Specific examples of the α-1,3-glucanase variants include naturally-occurring variants, such as variants resulting from polymorphisms (e.g., single nucleotide polymorphisms (SNPs)) or splice variants, and artificial variants having α-1,3-glucanase activity resulting from mutagenesis with the use of a mutagen. Substitution mentioned above is preferably conservative amino acid substitution because a polypeptide comprising an amino acid sequence resulting from conservative amino acid substitution can have substantially equivalent constitution or properties with wild-type α-1,3-glucanase. Examples of conservative amino acids include: non-polar amino acids (glycine, alanine, phenylalanine, valine, leucine, isoleucine, methionine, proline, and tryptophan) and polar amino acids (amino acids other than the non-polar amino acids); charged amino acids (acidic amino acids (aspartic acid and glutamic acid) and basic amino acids (arginine, histidine, and lysine)); uncharged amino acids (amino acids other than the charged amino acids) and aromatic amino acids (phenylalanine, tryptophan, and tyrosine); and branched amino acids (leucine, isoleucine, and valine) and aliphatic amino acids (glycine, alanine, leucine, isoleucine, and valine).

The term “active fragment thereof” used in the present invention refers to a polypeptide comprising wild-type α-1,3-glucanase having α-1,3-glucanase activity or part of the α-1,3-glucanase variant. The length of an amino acid sequence of a polypeptide constituting the active fragment is not particularly limited, provided that the polypeptide has α-1,3-glucanase activity.

α-1,3-glucanase used in the present invention can include any (poly)peptide. Examples thereof include extracellular secretion signal peptides and tag peptides. The aforementioned organism species may be any organism species having the endogenous α-1,3-glucanase gene (i.e., the AGL gene). For example, various bacteria of the genus Bacillus (e.g., Paenibacillus sp. and Geobacillus sp.) and bacteria of the genus Streptomyces may be used. Examples of filamentous fungi include Magnaporthe grisea (oryzae), Aspergillus sp, Sclerotinia sclerotiorum, Neurospora crassa, Botryotinia fuckeliana, Podospora anserine, Neosartorya fischeri, Chaetomium globosum, Penicillium chrysogenum, Penicillium marneffei, Penicillium funiculosum, Talaromyces stipitatus, Talaromyces stipitatus, Schizosaccharomyces pombe, Schizosaccharomyces japonicus, Cryptococcus neoformans, and Hypocrea lixii (Trichoderma harzianum). Filamentous fungi or bacteria of the genera Aspergillus, Penicillium, Schizosaccharomyces, Paenibacillus, and Trichoderma are highly applicable. Applicability of microorganisms of the genera Bacillus, Paenibacillus, Trichoderma, and Aspergillus is particularly high for microbial pesticide formulations. Genes of food microorganisms of the genera Bacillus, Aspergillus (Aspergillus oryzae in particular), and Schizosaccharomyces (Schizosaccharomyces pombe in particular) are more preferably used for recombinant crops.

### 1-2. Method

The methods for preventing or inhibiting infection with plant-infecting microorganisms of the present invention include (1) a method of bringing α-1,3-glucanase into contact with a host plant, (2) a method of expressing a foreign α-1,3-glucanase gene in a host plant cell, (3) a method of allowing a microbial pesticide formulation comprising, as an active ingredient, a microorganism that has the α-1,3-glucanase gene and secretes α-1,3-glucanase to the outside of the cell to act on a host cell, and a method involving the performance of methods (1) to (3) in combination. Hereafter, the methods (1) to (3) are described in detail.

(1) Method of Bringing α-1,3-Glucanase into Contact with Host Plant

This method comprises bringing the pesticide formulation comprising, as an active ingredient, α-1,3-glucanase into contact with a host plant to be protected.

α-1,3-glucanase of the pesticide formulation used in this method can be purified or prepared from an organism species having the endogenous α-1,3-glucanase gene or a transgenic organism species into which the α-1,3-glucanase gene has been introduced in accordance with a method known in the art. For example, such process may be performed in accordance with the method described in Sambrook, J. et al., 1989, Molecular Cloning: A Laboratory Manual Second Ed., Cold Spring Harbor Laboratory Press, Cold Spring Harbor, N.Y.

The pesticide formulation used in this method may be in any form, provided that it can sustain the enzyme activity after α-1,3-glucanase is brought into contact with a host plant. For example, it may be in the liquid form comprising α-1,3-glucanase suspended in an adequate solution or it may be in a solid form, including the form of powder.

When the pesticide formulation is in the liquid form, a solution in which α-1,3-glucanase is suspended is, for example, an aqueous solution, or preferably a buffer. The buffer having a pH value around the optimal pH value of α-1,3-glucanase (i.e., 3.5 to 7.5) and salt concentration around the optimal salt concentration (i.e., 50 mM to 200 mM NaCl) is preferable. Also, carriers that are acceptable for a pesticide formulation can be added to such suspension at a concentration at which α-1,3-glucanase activity is not adversely affected. α-1,3-glucanase concentration in the solution may be between 50 ng/ml and 100 μg/ml, and preferably between 100 ng/ml and 50 μg/ml or between 300 ng/ml and 5 μg/ml, when it is brought into contact with a host plant.

When the pesticide formulation is in the solid form, α-1,3-glucanase prepared via lyophilization is preferable. α-1,3-glucanase in a solid state may be mixed with carriers that are acceptable for a pesticide formulation, as long as it does not inhibit or suppress the enzyme activity.

Examples of “carriers that are acceptable for a pesticide formulation” include excipients, stabilizers, binders, and/or disintegrators.

Examples of excipients include sugars (including glucose, sucrose, lactose, raffinose, mannitol, sorbitol, inositol, dextrin, maltodextrin, starch, and cellulose), metal salts (e.g., sodium phosphate or calcium phosphate, calcium sulfate, and magnesium sulfate), citric acid, tartaric acid, glycine, low-, middle-, or high-molecular weight polyethylene glycol (PEG), and a combination of any thereof. An example of a stabilizer is glycerol. Examples of binders include starch, gelatin, tragacanth, methylcellulose, hydroxypropyl methylcellulose, carboxymethylcellulose sodium, and/or polyvinyl pyrrolidone. Examples of disintegrators include starch mentioned above, carboxymethyl starch, cross-linked polyvinyl pyrrolidone, agar, alginic acid or sodium alginate, and a salt of any thereof. In addition to the substances mentioned above, a diluent, absorbent, emulsifier, solubilizer, moisturizer, antiseptic, antioxidant, buffer, or the like can be added as necessary.

Such carriers are used to stably sustain α-1,3-glucanase activity, facilitate contact with a host plant, and prevent α-1,3-glucanase from being easily removed from a host plant due to weather or other conditions. Thus, such carriers may be adequately used as necessary.

α-1,3-glucanase may be brought into contact with a host plant by any method without particular limitation, provided that α-1,3-glucanase is capable of exerting its enzyme activity in the body of a host plant, and particularly on the surface thereof. Examples of such method include spraying, dispersion, coating, and soaking. α-1,3-glucanase may be brought into contact with part of or the entire body of the host plant. It is preferable that α-1,3-glucanase be brought into contact with the host plant at a site where the largest number of plant-infecting microorganisms to be controlled are observed in the route through which the microorganisms infect the host plant. When rice blast fungus are to be controlled, for example, α-1,3-glucanase may be brought into contact with leaves and stems.

### (2) Method of Expressing Foreign α-1,3-Glucanase Gene in Host Plant Cell

This method comprises preparing a transgenic plant by introducing the α-1,3-glucanase gene into a host plant, expressing the foreign α-1,3-glucanase gene, and preventing or inhibiting infection of a host plant with plant-infecting microorganisms with the aid of α-1,3-glucanase secreted by the transgenic host plant.

This method is advantageous in that continuous effects of infection prevention can be attained without treating a host plant with α-1,3-glucanase each time. In this method, plants comprising plant tissue or cells derived from transgenic plants, seeds thereof, or progenies thereof can also be used.

The α-1,3-glucanase gene used for transforming a host plant is a polynucleotide encoding α-1,3-glucanase mentioned above (i.e., wild-type α-1,3-glucanase, a variant thereof, or an active fragment thereof). Accordingly, it is not always necessary that it comprises a full-length wild-type polynucleotide. Such α-1,3-glucanase gene can be obtained via cloning or chemical synthesis based on the wild-type α-1,3-glucanase gene sequence of various organism species available from the GenBank in accordance with a conventional technique. The α-1,3-glucanase gene may be cloned in accordance with the method described in, for example, Sambrook, J. et. al., 1989, Molecular Cloning: A Laboratory Manual Second Ed., Cold Spring Harbor Laboratory Press, Cold Spring Harbor, N.Y.

The expression vector of the present invention comprises an expression promoter such that the α-1,3-glucanase gene is expressed in the plant body when it is introduced into the host plant. In general, the α-1,3-glucanase gene is located at a site downstream of such promoter, and a terminator is located at a site downstream of such gene. A vector used for this purpose is adequately selected by a person skilled in the art in accordance with a method of introduction thereof into a plant or a plant type. Examples of such promoters include the cauliflower mosaic virus (CaMV)-derived 35S promoter, the maize ubiquitin promoter, and the EN4 promoter. In order to enhance the expression efficiency, for example, a promoter comprising a TMΩ sequence or the like, such as a E12Ω promoter, can be used. Examples of the terminators include the cauliflower mosaic virus-derived terminator and the nopaline synthase gene-derived terminator. Promoters and terminators are not limited to those exemplified above, provided that they function in host plant cells.

In order to efficiently select transgenic plant cells into which the α-1,3-glucanase gene has been introduced, it is preferable that the expression vector comprise an adequate selection marker gene cassette or the expression vector be introduced into plant cells with DNA comprising a selection marker gene cassette. Examples of selection marker genes used for such purpose include, but are not limited to, the hygromycin phosphotransferase gene providing resistance to the antibiotic hygromycin and the neomycin phosphotransferase gene providing resistance to kanamycin.

An expression vector comprising a DNA fragment of the α-1,3-glucanase gene or the α-1,3-glucanase gene can be introduced into host plant cells by a method known in the art, such as the agrobacterium method, electroporation, the particle gun method, or the polyethylene glycol. Plant cells into which the α-1,3-glucanase gene has been introduced are efficiently selected via culture under adequate conditions, in accordance with a type of the selection marker gene introduced.

A plant body can be reproduced from a transgenic cell into which the α-1,3-glucanase gene has been introduced. A plant body can be reproduced by a method known in the art in accordance with plant cell type and the method of gene introduction employed. When the gene is introduced into a callus by the agrobacterium method, for example, a plant body can be reproduced from the callus (Toki, et al., Plant Journal, 47, 969-976, 2006). When electroporation is employed, a plant body can be reproduced from a protoplast (Toki, et al., Plant Physiol., 100, 1503-1507, 1992). Once transgenic plant cells or seeds into which the α-1,3-glucanase gene has been introduced in the genome are obtained, cultured plant tissue or plant bodies can be mass-produced with the use of such cells or seeds.

Whether or not the resulting transgenic plants are resistant to infection with plant-infecting microorganisms can be confirmed by, for example, bringing the microorganisms (e.g., spores or hyphae) into contact with transgenic plants under conditions in which microorganisms to be controlled easily infect plants and inspecting whether or not such microorganisms infect the plants. The microorganisms are brought into contact with the transgenic plants by, for example, a method in which plant bodies are sprayed with a suspension of microorganisms to be controlled, cultured, and then observed (may be referred to as “spray inoculation”), a method in which plant bodies are wounded with a puncher, agarose slices comprising the microorganisms to be controlled are placed on the wounds, and plant bodies are observed after culture (may be referred to as “wound inoculation”), or a method in which a suspension of microorganisms to be controlled is applied to a needle tip and the plant bodies are damaged with a needle (may be referred to as “needle inoculation”).

(3) Method of Allowing a Microbial Pesticide Formulation Comprising, as an Active Ingredient, a Microorganism that has the α-1,3-Glucanase Gene and Secretes α-1,3-Glucanase to the Outside of the Cell to Act on a Host Cell

This method comprises bringing a microbial pesticide formulation comprising, as an active ingredient, a microorganism capable of biosynthesizing α-1,3-glucanase into contact with a host plant and preventing or inhibiting the host plant from being infected with plant-infecting microorganisms by the action of α-1,3-glucanase secreted by the microorganisms to the outside of the cells.

This method is advantageous in that effects can be more prolonged compared with the effects attained by a method involving contact with an enzyme, preparation of transgenic plants is not necessary, and processes are simple.

The microbial pesticide formulation used in this method may be the microbial pesticide formulation described in the second aspect described below.

The method of allowing the microbial pesticide formulation to act on host plants is not particularly limited, provided that microorganisms as active ingredients of the microbial pesticide formulation used in this method are capable of exerting the effects of the present invention by expressing α-1,3-glucanase at a significantly higher level than the case of normal growth of wild-type strains and secreting α-1,3-glucanase to the outside of the cells.

The term “significant” used herein refers to the situation in which there are significant differences in the statistically processed quantities between the α-1,3-glucanase expression level in the microorganisms as active ingredients and the α-1,3-glucanase expression level when the wild-type strains of the microorganism grow under normal conditions (i.e., the microorganisms grow under optimal conditions in terms of adequate nutritional conditions, growth temperature, pH levels, and concentration). Specific examples include cases in which a critical rate (i.e., a significant standard) is smaller than 5%, 1%, or 0.1%. Known testing methods capable of determining the significance may be adequately employed as the test methods for statistical processing, and such methods are not particularly limited. For example, the student's t test or multiple comparison test may be employed. The term “significantly high” refers to the situation in which the α-1,3-glucanase expression level of microorganisms as active ingredients is significantly higher than that of wild-type strains. Specifically, the expression level of interest is 1.5 times or higher, and preferably 2 or 3 times higher than the α-1,3-glucanase expression level of wild-type strains at a normal state, for example.

When the microbial pesticide formulation is allowed to act on host plants, accordingly, the method therefore may be adequately determined by taking the conditions of the α-1,3-glucanase gene of the microorganisms as active ingredients into consideration, so that the α-1,3-glucanase expression level becomes significantly higher than the expression level attained when wild-type strains grow under normal conditions. When the microorganisms as active ingredients have an expression vector capable of constitutive expression of the α-1,3-glucanase gene in the cells, for example, a microbial pesticide formulation comprising such microorganisms may be brought into contact with host plants. When the microorganisms comprise the α-1,3-glucanase gene ligated to an inducible promoter (e.g., many endogenous α-1,3-glucanase genes or foreign α-1,3-glucanase genes ligated to a lac promoter in an expressible manner or the like), the α-1,3-glucanase genes of the microorganisms may be induced and accelerated to express before or after the microbial pesticide formulation is brought into contact with host plants. Specifically, a substance capable of inducing and accelerating expression (i.e., an expression inducer) may be directly added to the microbial pesticide formulation, or a microbial pesticide formulation is brought into contact with host plants and then separately added to the microbial pesticide formulation. An expression inducer may be adequately selected in accordance with promoter type. In the case of the endogenous α-1,3-glucanase gene promoter, α-1,3-glucan which is a substrate of such enzyme can be used. In such a case, accordingly, a substance that comprises α-1,3-glucan and does not impair α-1,3-glucanase activity may be added, in addition to α-1,3-glucan (e.g., purified and/or unpurified α-1,3-glucan). When the microorganisms comprise the α-1,3-glucanase gene ligated to the lac promoter, lactose or a substance that comprises lactose and does not impair α-1,3-glucanase activity can be used.

Specifically, the microbial pesticide formulation is allowed to act on host plants via contact or absorption through roots, for example. In general, a method involving contact is preferable. Such method can be carried out in accordance with the contact method described in (1) Method of bringing α-1,3-glucanase into contact with a host plant.

### 1-3. Effects

According to the present invention, cell-wall α-1,3-glucan on the infectious hyphae of plant-infecting microorganisms including filamentous fungi having α-1,3-glucan on the cell wall, such as the rice blast fungus, is degraded during/upon infection by allowing α-1,3-glucanase to be present on the surface or in the tissues of the host plants. Once α-1,3-glucan is degraded, the covered chitin and β-1,3-glucan are exposed, and host plants can recognize the microorganisms. This can excite the defense mechanisms in host cells and fungal infection can thus be inhibited.

Accordingly, the concept of the method for preventing or inhibiting infection of the present invention is fundamentally different from that of conventional methods for preventing infection in which the β-1,3-glucanase or chitinase gene or protein is introduced to directly attack hyphae. According to conventional techniques, specifically, such enzymes attack hyphae. In contrast, the method of the present invention is intended to promote the inherent defense mechanisms of plants to plant-infecting microorganisms on which β-1,3-glucanase or chitinase would not act very effectively, for example, the microorganisms covered with α-1,3-glucan. Accordingly, the method of the present invention is based on a novel idea.

### 2. Microbial Pesticide Formulation

### 2-1. Constitution

The second aspect of the present invention relates to a microbial pesticide formulation, which prevents or inhibits infection of plants with plant-infecting microorganisms. The microbial pesticide formulation of the present invention comprises, as an active ingredient, a microorganism that has the α-1,3-glucanase gene and secretes α-1,3-glucanase to the outside of the cell.

The microorganism as an active ingredient of the present invention is not particularly limited, provided that it has the α-1,3-glucanase gene and is capable of secreting the expressed α-1,3-glucanase to the outside of the cell. Examples include infectious and non-infectious microorganisms.

The term “infectious microorganisms” used herein refers to microorganisms that are pathogenic and infectious to other organisms. Examples thereof include bacteria, yeast, filamentous fungi (including molds), and basidiomycetes (e.g., mushrooms). When infectious microorganisms are used in the present invention, use of microorganisms lacking the pathogenicity or having pathogenicity attenuated to the extent that the microorganisms are not harmful on the organisms is preferable from the viewpoint of safety on plants and/or mammalians to be protected. However, microorganisms may be harmful and infectious to pests, such as aphids, scale insects, planthoppers, leafhoppers, lace bugs, locusts, moths (e.g., larvae of Mamestra), or mites because such properties are useful for the active ingredient of the pesticide formulation to pests.

The term “non-infectious microorganisms” refers to microorganisms that are not pathogenic or infectious to at least plants to be protected by the present invention. When such plants are used for food products, the term refers to microorganisms that are not infectious to mammalians, including humans, such as bacteria, yeast, filamentous fungi (including molds), or basidiomycetes (e.g., mushrooms). Preferably, non-infectious microorganisms have the α-1,3-glucanase genes as the endogenous genes as described below. Examples of bacteria include those of the genera Bacillus (e.g., Paenibacillus sp. and Geobacillus sp.) and Streptomyces, with Bacillus circulans (Paenibacillus sp.) being particularly preferable. Examples of filamentous fungi include Aspergillus sp, Neurospora crassa, Podospora anserine, Neosartorya fischeri, Chaetomium globosum, Penicillium chrysogenum, Penicillium funiculosum, Schizosaccharomyces pompe, Schizosaccharomyces japonicus, and Hypocrea lixii (Trichoderma harzianum). Microorganisms of the genera Bacillus, Aspergillus, Penicillium, Schizosaccharomyces, Paenibacillus, and Trichoderma are particularly preferable.

The microorganisms as active ingredients of the microbial pesticide formulation of the present invention may comprise either or both the endogenous α-1,3-glucanase gene and the foreign α-1,3-glucanase gene. In the light of dispersing the microbial pesticide formulation in agricultural fields, microorganisms having the endogenous gene are preferable.

The term “α-1,3-glucanase gene” used in the present invention refers to a nucleic acid encoding α-1,3-glucanase described in the first aspect, such as a nucleic acid shown in SEQ ID NO: 23 or the Accession Number XP001410317.

The “α-1,3-glucanase” of the present invention is of an “extracellular secretion type.” The term “extracellular secretion type” refers to the situation in which α-1,3-glucanase that was biosynthesized in microbial cells is secreted to the outside of the cells in the end. As long as α-1,3-glucanase is secreted to the outside of the cells, means therefore are not limited. For example, α-1,3-glucanase may have an extracellular signal peptide, or it may be secreted outside the cells with the aid of other extracellular transport factors.

The microorganisms as active ingredients of the present invention are preferably capable of expressing α-1,3-glucanase at a level significantly higher than the level attained when wild-type strains grow under normal conditions. To this end, the α-1,3-glucanase gene is preferably ligated to a site downstream of a constitutive or inducible promoter in the microorganisms in an expressible manner. An example of a constitutive promoter is S10 promoter, and examples of inducible promoters include lac and trp promoters and a promoter inherent to the endogenous α-1,3-glucanase gene.

Microorganisms as active ingredients of the microbial pesticide formulation of the present invention may have or have not expressed α-1,3-glucanase when allowed to act on host plants. When microorganisms as active ingredients constitutively express α-1,3-glucanase or have expressed α-1,3-glucanase, which has been induced and accelerated to express via induction treatment, the microorganisms as active ingredients may be alive or dead in the microbial pesticide formulation, provided that α-1,3-glucanase is stably maintained. If α-1,3-glucanase has not yet been expressed, it is induced to express after the microbial pesticide formulation is brought into contact with host plants as described above. In such a case, accordingly, the microorganisms as active ingredients are required to be alive until they act on host plants. The “microbial pesticide formulation” of the present invention may be in the state of a liquid, solid (including semi-solid), or a combination thereof.

When the microbial pesticide formulation is in a liquid state, microorganisms as active ingredients may be suspended in an adequate solution. Examples of adequate solutions include buffer and a medium used for relevant microorganisms. Carriers that are acceptable for a pesticide formulation can be added to the suspension of microorganisms at a concentration at which such carriers would not block α-1,3-glucanase activity. The carriers that are acceptable for a pesticide formulation described in 1-2. Method of the first aspect, (1) Method of bringing α-1,3-glucanase into contact with host plant may be used.

An adequate expression inducer that is effective for α-1,3-glucanase expression can be added to the solution as necessary. An expression inducer may be adequately selected in accordance with properties of the α-1,3-glucanase gene promoter of the microorganisms as active ingredients, as described in 1-2. Method of the first aspect, (3) Method of allowing a microbial pesticide formulation comprising, as an active ingredient, a microorganism that has the α-1,3-glucanase gene and secretes α-1,3-glucanase to the outside of the cell to act on a host cell. If the promoter is inherent to the endogenous α-1,3-glucanase gene, for example, use of α-1,3-glucan as the substrate is adequate. In the case of a lac promoter, use of lactose as the substrate is adequate. An adequate volume of such expression inducer may be added in accordance with expression induction conditions.

When the microbial pesticide formulation is in a solid state, the form thereof is not particularly limited, provided that microorganisms as active ingredients, more specifically α-1,3-glucanase synthesized by such microorganisms, are capable of acting on host plants. Examples thereof include granule state, powder state, and semi-solid states such as gel state. In the light of the microbial pesticide formulation adheres to host plants via contact or other means and acts thereon, the state of powder (an adhesive powder state, in particular), or gel is preferable.

### 2-2. Effects

The microbial pesticide formulation of the present invention is extensively effective for the control of infection of plants with plant-infecting microorganisms having α-1,3-glucan. In addition, the microbial pesticide formulation of the present invention can be produced in a relatively cost-effective manner. When non-infectious microorganisms having the endogenous α-1,3-glucanase gene are used as active ingredients, also, microorganisms existing in nature in which expression of given genes is reinforced are used. Thus, the influence of the microbial pesticide formulation of the present invention on the environment is small, and safety thereof is satisfactory.

### EXAMPLE 1

### Detection of Cell Wall Constituent in Infection Structure

The spore (conidial) suspension of rice blast fungus (1×10⁶ conidiospores per ml of sterile water, 50 μl) was injected into the leaf sheath cells at the 4th node of the rice variety (LTH) susceptible to the wild-type rice blast fungus (Guy11) using a syringe, and the resultant was allowed to stand at room temperature. After inoculation, appressorium formation and infectious hyphae formation were observed in the germinated conidiospores approximately 16 hours and 24 hours after inoculation, respectively.

The rice leaf sheath infected with microorganisms were fixed via 3% (v/v) formaldehyde/90% (v/v) ethanol immersion 16 hours and 24 hours after inoculation, respectively, and foliar tissue was extracted and thoroughly rinsed in PBS buffer (137 mM NaCl, 2.7 mM KCl, 8.1 mM Na₂HPO₄, 1.5 mM KH₂PO₄, pH 7.4). The fixed leaf sheath samples were immersed in 1% (v/v) Tween 20 (in PBS buffer; may be referred to as “PBS-T”), a reagent capable of specifically staining a relevant cell wall component was added to 1% (v/v) Tween 20 (in PBS buffer), and the samples were stained in the manner as described in A to D below.

### A. α-1,3-Glucan Staining

The α-1,3-glucan-specific mouse IgM antibody (0.1 mg/ml, 20 μl; tradename: Mouse IgMλ (MOPC104e), α-1→3-glucan-specific, Sigma) was added and the resultant was incubated overnight. Subsequently, the Alexa Fluor 488 labeled anti-mouse IgM antibody (0.1 mg/ml, 20 μl; tradename: Alexa Fluor 488 goat anti-mouse IgM, Invitrogen) was added and the resultant was incubated under light-shielded conditions overnight. B. β-1,3-Glucan staining

The β-1,3-glucan-specific mouse monoclonal antibody (0.1 mg/ml, 20 μl; tradename: Monoclonal antibody to (1→3)-β-glucan (Mouse IgG Kappa Light, Biosupplies) was added and the resultant was incubated overnight. Subsequently, the Alexa Fluor 594 labeled anti-mouse IgG antibody (0.15 mg/ml, 20 μl; tradename: Alexa Fluor 594 goat anti-mouse IgG (H+L) antibody, Invitrogen) was added and the resultant was incubated under light-shielded conditions overnight.

### C. Chitin Staining

The Alexa Fluor 350 labeled WGA (10 μg/ml, 20 μl, wheat germ agglutinin, tradename: Alexa Fluor 350 conjugate, Invitrogen) was added and the resultant was incubated under light-shielded conditions overnight.

### D. Chitosan Staining

Eosin (0.05% (w/v), 20 μl; tradename: Eosin Y, Sigma) was added and the resultant was incubated under light-shielded conditions overnight.

### E. Mannan Staining

FITC labeled concanavalinA (0.1 mg/ml, 20 μl; tradename: FITC conjugated concanavalin A, Sigma) was added and the resultant was incubated under light-shielded conditions overnight.

Excess amounts of staining reagents remaining in the incubated samples were thoroughly removed via rinsing with PBS and the samples were subjected to fluorescence microscope observation. Microscopic observation was carried out with the use of the Leica DR System (Leica, Germany).

As fluorescent filters, the GFP Filter Cube (excitation filter BP 470/40 nm, 500 nm dichromatic mirror, suppression filter BP 525/50 nm) was used for fluorescent observation of α-1,3-glucan and mannan, the Y3 Filter Cube (excitation filter BP 545/30 nm, 565 nm dichromatic mirror, suppression filter BP610/75 nm) was used for the β-1,3-glucan stain sample and the chitosan stain sample, and the A4 Filter Cube (excitation filter BP 360/40 nm, 400 nm dichromatic mirror, suppression filter BP 470/40 nm) was used for the chitin stain sample.

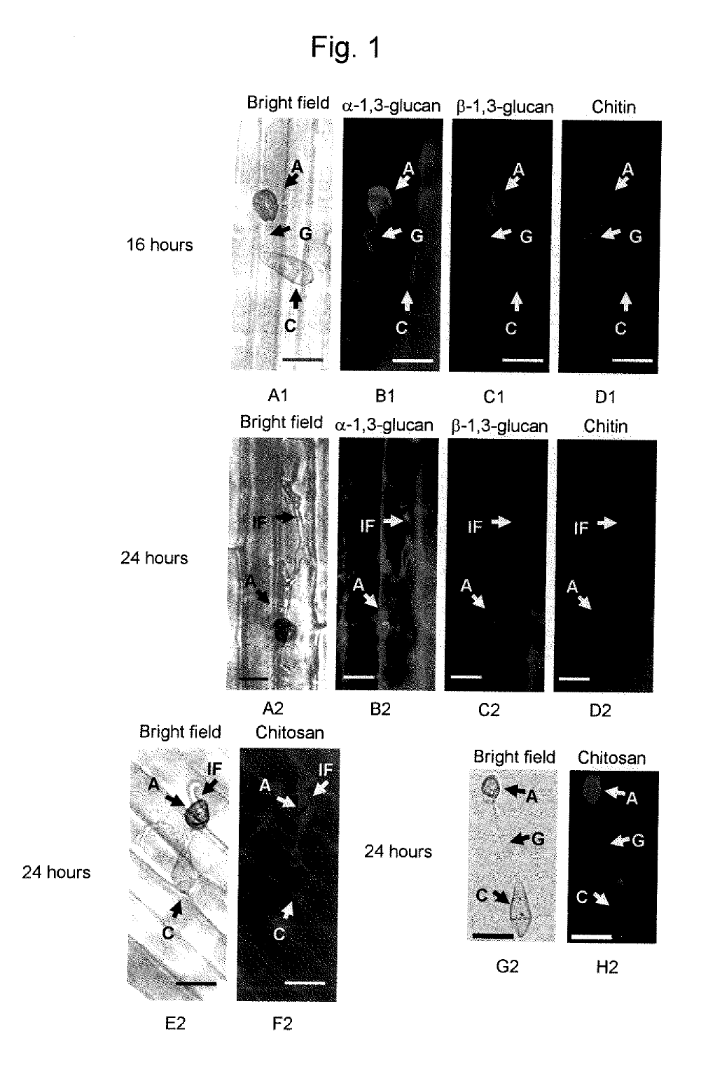

The results are shown in FIG. 1. In the figure, “C” represents a spore, “G” represents a germ tube, “A” represents an appressorium, “IF” represents an infectious hyphae, and each bar in the panels represents 20 μm.

α-1,3-Glucan was detected in the germ tube and the immature appressorium 16 hours after inoculation of plant cells with rice blast fungus (panel B1), and it was detected in the infectious hyphae 24 hours after inoculation (panel B2). While an insignificant amount of β-1,3-glucan was detected in the immature appressorium 16 hours after inoculation (panel C1), it was not detected in any fungal organs 24 hours later (panel C2). While chitin was detected in the germ tube and the immature appressorium 16 hours after inoculation (panel D1), it was not detected in any fungal organs 24 hours after inoculation (panel D2). Chitosan was detected in both the appressorium and the infectious hyphae 24 hours after inoculation (panel F2). Mannan was detected in the spore, the germ tube, and the appressorium (panel H2).

This demonstrates that α-1,3-glucan and chitosan, among cell wall components such as α-1,3-glucan, β-1,3-glucan, mannan, chitin, and chitosan, are mainly detected in the infectious hyphae of rice blast fungus and β-1,3-glucan and chitin become undetectable in an organ-specific manner.

### EXAMPLE 2

### Detection of Cell Wall Component in Infectious Hyphae After α-1,3-Glucanase Treatment

In the same manner as described above, the leaf sheath cells of the rice variety (LTH) were infected with rice blast fungus, and the rice leaf sheathes infected with the microorganisms were fixed 24 hours after inoculation. Thereafter, 30 μl of a Bacillus-circulans-derived purified α-1,3-glucanase solution (5 μg/ml) was added to PBS buffer used for soaking, and the mixture was incubated at room temperature for 6 hours. Thereafter, the resultant was thoroughly washed with PBS buffer, and cell wall components were stained in the same manner as described above.

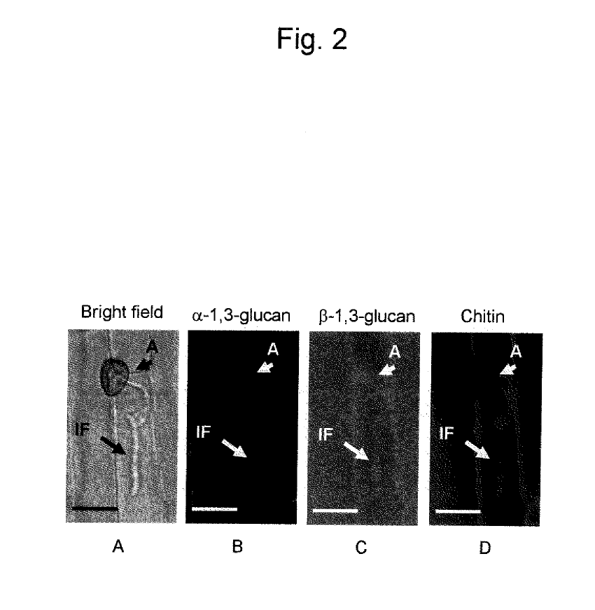

The results are shown in FIG. 2. In the infectious hyphae treated with α-1,3-glucanase, α-1,3-glucan was degraded by α-1,3-glucanase, and it was thus not detected (panel B). In contrast, β-1,3-glucan and chitin, which were not detected before enzyme treatment, became detectable in the infectious hyphae (panels C and D),

This demonstrates that β-1,3-glucan and chitin are present as cell wall components in the infectious hyphae of rice blast fungus and they are covered with α-1,3-glucan.

### EXAMPLE 3

### Infectivity of α-1,3-Glucanase-Deficient Fungal Rice Blast Pathogen Strain (ΔMgAGS1)

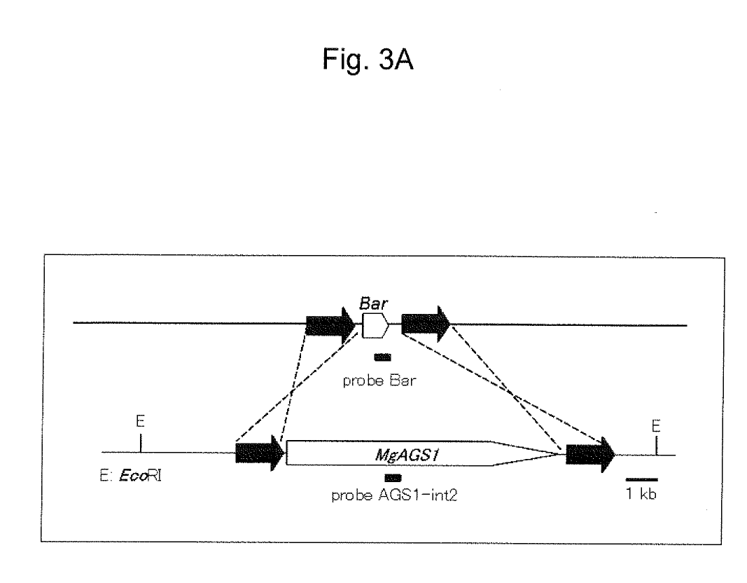

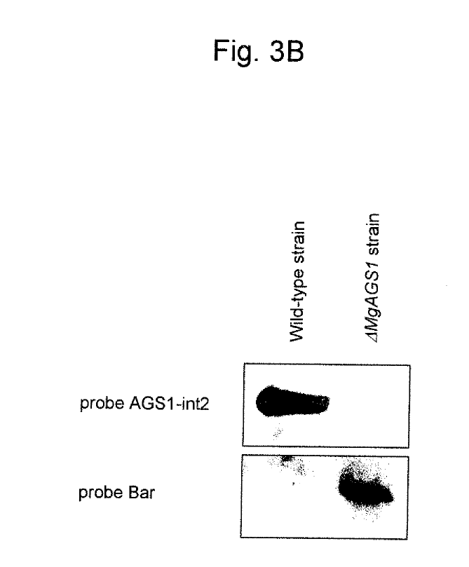

A genome fragment containing the α-1,3-glucan synthase gene (MgAGS1) of rice blast fungus was cloned, the coding region of MgAGS1 was substituted with a drug-tolerance marker (i.e., the bialaphos resistance gene (the Bar gene)), and the resultant was introduced into the wild-type fungal rice blast pathogen strain to prepare the MgAGS1 gene-deficient strain (ΔMgAGS1) (FIG. 3A).

Specifically, a region of approximately 1.5 kb (SEQ ID NO: 1) and a region of approximately 1.5 kb (SEQ ID NO: 2) located upstream and downstream of MgAGS1 (GenBank: XP 364794) were cloned, respectively (SEQ ID NOs: 3 to 6 were used for primers), and the upstream region, the Bar gene (the marker, SEQ ID NO: 7), and the downstream region were ligated in that order via PCR to prepare fusion DNA. The fusion DNA was further amplified via PCR (SEQ ID NOs: 4 and 5 were used for primers), and the amplified DNA fragment was transformed into the wild-type fungal rice blast pathogen strain via the protoplast-PEG method. Transformation of rice blast fungus was carried out in the following manner. The rice blast fungus (hyphae) were immersed in L2 M sorbitol containing lytic enzymes at 30 μg/ml (Sigma lysing enzyme) to prepare protoplasts, and the DNA fragment amplified above was added together with PEG buffer (40% (W/V) PEG 8000, 20% (W/V) sucrose, 50 mM CaCl₂, pH 8.0) to perform gene introduction. Thereafter, the resultant was allowed to grow in a growth medium containing bialaphos (90 μg/ml), and the transformants were selected. The transformants that had been confirmed to lack MgAGS1 via Southern hybridization, PCR (primers shown in SEQ ID NOs: 10 to 13 were used), and sequencing were designated as the ΔMgAGS1 strains (FIG. 3B). When lack of gene was confirmed, the AGS1-int2 probe sequence (SEQ ID NO: 15) and the Bar probe sequence (SEQ ID NO: 14) were used as indicators for the MgAGS1 gene and the Bar gene, respectively. In FIG. 3B, the upper panel demonstrates that MgAGS1 is detected with the use of the AGS1-int2 probe in wild-type strains but it is not detected in the ΔMgAGS1 strains. The lower panel demonstrates that the Bar gene is not detected in wild-type strains but is detected in the ΔMgAGS1 strains with the use of the Bar probe.

### A. Capacity of the ΔMgAGS1 Strain for Forming Infection Structure

30 μl of a spore suspension (1×10⁵ conidiospores per ml of sterile water) of wild-type fungal rice blast pathogen strains and the ΔMgAGS1 strains was added dropwise to a cover glass (Matsunami Glass Ind., Ltd., Osaka), the resultant was allowed to stand at room temperature for 24 hours, and the appressorium formed 24 hours later was observed.

The infectious hyphae formed after the aforementioned samples which had been allowed to stand for 24 hours were observed in the same manner as described above, except that onion scale cells boiled in a microwave oven were used instead of a cover glass.

Microscope observation was carried out with the use of the Leica DR System (Leica, Germany).

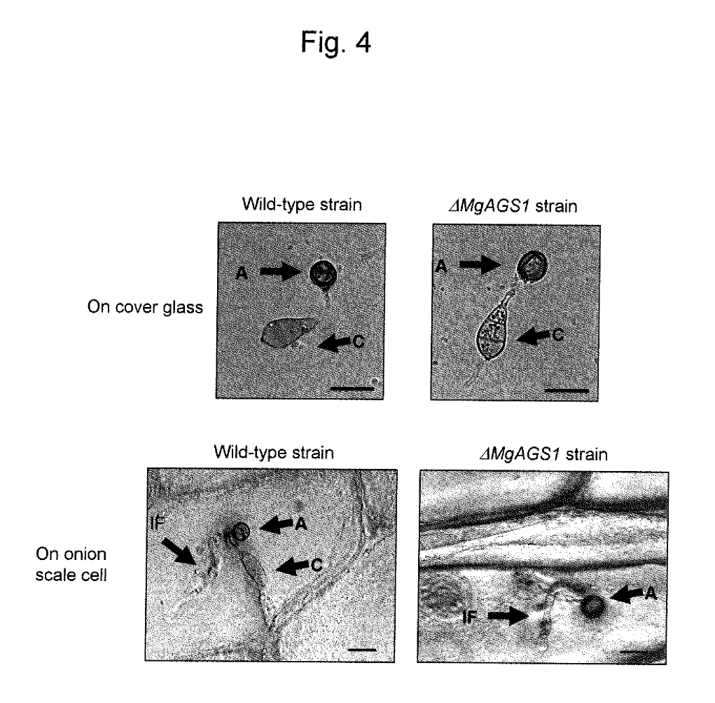

The results are shown in FIG. 4. The ΔMgAGS1 strains exhibited budding of conidiospores and formation of appressorium after they had been allowed to stand for 24 hours, as in the case of the wild-type strains on the cover glass (the upper panel). The ΔMgAGS1 strains also exhibited formation of appressorium and formation of infectious hyphae in the cells, as in the case of the wild-type strains on the onion scale cells (the lower panel). Thus, the ΔMgAGS1 strains maintained a capacity for appressorium and infectious hyphae formation equivalent to that of wild-type strains.

### B. Rice Inoculation Experiment

Cut leaves of the rice variety (LTH; rice that had developed the fourth node was used) were spray-inoculated with 10 ml of a spore suspension of wild-type rice blast fungus and rice blast fungus of the ΔMgAGS1 strains (1×10⁶ conidiospores per ml of sterile water), and the inoculated leaves were incubated under continuous light conditions at 25° C. Lesions that had developed on the cut leaves were observed 5 days after inoculation.

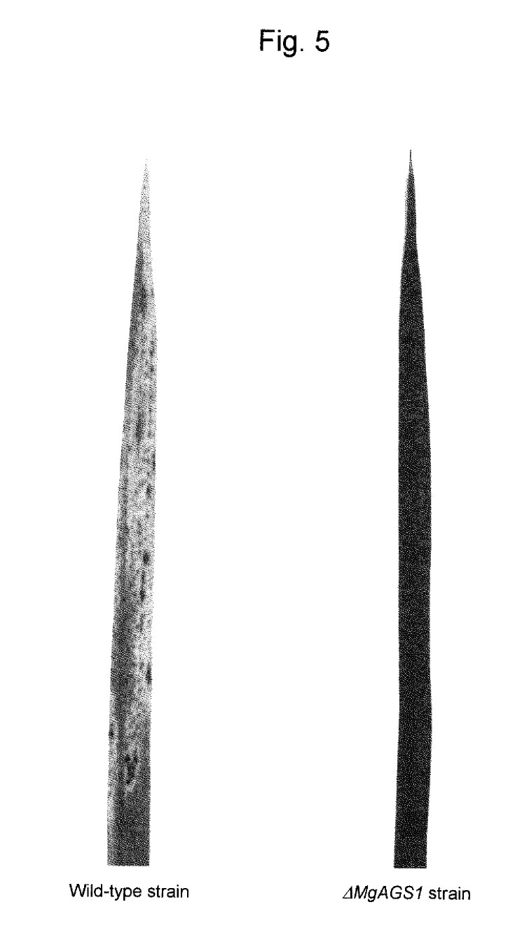

The results are shown in FIG. 5. While developing lesions were observed in rice leaves of wild-type strains (the left panel), substantially no lesions were observed in the ΔMgAGS1 strain, and brown spot lesions observed when rice exhibits resistance responses were observed (the right panel). Accordingly, the ΔMgAGS1 strains were found to have attenuated infectivity against rice.

### C. Barley Inoculation Experiment

Cut leaves of the barley variety (Golden Promise; barley that had developed the fourth leaves was used) were spray-inoculated with 10 ml of a spore suspension of wild-type rice blast fungus and rice blast fungus of the ΔMgAGS1 strains (1×10⁶ conidiospores per ml of sterile water), and the inoculated leaves were incubated under continuous light conditions at 25° C. Lesions that had developed on the cut leaves were observed 5 days after inoculation.

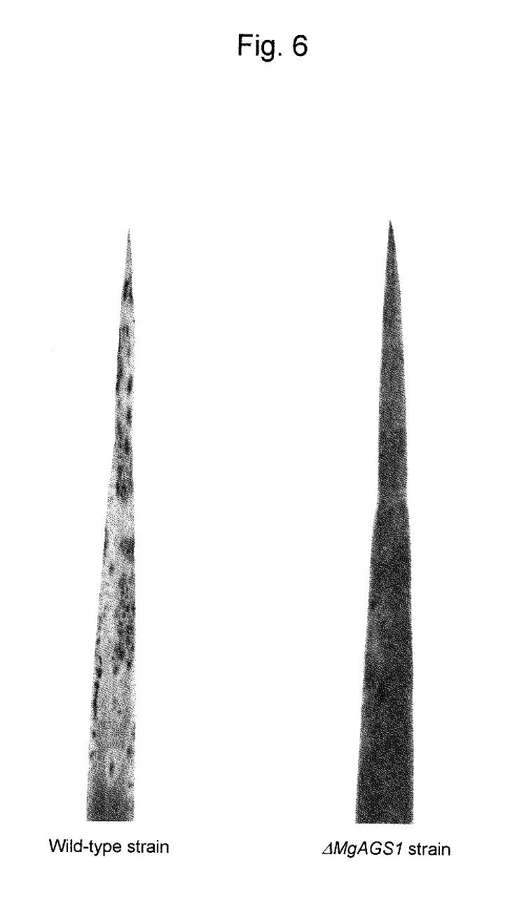

The results are shown in FIG. 6. While developing lesions were observed in barley leaves of wild-type strains (the left panel), the number of lesions was significantly small in the ΔMgAGS1 strains (the right panel). Accordingly, the ΔMgAGS1 strains were found to have attenuated infectivity against barley.

### EXAMPLE 4

### Inhibition of Infectious Hyphae Formation in Plants with the Addition of α-1,3-Glucanase of Rice Blast Fungus

### A. Rice Inoculation Experiment (Microscope Observation)

A purified α-1,3-glucanase solution (5 μg/ml, 0.5 ml) was added to 50 μl of a spore suspension of wild-type rice blast fungus (1×10⁶ conidiospores per ml of sterile water), the leaf sheaths at the fourth node of the rice variety (LTH) were inoculated with the resultant, and the inoculated sheaths were allowed to stand at room temperature. As a control, the solution obtained by adding 0.5 ml of sterile water to the spore suspension was inoculated instead of the α-1,3-glucanase solution, and the inoculated sheaths were allowed to stand in the same manner.

The rice leaf sheaths infected with the fungi were fixed with the use of 3% (v/v) formaldehyde/90% (v/v) ethanol 48 hours after inoculation in the same manner as described above, followed by microscope observation.

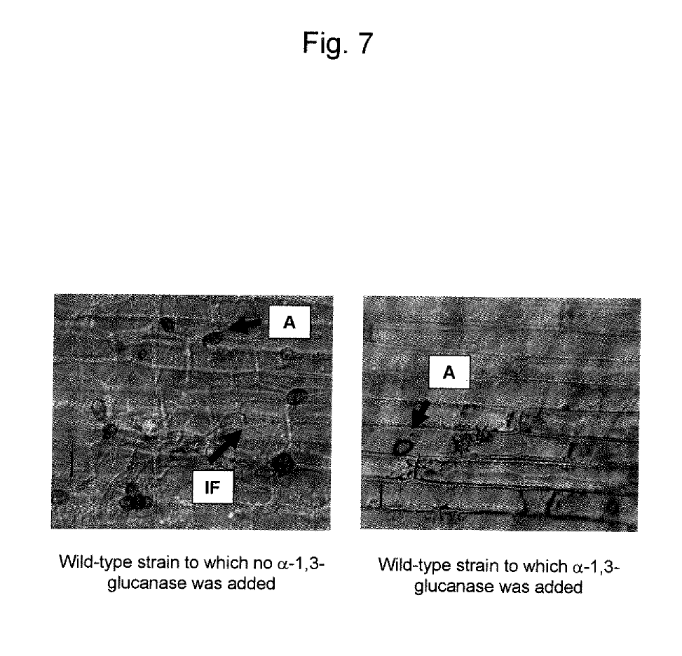

The results are shown in FIG. 7. When the leaf sheaths were inoculated with a spore suspension of wild-type strains to which α-1,3-glucanase had not been added, appressorium and infectious hyphae formations were observed 48 hours after inoculation (the left panel). When the leaf sheaths were inoculated with a spore suspension of wild-type strains to which α-1,3-glucanase had been added, however, the number of appressoriums adhered to the rice cells was small, and the infectious hyphae were not formed (the right panel).

The above results demonstrate that the α-1,3-glucan-defective strains (the ΔMgAGS1 strain) form appressoriums and infectious hyphae on the cover glass or dead plant cells as with the wild-type strains, but they exhibit apparently attenuated infectivity on living plants compared with wild-type strains. The results also demonstrate that infectivity of wild-type strains could be significantly suppressed via treatment with foreign α-1,3-glucanase.

### B. Rice Inoculation Experiment (Visual Observation)

Cut leaves of the rice variety (LTH; rice that had developed the fourth node was used) were spray-inoculated with 10 ml of a spore suspension of wild-type strains to which 0.5 ml of a purified α-1,3-glucanase solution (5 μg/ml) had been added, and the inoculated leaves were incubated under continuous light conditions at 25° C. As a control, the solution obtained by adding 0.5 ml of sterile water to the spore suspension was inoculated instead of the α-1,3-glucanase solution, and incubation was carried out in the same manner. Lesions that had developed on the cut leaves were observed 5 days after inoculation.

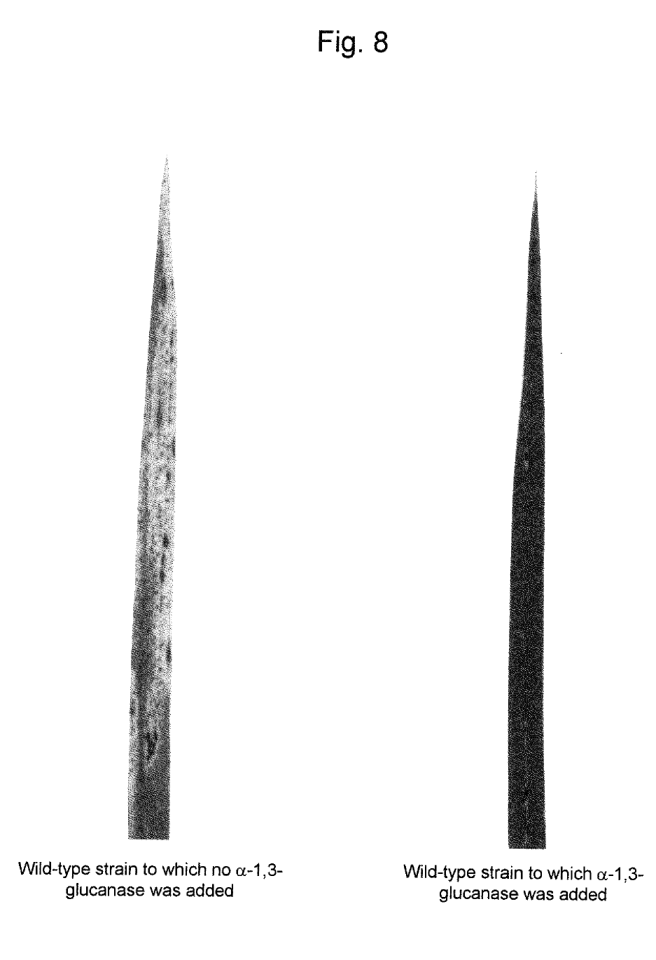

The results are shown in FIG. 8. within the wild-type strains to which no α-1,3-glucanase had been added, developing lesions were formed (the left panel). In the wild-type strains to which α-1,3-glucanase had been added, the number of lesions was significantly reduced (the right panel). Accordingly, it was demonstrated that degradation of α-1,3-glucan on the cell wall of rice blast fungus would lead to the attenuated infectivity of rice blast fungus.

### C. Barley Inoculation Experiment (Visual Observation)

Cut leaves of the barley variety (Golden Promise, the barley that had developed the fourth node was used) were spray-inoculated with 10 ml of a spore suspension of wild-type strains to which 0.5 ml of a purified α-1,3-glucanase solution (5 μg/ml) had been added, and the inoculated leaves were incubated under continuous light conditions at 25° C. As a control, the solution obtained by adding 0.5 ml of sterile water to the spore suspension was inoculated instead of the α-1,3-glucanase solution, and incubation was carried out in the same manner. Lesions that had developed on the cut leaves were observed 5 days after inoculation.

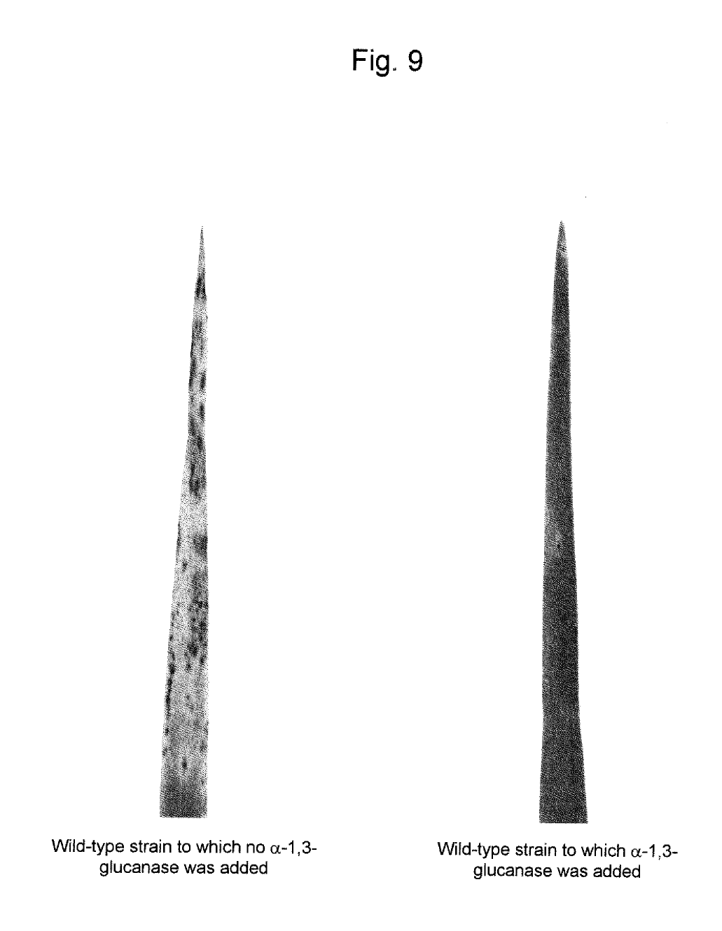

The results are shown in FIG. 9. In the wild-type strains to which no α-1,3-glucanase had been added, developing lesions were formed (the left panel). In the wild-type strains to which α-1,3-glucanase had been added, the number of lesions was significantly reduced (the right panel). Accordingly, it was demonstrated that degradation of α-1,3-glucan on the cell wall of rice blast fungus would lead to the attenuated infectivity of rice blast fungus.

### EXAMPLE 5

### Expression of Cell Wall Polysaccharide Synthase Genes During Appressorium Formation on Plastic Surface

The transcription levels of the α-1,3-glucan synthase gene (MgAGS1) and the β-1,3-glucan synthase gene (MgFKS1) during appressorium formation were inspected via quantitative real-time PCR (qRT-PCR) analysis.

Total RNA was isolated from budding spores or spores developing from the appressorium on the hydrophobic surface of the GelBond film (tradename, Takara) using the QIAGEN Plant mini easy kit (tradename, Qiagen). Fungus body developing on the GelBond surface were collected with the use of a silicone scraper (Toray) and resuspended in RNA later (tradename, Ambion). Total RNA derived from the rice leaf sheath inoculated with fungus spores was isolated with the use of the QIAGEN Plant mini easy kit (tradename, Qiagen). cDNA was synthesized from total RNA samples with the use of the ExScript RT reagent kit (tradename, Takara) and oligo dT primers and used as a template for qRT-PCR. The SYBR Premix ExTaq kit (tradename, Takara) was used for labeling and amplification of template cDNA used for qRT-PCR. Gene-specific primers were designed in order to amplify a unique sequence (approximately 300 bp) of the relevant gene to be used for qRT-PCR (Table 5, SEQ ID NOs: 16 to 21). qRT-PCR analysis was carried out with the use of the Stratagene Mx300p system (tradename, Stratagene) in accordance with the manufacturer's instructions. The transcription levels of these genes were quantified by the delta-Ct method (Livak and Schmittgen, 2001).

TABLE 5

| | | | SEQ |
|---|---|---|---|
| Primer names | | Sequence | ID NO: |
| Primers | Forward | 5′-CCTTTGTCGCGCCGTTTG-3′ | 16 |
| for | Reverse | 5′-CCGTCCTTGGTCGTAGTGA | 17 |
| MgAGS1 | | G-3′ | |
| Primers | Forward | 5′-TGGCATACAATTTCGCAGCCG | 18 |
| for | | G-3′ | 19 |
| MgFKS1 | Reverse | 5′-TTGTTATGGCCTTTGGTGG-3′ | |
| Primers | Forward | 5′-CAACTCGATCATGAAGTGCGAT | 20 |
| for | | GT-3′ | 21 |
| actin | Reverse | 5′-GCTCTCGTCGTACTCCTGCT | |
| | | T-3′ | |

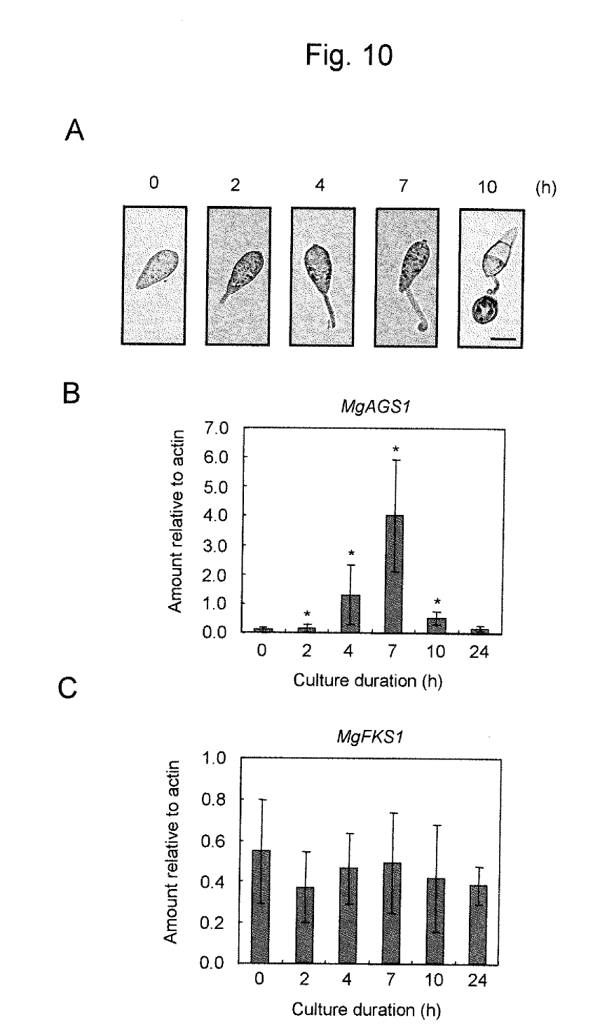

The results are shown in FIG. 10.

Spore budding was observed within 2 hours after the initiation of culture on the plastic surface and within 4 hours in the germ tube, and small early appressoriums and appressoriums were formed within approximately 7 hours and within 10 hours, respectively (FIG. 10, panel (A)). Appressoriums were thoroughly matured and melanized 24 hours after the initiation of culture (data not shown). The MgAGS1 expression level was temporarily elevated 7 to 10 hours after the initiation of culture, and, 24 hours after the initiation of culture, it was lowered to the level attained 2 hours after the initiation of culture (FIG. 10, panel (B)). In contrast, the MgFKS1 expression level was at a substantially constant level during appressorium formation (FIG. 10, panel (C)).

It was thus found that expression of the α-1,3-glucan synthase gene (MgAGS1) was specifically induced at an early stage of appressorium formation.

### EXAMPLE 6

### Expression of Cell Wall Polysaccharide Synthase Genes During Infection in Rice Cells

The transcription levels of the α-1,3-glucan synthase gene (MgAGS1) and the β-1,3-glucan synthase gene (MgFKS1) during development of infectivity in plant bodies were inspected via qRT-PCR analysis in the same manner as described above.

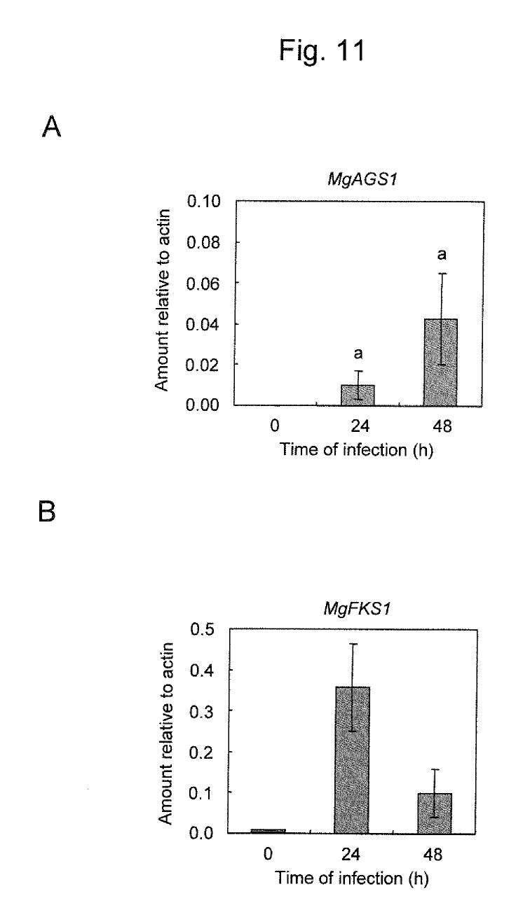

Total RNA used for qRT-PCR analysis was extracted from rice sheath cells 24 or 48 hours after inoculation with fungal rice blast pathogenic spores. The infectious hyphae developed in the rice sheath cells 24 hours after inoculation and significantly grew 48 hours after inoculation (data not shown). The MgAGS1 expression level 48 hours after inoculation was significantly higher than that 24 hours after inoculation. This indicates that MgAGS1 expression was elevated while developing infectivity (FIG. 11, panel (A)). In contrast, the MgFKS1 transcription level 48 hours after inoculation was significantly lower than that 24 hours after inoculation. This indicates that MgFKS1 expression was rapidly lowered with the elapse of time (FIG. 11, panel (B)).

Transcription of such fungal genes was not detected in total RNA extracted from the uninoculated rice sheath cells (FIG. 11, 0 hours in panels (A) and (B)).

Thus, it was found that the transcription level of the α-1,3-glucan synthase gene increased while the transcription level of the β-1,3-glucan synthase gene decreased, as infection of rice advanced.

### EXAMPLE 7

### Preparation of α-1,3-Glucanase Expression Vector

A. Construction of Plasmid (pBI333-EN4-AGL)

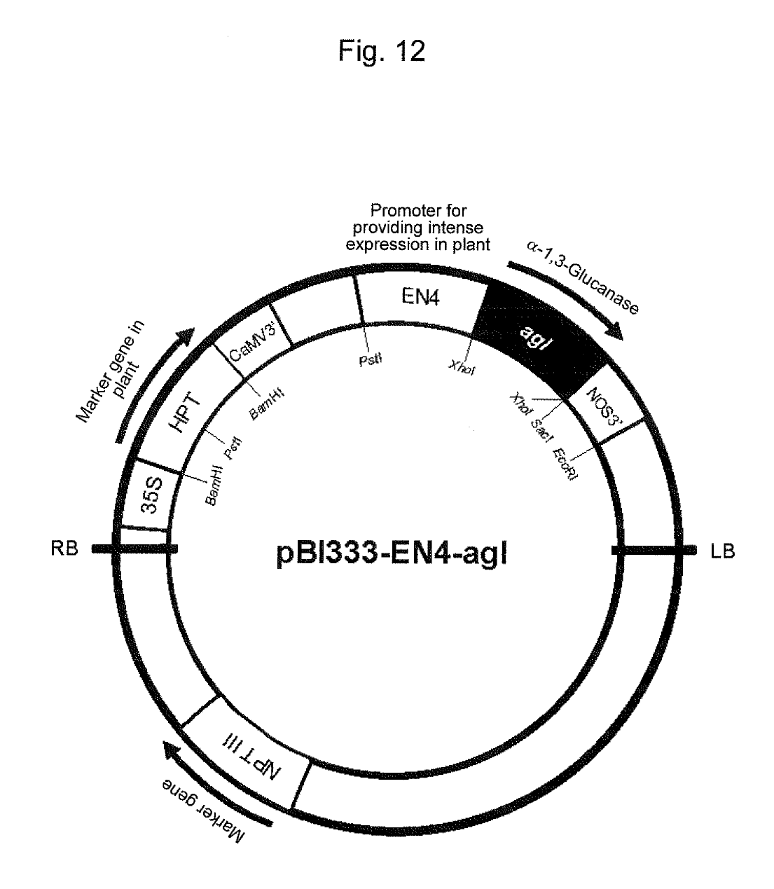

The plasmid (pBI333-EN4-AGL) having the structure shown in FIG. 12 was constructed.

pBI333-EN4-RCC2 used (Nishizawa et al., Theor. Appl. Genet., 99, 383-390, 1999) comprises the binary vector pBI121 (Clontech) and the CaMV 355 promoter:hygromycin phosphotransferase (HPT)::CaMV terminator as the selection marker cassette in the T-DNA region of the above binary vector, an artificial promoter EN4 comprising 4 repeats of an enhancer region of the cauliflower mosaic virus (CaMV) 35S promoter (provided by Dr. Hirohiko Hirochika, the National Institute of Agrobiological Sciences; SEQ ID NO: 22), downstream thereof, RCC2 (the rice chitinase gene Cht-2; Accession Number X56787), and the nopaline synthase gene terminator (NOS3′). pBI333-EN4-RCC2 was cleaved with SpeI and SacI to remove RCC2, and the SpeI-SacI fragment of AGL (SEQ ID NO: 23) encoding α-1,3-glucanase, which had been cloned in advance, was ligated thereto to complete the construction of pBI333-EN4-AGL.

B. Construction of Plasmid (pBI333-EN4-RCC2SS/AGL)

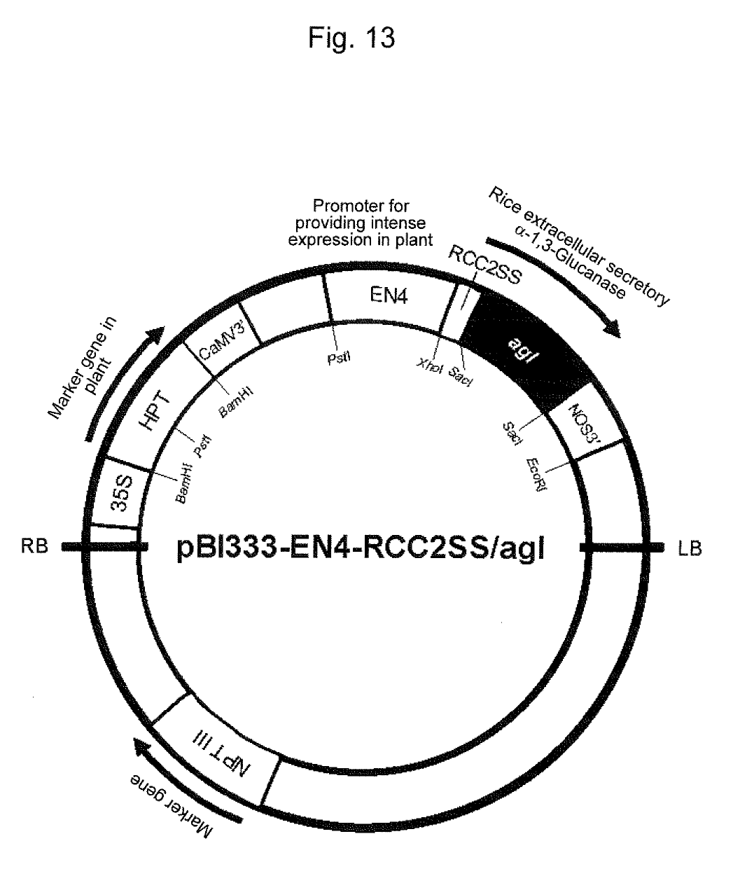

The XhoI-SacI fragment of the extracellular secretion signal (RCC2SS) was ligated to pBI333-EN4-AGL prepared above to construct the plasmid (pBI333-EN4-RCC2SS/AGL) shown in FIG. 13.

C. Construction of Plasmid (pTN2/E12Ω-RCC2SS/AGL)

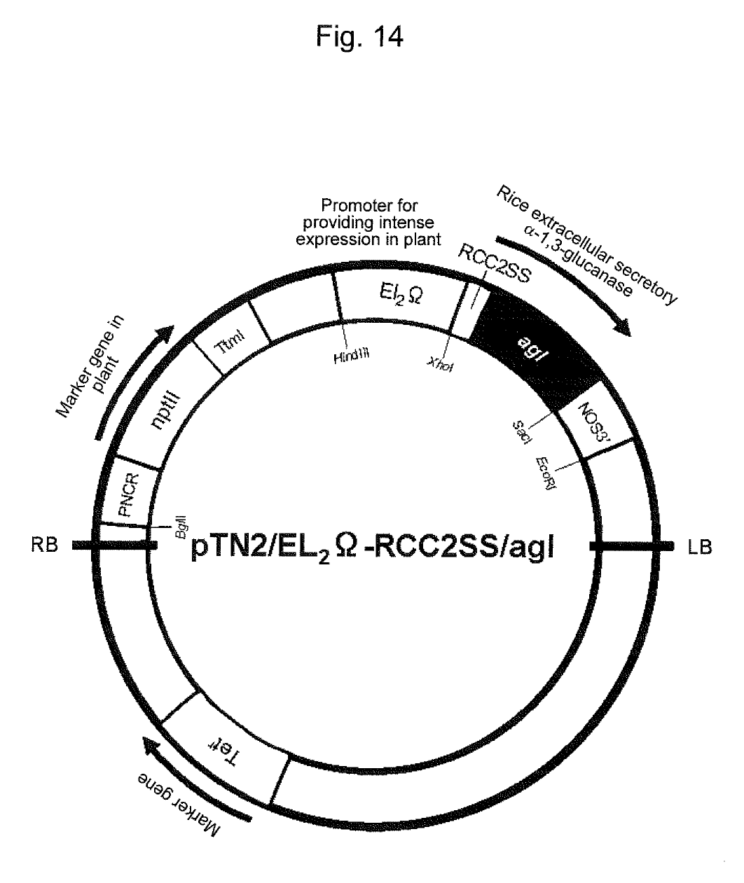

A E12Ω promoter which is a promoter for providing intense expression in plants (see Plant Cell Physiol., 40 (8): 808-817, 1999 regarding the Ω sequence and Plant Cell Physiol., 37 (1): 49-59, 1996 regarding the E12Ω promoter) was used to prepare the plasmid (pTN2/E12Ω-RCC2SS/AGL) comprising the AGL gene downstream of the RCC2SS sequence shown in FIG. 14.

This plasmid comprises nptII as the marker gene in plants, PNCR as the promoter for expressing nptII, and the Ttml sequence as the terminator (Fukuoka, H. et al., 2000, Plant Cell Rep., 19: 815-820).

D. Construction of Plasmid (pMLH7133-Sp/AGL)

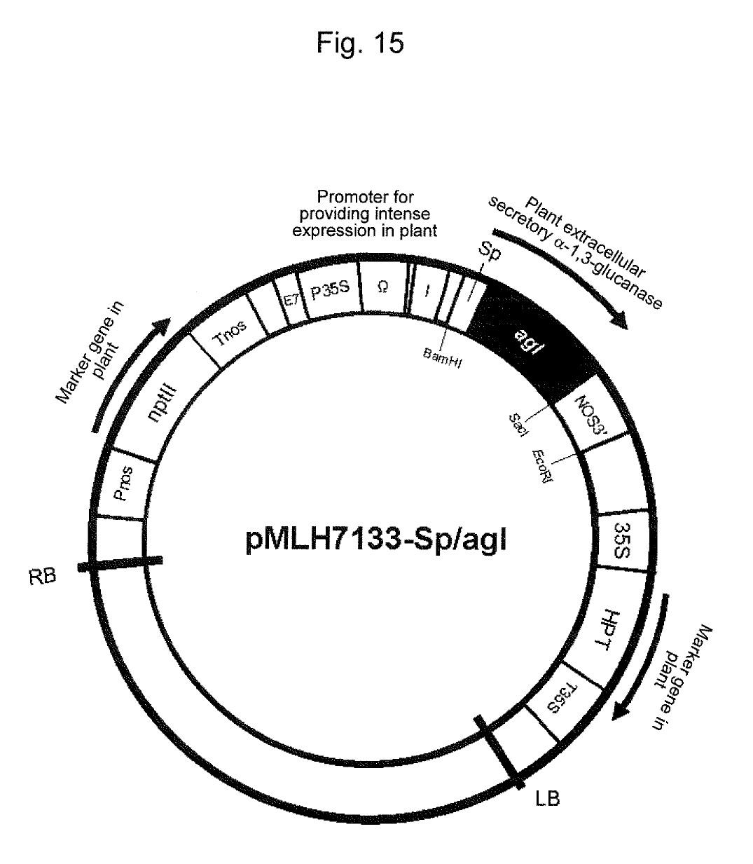

pMLH7133 used (Mochizuki et al., Entomologia Experimentalis et Applicata 93: 173-178, 1999) comprises the binary vector pBI121 (Clontech) and the nopaline synthase gene promoter (Pnos)::kanamycin phosphotransferase gene (nptII)::nopaline synthase gene terminator (Tnos) and the cauliflower mosaic virus (CaMV) 35S promoter (P35S)::hygromycin phosphotransferase gene (HPT)::CaMV 35S terminator (T35S) as the selection marker cassettes in the T-DNA region of the above binary vector, and a promoter for intense expression in plants comprising an intron sequence (see E7::P35S::Ω::I, Plant Cell Physiol., 37 (1):49-59, 1996). In a region downstream of the promoter for intense expression in plants comprising the intron sequence, the plasmid (pMLH7133-Sp/AGL) shown in FIG. 15 having the transcription initiation region and the secretory signal peptide region (Sp sequence; SEQ ID NO: 24) of the tobacco (Nicotiana tabacum) PR1a gene and the AGL gene was prepared.

### EXAMPLE 8

### Preparation of Transgenic Plant

(1) Introduction of AGL Gene into Agrobacterium

In accordance with the method of Nagel et al. (Microbial. Lett., 67, 325, 1990), the pBI333-EN4-AGL binary vector into which the α-1,3-glucanase (AGL) gene had been introduced is introduced into Agrobacterium tumefaciens (the EHA105 or LBA4404 strain) via electroporation. Thereafter, culture is conducted in LB medium(0.5% NaCl, 1% Bacto trypton, 1% Yeast extract) containing 50 μg/ml kanamycin or 50 μg/ml hygromycin at 28° C. for 2 days to obtain transformed Agrobacterium.

### (2) Introduction of AGL Gene

(2-1: Gene Introduction into Rice)

Rice plants were transformed via ultra-rapid transformation (JP Patent No. 3,141,084 or Toki et al., Plant Journal, 47, 969-976, 2006). Agrobacterium was sterilized with the use of Meropen (Dainippon Sumitomo Pharma Co., Ltd.). Specifically, gene introduction was carried out in the following manner.

A suspension of the transformed Agrobacterium prepared in (1) above and seeds of precultured rice plants via ultra-rapid transformation (Oryza sativa variety: Nipponbare) were co-cultured in 2N6-AS medium (30 g/l sucrose, 10 g/l glucose, 0.3 g/l casamino acid, 2 mg/l 2,4-D, 10 mg/l acetosyringone, and 4 g/l gelrite, pH 5.2) at 28° C. in dark for 3 days. Thereafter, Agrobacterium was washed away from the seeds with the use of sterile water containing 25 mg/l Meropen, the seeds were sowed in N6 medium containing 12.5 mg/l Meropen, 50 mg/l hygromycin as a selection marker, and 4 g/l gelrite (i.e., a selection medium), culture was conducted at 28° C. in dark for approximately 10 days to amplify hygromycin-resistant cells, and calluses were obtained.

The selected hygromycin-resistant calluses were transferred into a redifferentiation medium (MS inorganic salts and MS vitamins (Physiol. Plant, 15, 473-497, 1962), 6.25 mg/l Meropen, 50 mg/l hygromycin, 30 g/l sucrose, 30 g/l sorbitol, 2 g/l casamino acid, 2 mg/l kinetin, 0.002 mg/l NAA (naphthalenacetic acid), and 4 g/l gelrite, pH 5.8), and culture was continued at 28° C. in dark until calluses were redifferentiated.

The redifferentiated calluses were sowed in a rooting medium (i.e., hormone-free MS medium supplemented with 6.25 mg/l Meropen and 25 mg/l hygromycin (a composition of MS medium: 6.25 mg/l Meropen, 50 mg/l hygromycin, 30 g/l sucrose, 30 g/l sorbitol, 2 g/l casamino acid, and 4 g/l gelrite, pH 5.8). The resultants were transferred to a fresh rooting medium approximately 10 days later. Approximately 1 week thereafter, the transformed plants were subjected to naturalization for 2 to 3 days when they were grown and transferred to a pot filled with Kureha baiyoudo-D (Kureha culture-soil-D, tradename, Kureha Corporation), and the plants were allowed to grow in a greenhouse.

(2-2: Confirmation of Incorporation into Genomic DNA)

Among the rice plants into which the AGL gene had been introduced, genuine recombinant rice plants into which the AGL gene had been incorporated in the genomic DNA were inspected via PCR.

Genomic DNA was isolated from rice leaves with the use of the QIAGEN DNeasy mini kit (tradename, QIAGEN). The isolated DNA was used as template DNA to amplify a partial sequence of the AGL gene and that of the constitutively expressed OsUbq1 gene as the internal control via PCR, and amplified fragments were confirmed via electrophoresis. Gene-specific primers were designed in order to amplify a unique sequence (approximately 300 bp) of the relevant gene (SEQ ID NOs: 25/26: forward/reverse primers for AGL; and SEQ ID NOs: 27/28: forward/reverse primers for OsUbq1).

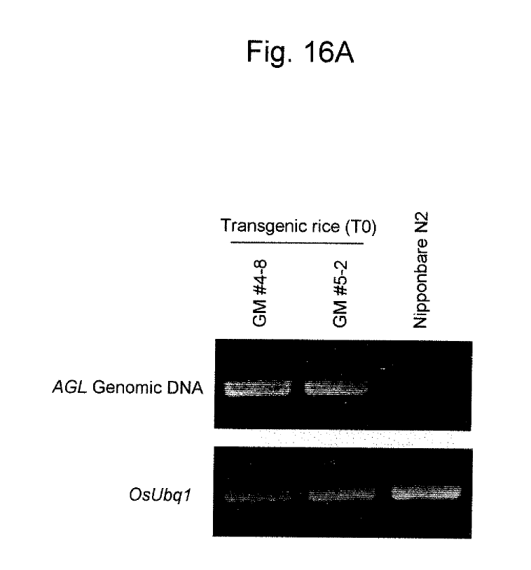

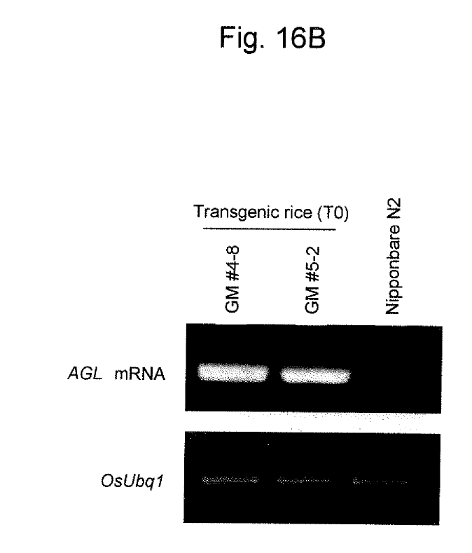

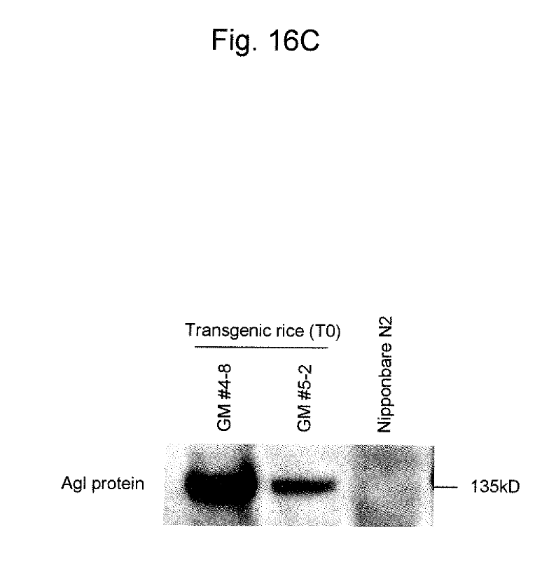

The results are shown in FIG. 16A. GM #4-8 and GM #5-2 are transgenic rice plants of the T0 generation, and Nipponbare N2 is a non-recombinant rice plant. The upper panel shows the results of observation of AGL gene-specific amplified bands, and such bands are observed in the recombinant rice plants (i.e., GM #4-8 and GM #5-2). The lower panel shows the results of observation of the amplified bands of the OsUbq1 gene, and such bands are observed in all rice plants.

Thus, incorporation of the AGL gene in the genome of the recombinant rice plants (GM #4-8 and GM #5-2) was confirmed.

(2-3: Confirmation of AGL mRNA Expression)

Expression of the AGL gene in the AGL transgenic rice plants of the T0 generation was confirmed via inverted RT-PCR.

Total RNA was extracted from leaves of the transgenic rice plants (GM #4-8 and GM #5-2) and a non-recombinant rice plant (Nipponbare N2) with the use of the QIAGEN RNeasy Plant mini kit (tradename, QIAGEN), PCR was carried out with the use of cDNA synthesized with the use of the ExScript RT reagent kit (tradename, Takara) and the oligo dT primers as a template for RT-PCR and the set of primers same as those used in 2-2 above, and the resulting amplified fragments were inspected via gel electrophoresis. The set of primers for the OsUbq1 gene as in the case of 2-2 above was used as the internal control.

The results are shown in FIG. 16B. The upper panel shows the results of observation of the amplified bands of the AGL gene, and such bands are observed only in the transgenic rice plants (GM #4-8 and GM #5-2). The lower panel shows the results of observation of the OsUbq1-gene-specific amplified bands, and such bands are observed in all rice plants.

Thus, constitutive expression of the AGL gene in the transgenic rice plants (GM #4-8 and GM #5-2) was confirmed.

### (2-4: Confirmation of Agl Protein)

Whether or not the Agl protein is contained in total protein of the AGL transgenic rice plants of the T0 generation was confirmed via Western blotting.

Total protein was extracted from rice plants, proteins were separated via SDS-PAGE, Western blotting was carried out with the use of the Agl-protein-specific rabbit anti-serum and as a primary antibody and horseradish peroxidase (HRP)-bound anti-rabbit IgG as a secondary antibody, and a luminescence substrate was added and exposed to x-ray film to confirm the presence of the Agl protein (molecular weight: approximately 135 kD). The Agl protein antiserum was purified from a rabbit immunized with the Agl protein (note: Biotools Inc., 2-15-24, Takanawa, Minato-ku, Tokyo). The Agl protein used as the antigen was expressed and purified in accordance with the method of Yano et al. (2003) (Biosci. Biotechnol. Biochem., 67, 1976-1982).

The results are shown in FIG. 16C. The Agl protein-specific bands were observed only in the transgenic rice plants (GM #4-8 and GM 45-2) (molecular weight: approximately 135 kD).

Thus, constitutive expression of the Agl protein in the transgenic rice plants (GM 44-8 and GM #5-2) was confirmed.

(3) Gene Introduction into Tobacco

Tobacco is transformed by the leaf disc method of Horsch et al. (1985) (Science, 227, 1229-1231, 1985). Agrobacterium is sterilized with the use of carbenicillin. Specifically, gene introduction is carried out in the following manner.

Leaf discs cut from tobacco leaves (Nicotiana tabacum cv. Samson NN, approximately 1-month-old) were soaked in the Agrobacterium tumefaciens LBA4404 bacterial solution having α-1,3-glucanase (i.e., the bacterial solution obtained by culturing and selecting bacteria by the method described in (1) of this example, culturing the resultant in LB liquid medium containing 50 μg/ml kanamycin or 50 μg/ml hygromycin for two days and nights, and diluting and resuspending the resultants in sterile distilled water). Culture is conducted in a shoot-inducible medium (0.1 mg/l NAA, 1 mg/l BA (benzyladenine), MS inorganic salts, and MS vitamins (described in (2-1) of this example), 30 g/l sucrose, and 8 g/l agar, pH 5.7) for 2 days. Thereafter, the culture product is transferred to a shoot-inducible medium containing 50 mg/l kanamycin and 250 mg/l carbenicillin and further cultured at 28° C. in light for 2 to 4 weeks for redifferentiation.

The redifferentiated plants are transferred to a rooting medium (MS inorganic salts and MS vitamins, 30 g/l sucrose, 50 mg/l kanamycin, 8 g/l agar, and 250 mg/l carbenicillin, pH 5.7) in the same manner as in the case of preparation of transgenic rice plants and then naturalized to obtain self-propagating seeds.

(4) Gene Introduction into Tomato

Tomato is transformed by the leaf disc method of Horsch et al. (1985) (Science, 227, 1229-1231, 1985). Agrobacterium is sterilized with the use of carbenicillin. Specifically, gene introduction is carried out in the following manner.

Tomato (Solanum lycopersicum) is aseptically sowed in a seeding medium (MS inorganic salts and MS vitamins (described in (2-1) of this example), 15 g/l sucrose, and 3 g/l gelrite, pH 5.8), and the resulting cotyledons are cut and used as leaf discs. The leaf discs are soaked in the Agrobacterium tumefaciens LBA4404 bacterial solution having α-1,3-glucanase (i.e., the bacterial solution obtained by selecting bacteria by the method described in (1) of this example, culturing the resultant in LB liquid medium containing 50 μg/ml kanamycin or 50 μg/ml hygromycin for two days and nights, and diluting and resuspending the resultants in MS medium supplemented with 100 μM acetosyringone and 10 μM mercaptoethanol) for 10 minutes, the resultant is transferred to a co-culture medium (MS inorganic salts and MS vitamins (described in (2-1) of this example), 30 g/l sucrose, 3 g/l gelrite, 1.5 mg/l zeatin, and 4 μM acetosyringone, pH 5.8), and co-culture is conducted in dark at 25° C. for 3 days.

The leaf discs after co-culture is transferred to a callus-inducible medium (MS inorganic salts and MS vitamins (described in (2-1) of this example), 30 g/l sucrose, 3 g/l gelrite, 1.5 mg/l zeatin, 100 mg/l kanamycin, and 250 mg/l carbenicillin, pH 5.8) and then cultured at 25° C. for 16 hours during daylight. When calluses are formed from leaf discs and shoots are visible from the calluses, the leaf discs are cut. To accelerate shoot growth, shoots and calluses are transferred to a shoot-inducible medium (MS inorganic salts and MS vitamins (described in (2-1) of this example), 30 WI sucrose, 3 g/l gelrite, 1.0 mg/l zeatin, 100 mg/l kanamycin, and 375 mg/l Augmentin, pH 5.8), and culture is further conducted at 25° C. for 16 hours during daylight.

When shoots grow to a length of 1 to 2 cm, the shoots are cut at the root and transferred to a rooting medium (MS inorganic salts (adjusted at a concentration 0.5 times of those described in (2-1) of this example), 15 g/l sucrose, 3 g/l gelrite, 50 mg/l kanamycin, and 250 mg/l carbenicillin, pH 5.8), and the rooted plants are selected. The selected plants are naturalized to obtain self-propagating seeds.

### EXAMPLE 9

### Confirmation of Resistance of AGL Transgenic Rice Plant to Rice Blast Fungus

### (1) Resistance to Compatible Rice Blast Fungus: 1

The leaves of the AGL transgenic rice plants prepared in Example 8 were needle-inoculated with 30 μl of a spore suspension (1×10⁶ conidiospores per ml of sterile water) of compatible (pathogenic) rice blast fungus (the Ina86-137 strain), and the inoculated leaves were incubated under continuous light conditions at 25° C. Non-recombinant rice plants (Nipponbare N2) were used as control samples. As described above, the rice blast fungus are known to form an α-1,3-glucan layer on the cell wall surface thereof when infecting host plants to avoid the immune mechanism of host plants. The inoculated leaves were observed 5 days after inoculation regarding the occurrence and degree of lesion formation.

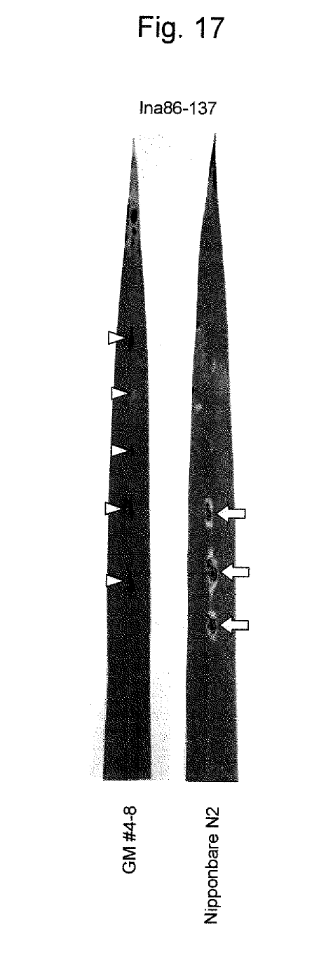

The results are shown in FIG. 17. Typical blast lesions seen at an early stage of fungal invasion were observed in leaves of the non-recombinant rice plants (Nipponbare N2) (indicated by a white arrow). However, brown spots resembling resistance reactions to fungal rice blast pathogen were observed in leaves of the transgenic rice plant (GM #4-8) prepared from Nipponbare N2 in Example 8 (indicated by a white arrow head).

The results demonstrate that the AGL transgenic rice plant of the T0 generation has resistance to rice blast fungus resulting from the expression of the foreign AGL gene.

### (2) Resistivity to Incompatible Rice Blast Fungus: 2

The leaves of the AGL transgenic rice plants (GM #4-8) prepared in Example 8 were needle-inoculated with 30 μl of a spore suspension (1×10⁶ conidiospores per ml of sterile water) of incompatible rice blast fungus (the Kyu89-246 strain), and the inoculated leaves were incubated under continuous light conditions at 25° C. Non-recombinant rice plants (Nipponbare N2) were used as control samples. The incompatible rice blast fungus (the Kyu89-246 strain) are known to be non-infectious to Nipponbare N2.

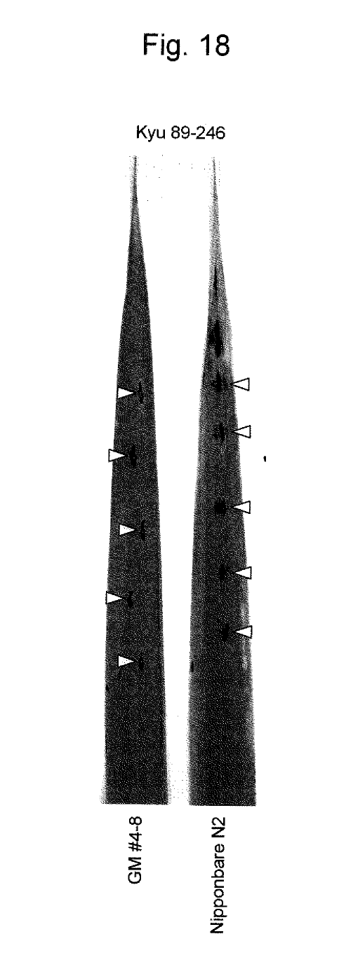

The results are shown in FIG. 18. Incompatibility of the Kyu89-246 was confirmed in Nipponbare N2 (indicated by a white arrow head). Also, incompatibility was maintained in GM #4-8 prepared from Nipponbare N2 (indicated by a white arrow head).

### EXAMPLE 10

### Confirmation of Resistivity of the AGL Transgenic Rice Plant to Cochliobolus Miyabeanus

The leaves of the AGL transgenic rice plants (GM 44-8) prepared in Example 8 were needle-inoculated with 30 μl of a spore suspension (1×10⁶ conidiospores per ml of sterile water) of wild-type strains of Cochliobolus miyabeanus (anamorph, Bipolaris oryzae) MAFF305425, and the inoculated leaves were incubated under continuous light conditions at 25° C. While Cochliobolus miyabeanus contains α-1,3-glucan as a cell-wall constitutive component, it does not form an α-1,3-glucan layer on the hyphae surface when infecting host plants, unlike rice blast fungus. Non-recombinant rice plants (Nipponbare N2) were used as control samples. The inoculated leaves were observed 5 days after inoculation regarding the occurrence and degree of lesion formation.

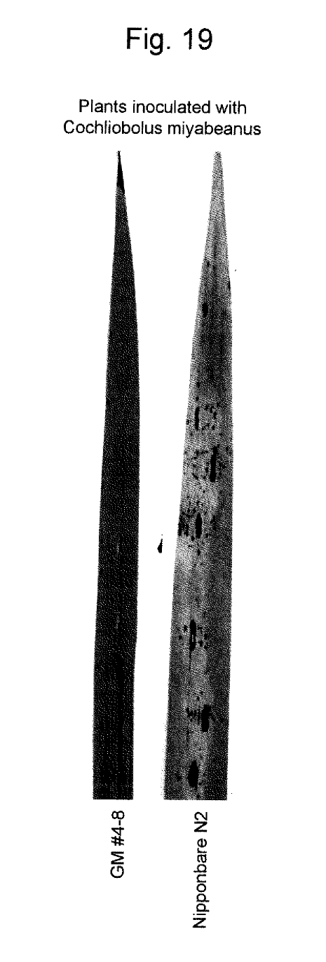

The results are shown in FIG. 19. Typical Cochliobolus miyabeanus lesions were observed in leaves of the non-recombinant rice plants (Nipponbare N2). Brown spots resembling resistance reactions were observed in leaves of GM #4-8.

The results demonstrate that the AGL transgenic rice plant of the T0 generation has resistance to Cochliobolus miyabeanus.

### EXAMPLE 11

### Confirmation of Transgenic Rice Plant of T1 Generation

### (1) Preparation of T1 Generation

The transgenic rice plants of the T0 generation were allowed to grow in a greenhouse to obtain self-propagating seeds of the next generation (referred to as the T1 or R1 generation). The seeds were sowed in a hormone-free 1/4 MS medium (1/4-fold diluted MS inorganic salts, 100 mg/l ampicillin, 50 to 100 mg/l hygromycin, and 4 g/l gellan gum). The seeds were incubated at 28° C. in dark for 1 to 2 days and then cultured under continuous light conditions for approximately 10 days. The germinated hygromycin-resistant transgenic rice plants were transferred to a pot filled with Bonsol No. 1 soil (tradename, Sumitomo Chemical Co.), and the plants were allowed to grow in a greenhouse.

### (2) Confirmation of AGL Gene Expression in T1 Generation

Whether or not the rice plants of the T1 generation obtained from the T0 transgenic rice plants into which the AGL gene had been introduced express the AGL gene was confirmed via RT-PCR. Non-recombinant rice plants (Nipponbare N2) used for the preparation of the T0 transgenic rice plants were used as control samples. The specific procedures were in accordance with the method described in Example 8 (2-3).

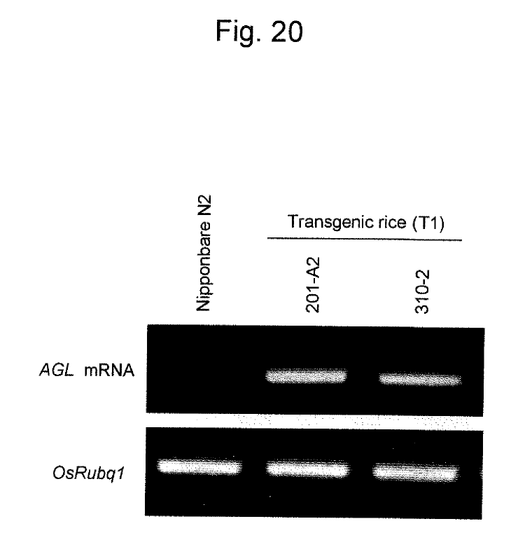

The results are shown in FIG. 20. The T1 lines (#201-A2 and 0#310-2) are both transgenic rice plants of the T1 generation obtained from self-propagating seeds of the T0 transgenic rice plants in which AGL gene had been confirmed. While the AGL amplified bands were observed in #201-A2 and #310-2, such bands were not observed in the control non-recombinant rice plants (Nipponbare N2). The OsUbq1 gene were observed in all rice plants.

Thus, constitutive expression of the AGL gene was confirmed in the T1 transgenic rice plant T1 lines (#201-A2 and #310-2).

### (3) Confirmation of Resistivity to Rice Blast Fungus in T1 Transgenic Rice Plant

The T1 transgenic rice plants (#201-A2 and 0#310-2) were spray-inoculated with 10 ml of a spore suspension (1×10⁶ conidiospores per ml of sterile water) of compatible rice blast fungus (the Ina86-137 strain), and the reaction of the rice plants was observed 5 days after inoculation. A specific procedure was in accordance with Example 9 (1).

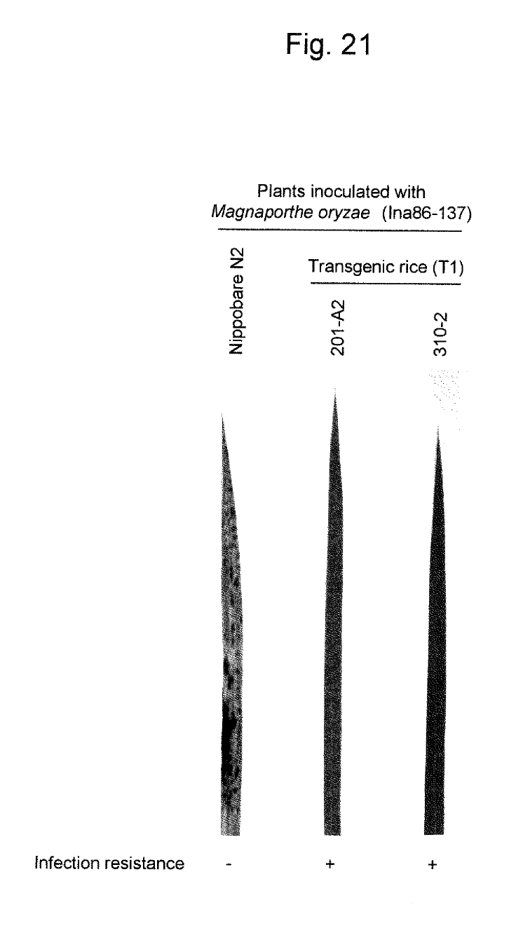

The results are shown in FIG. 21. As with the case of the T0 generation of Example 9, brown spots resembling resistance reactions to rice blast fungus were observed in the leaves of the T1 transgenic rice plants (#201-A2 and #310-2).

Thus, the AGL transgenic rice plants of the T1 generation were found to maintain resistance to rice blast fungus.

### (4) Confirmation of Resistivity to Cochliobolus Miyabeanus in T1 Transgenic Rice Plant

The T1 transgenic rice plants were spray-inoculated with 10 ml of a spore suspension (1×10⁶ conidiospores per ml of sterile water) of Cochliobolus miyabeanus, and reactions of the rice plants were observed. A specific procedure was in accordance with Example 9 (2).

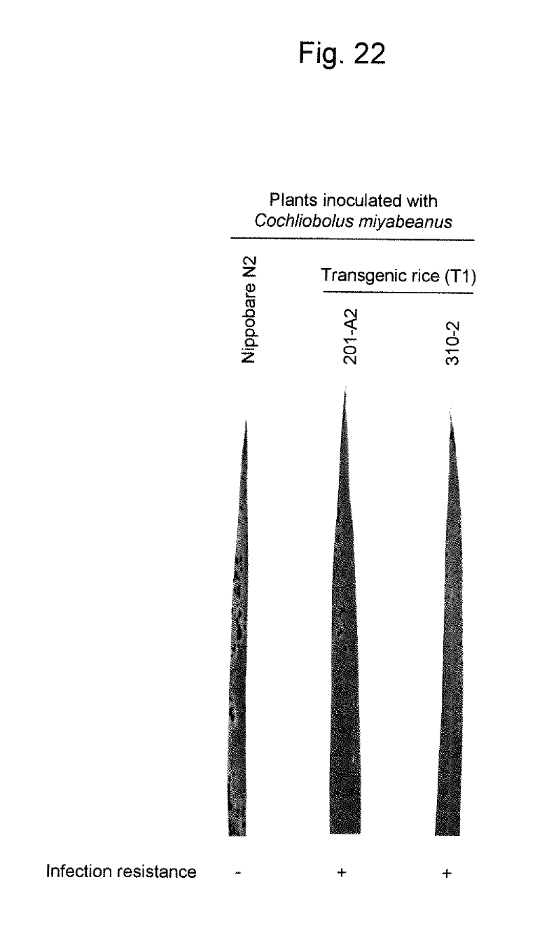

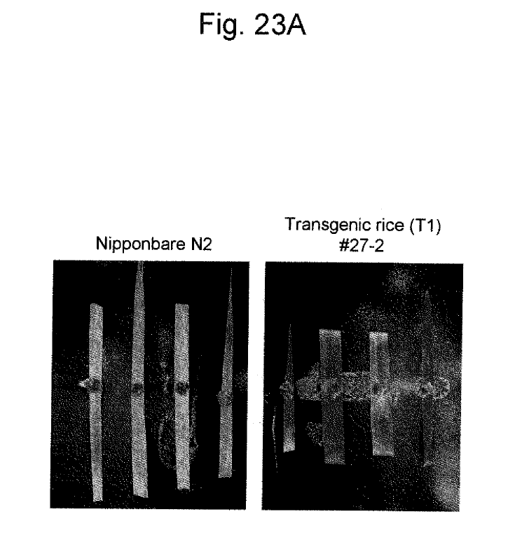

The results are shown in FIG. 22. Brown spots resembling resistance reactions were observed in the leaves of the T1 transgenic rice plants (#201-A2 and #310-2). In contrast, typical Cochliobolus miyabeanus spots were observed in non-recombinant rice plants (Nipponbare).

Thus, the AGL transgenic rice plants of the T1 generation were found to maintain resistance to Cochliobolus miyabeanus as with the T0 generation.

### (5) Confirmation of Resistance to Thanatephorus Cucumeris in T1 Transgenic Rice Plant

Resistivity of the AGL transgenic rice plants of the T1 generation to Thanatephorus cucumeris was inspected.

### (5-1) Foliar-Inoculation

Wild-type strains of Thanatephorus cucumeris (syn. Rhizoctonia solani MAFF305219) were inoculated on leaves of the T1 transgenic rice plants (#27-2) with reference to the method of Maruthasalam et al. (2007) (Plant Cell Rep., 26, 791-804). The PDA medium (24 g/l DIFCO potato dextrose broth and 1.5 (w/v) % agar) in which Thanatephorus cucumeris had grown was bored with the use of a cork borer, the medium was allowed to stand in such a manner that the flora side was brought into contact with the leaves, and incubation was carried out under continuous light conditions at 30° C. The inoculated leaves were observed 6 days after inoculation regarding the occurrence and degree of lesion formation. Thanatephorus cucumeris also contains α-1,3-glucan as a cell-wall constitutive component as with Cochlibolus miyabeanus.

As with the case of the T1 lines (#201-A2 and #310-2), the T1 line (#27-2) exhibits AGL gene expression in the AGL transgenic rice plants of the T1 generation obtained from the T0 generation prepared from Nipponbare N2 (data not shown).

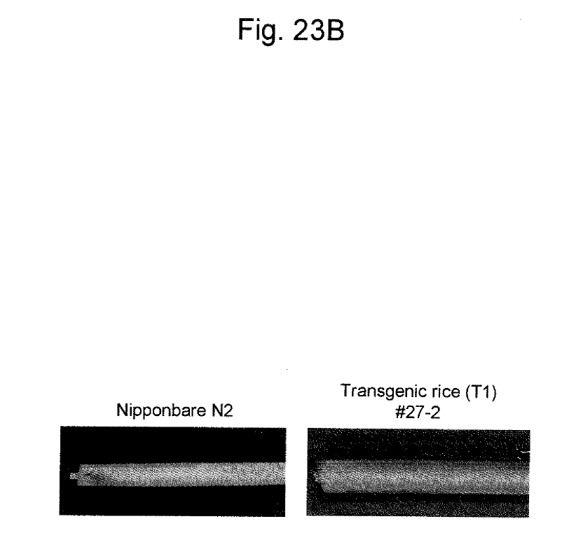

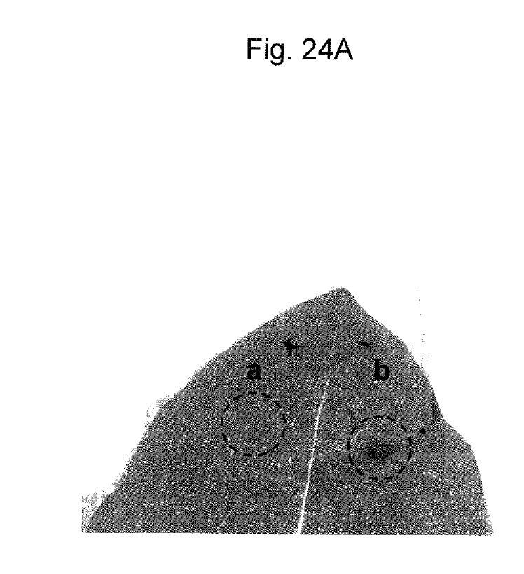

The results are shown in FIG. 23A. Leaf firing caused by Thanatephorus cucumeris was inhibited in the leaves of the T1 transgenic rice plants (#27-2). Typical leaf firing caused by Thanatephorus cucumeris occurred in Nipponbare N2.

Thus, the AGL transgenic rice plants of the T1 generation were found to have resistance to infection with Thanatephorus cucumeris.

### (5-2) Leaf Sheath Inoculation

The hyphae of wild-type Thanatephorus cucumeris strains were collected with the use of a tip of a toothpick, rubbed on the cut surfaces of the leaf sheaths of the T1 transgenic rice plants (#27-2) and Nipponbare N2, and incubated under continuous light conditions at 30° C. The inoculated leaves were observed 6 days after inoculation regarding the occurrence and degree of lesion formation.

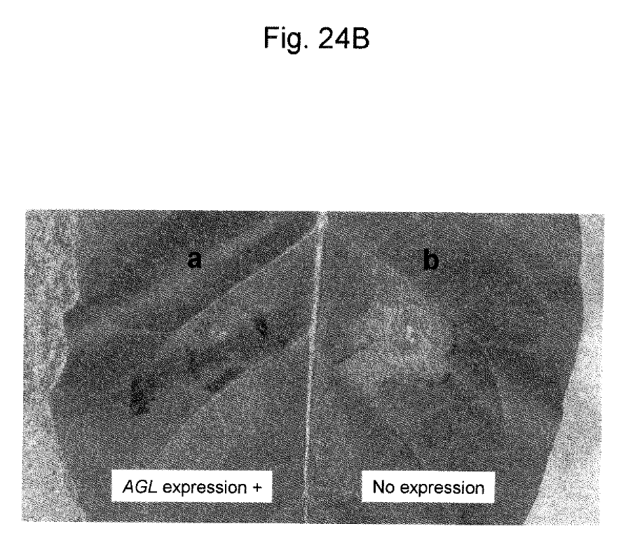

The results are shown in FIG. 24B. Firing caused by the sheath blight disease fungus was inhibited in the leaf sheath of #27-2. However, leaf sheath firing caused by the sheath blight disease fungus was serious in Nipponbare.

Thus, the AGL transgenic rice plants of the T1 generation were found to have resistance to infection with Thanatephorus cucumeris in the leaf sheath, as well as in the leaves.

### EXAMPLE 12

### Inhibition of Infection of Tobacco Leaves with Botrytis Cinerea

Botrytis cinerea contains α-1,3-glucan as a cell-wall constitutive component. Accordingly, whether or not infection of host plants with Botrytis cinerea spores, which had been treated with α-1,3-glucanase in advance, would be inhibited was examined.

(1) Inhibition of Infection of Tobacco Leaves with Botrytis Cinerea Treated with α-1,3-Glucanase

Purified α-1,3-glucanase (5 μg) was added to a spore suspension of wild-type Botrytis cinerea strains (5×10⁴ conidiospores per ml of sterile water), and leaves of the tobacco (Nicotiana tabacum) Samson NN strains were inoculated with 100 μl of the mixture. As a control, PBS buffer was applied to the same leaves instead of purified α-1,3-glucanase. Thereafter, the inoculated leaves were incubated at 25° C. and observed regarding the occurrence and degree of lesion formation 3 weeks after inoculation.

The results are shown in FIG. 24A. A region within a dashed circle indicates an inoculated region. “a” represents a leaf inoculated with Botrytis cinerea spores treated with 1,3-glucanase and “b” represents a leaf inoculated with Botrytis cinerea spores suspended selectively in 1,3-glucanase-free buffer. The results demonstrate that infectivity is significantly suppressed in Botrytis cinerea spores treated with 1,3-glucanase.

The results suggest that direct application of 1,3-glucanase on the host plant surface via coating, spraying, or other means can result in prevention of infection with plant-infecting microorganisms.

(2) Inhibition of Infection of Tobacco Leaves with Botrytis Cinerea by Transient Expression of α-1,3-Glucanase

In accordance with the method of Example 8 (1), a bacterial solution of Agrobacterium tumefaciens LBA4404 carrying the α-1,3-glucanase gene and a control bacterial solution of Agrobacterium tumefaciens LBA4404 carrying no α-1,3-glucanase gene were injected into the tobacco (Nicotiana tabacum) Samson NN strains, and the plants were incubated for 24 hours. Thereafter, 100 μl of a spore suspension (5×10⁴ conidiospores per ml of sterile water) of wild-type Botrytis cinerea strains was inoculated into the tobacco leaves that had been inoculated with Agrobacterium tumefaciens. The inoculated leaves were incubated at 25° C. and observed regarding the occurrence and degree of lesion formation 1 week later.

The results are shown in FIG. 24B. A region within a dashed line “a” indicates a region inoculated with gray mold at a site where 1,3-glucanase had been transiently expressed, and a region within a dashed line “b” indicates a region inoculated with gray mold at a site where 1,3-glucanase was not expressed.

The results demonstrate that resistance to gray mold infection is exerted when 1,3-glucanase is transiently expressed in tobacco.

### EXAMPLE 13

### Effects of Fungal Rice Blast Pathogen Control Via Inoculation with Microorganisms Secreting α-1,3-Glucanase

The efficacy of the microbial pesticide formulation of the present invention was examined with the use of the Bacillus circulans (Paenibacillus sp.) KA304 strain having the endogenous AGL gene.

### (1) Confirmation of AGL Gene Expression in the Bacillus Circulans KA304 Strain

The Bacillus circulans KA304 strains that are known to have the endogenous AGL gene (Yana et al., 2006, Biosci. Biotechnol. Biochem., 70: 1754-1763) were added to a medium for Bacillus multiplication for induction of AGL expression to which 0.5 (w/v) % α-1,3-glucan had been added as an expression inducer or non-inducible medium not supplemented with such substance (0.5 (w/v) % polypeptone, 0.5 (w/v) % yeast extract, 0.1 (w/v) % K₂HPO₄, 0.03 (w/v) % MgSO₄.7H₂O, and 0.5 (w/v) NaCl, pH 7.0), and culture was conducted overnight. As a control, the B. subtilis 168 strain free of the endogenous enzyme gene was used, After culture, total RNA was isolated from the bacteria in each of the culture solutions with the use of RNAiso (tradename, Takara). Thereafter, total RNA was treated with DNase (tradename, Nippon Gene) and cDNA was synthesized from the total RNA sample with the use of the ExScript RT reagent kit (tradename, Takara) and random hexamer primers. RT-PCR was carried out with the use of cDNA templates adjusted at the same concentration and gene-specific-primers designed to amplify an unique sequence of approximately 300-bp of the relevant gene (SEQ ID NOs: 25/26: the forward/reverse primers for AGL amplification; and SEQ ID NOs: 29/30: the forward/reverse primers for 16S rRNA amplification). PCR was carried out under the following conditions: 96° C. for 4 minutes, a cycle of 96° C. for 15 seconds, 55° C. for 30 seconds, and 72° C. for 30 seconds repeated 25 to 35 times, and 72° C. for 7 minutes in the end.

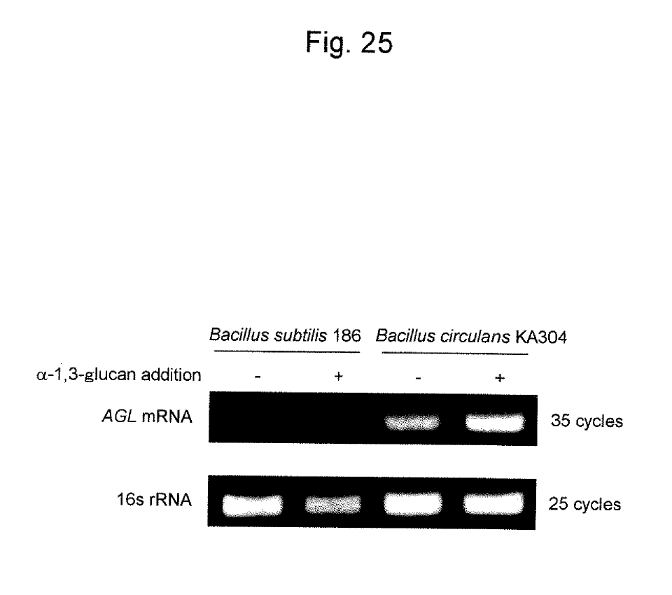

The results are shown in FIG. 25. The Bacillus subtilis 168 strain does not have the AGL gene. Accordingly, AGL gene expression is not observed under any culture conditions. In contrast, AGL gene expression was observed in the Bacillus circulans KA304 strain having the endogenous AGL gene. Further, the level of AGL gene transcription was increased in the B. circulans KA304 strain when culture was conducted with the addition of α-1,3-glucan as an expression inducer, compared with the case where culture was conducted without the addition thereof.

It was thus demonstrated that microorganisms having the AGL gene express α-1,3-glucanase at high levels by inducing expression.

(2) Resistivity of Rice Inoculated with Bacillus Subtilis to Infection with Rice Blast Fungus

The Bacillus circulans KA304 strain and the B. subtilis 168 strain were cultured by the method described in (1) of this example. After culturing, the absorbance (OD₆₀₀ nm) of each culture solution was adjusted at 0.5, and 10 ml of bacterial suspension was prepared. The resulting suspension was spray-inoculated to the cut leaves of the rice variety (LTH; rice that had developed the fourth node was used), and the inoculated leaves were incubated under continuous light conditions at 25° C. As a control, sterile water was used instead of the bacterial suspension. The cut leaves were spray-inoculated with 10 ml of a spore suspension (1×10⁶ conidiospores per ml of sterile water) of wild-type rice blast fungus (i.e., the Guy11 strains) 3 hours later, and the inoculated leaves were incubated under continuous light conditions at 25° C. Lesions that had developed on the cut leaves were observed 4 days after inoculation.

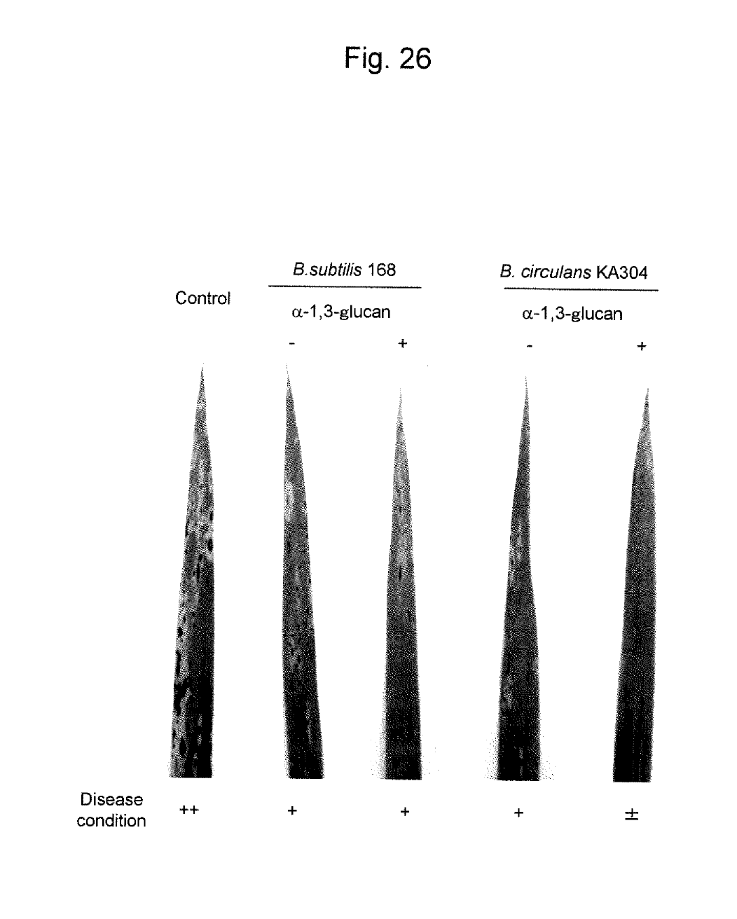

The results are shown in FIG. 26. Many developing lesions were formed in the rice plants inoculated with rice blast fungus without bacterial treatment (i.e., the control). The number of lesions slightly decreased in the rice plants inoculated with the Bacillus subtilis 168 strain that would not express α-1,3-glucanase, regardless of the addition of α-1,3-glucan at the time of culture. This is considered to be caused by other antibacterial agents secreted by Bacillus subtilis. In the case of rice plants inoculated with the Bacillus circulans KA304 strain expressing and secreting α-1,3-glucanase, the number of lesions developed was equivalent to that of the B. subtilis 168 strain when culture was conducted without the addition of α-1,3-glucan as an expression inducer. However, the number of lesions was significantly decreased when culture was conducted with the addition of α-1,3-glucan.

Thus, it was demonstrated that infection with rice blast fungus could be inhibited by spraying rice plants with B. circulans in which α-1,3-glucanase is expressed at high levels via culture with the addition of α-1,3-glucan (i.e., induction of expression). Specifically, it was verified that the microbial pesticide formulation of the present invention would be capable of effectively functioning.

### EXAMPLE 14

### α-1,3-Glucan on Cell Wall of Plant-Infecting Microorganisms that had Infected Rice

(1) Detection of α-1,3-Glucan on Cell Wall of Cochliobolus Miyabeanus that had Infected Rice

A spore suspension of Cochliobolus miyabeanus (50 μl, 1×10⁶ conidiospores per ml of sterile water) was injected into the fourth leaf sheath cells of the rice variety (Nipponbare) using a syringe, the resultant was allowed to stand at room temperature, and formation of the infectious hyphae was observed 24 hours thereafter, The leaf sheath 48 hours after inoculation was designated as a sample for α-1,3-glucan detection. In accordance with the method of Example 1, α-1,3-glucan on the Cochliobolus miyabeanus cell wall was detected.

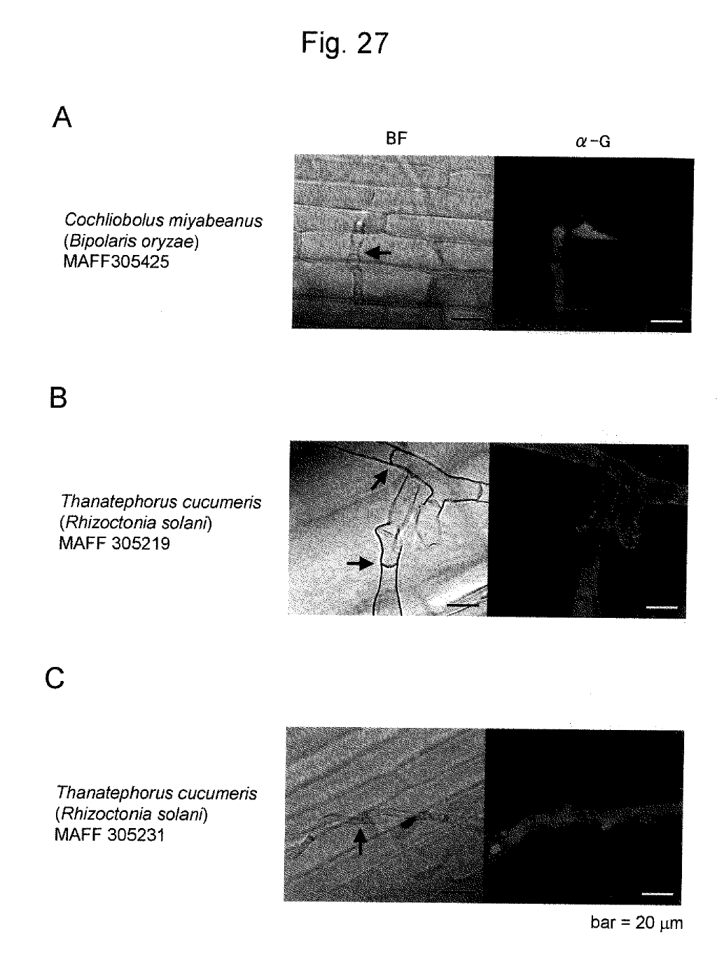

The results are shown in FIG. 27A. “BF” is an image showing inoculation in the bright field, and “α-G” is an image showing antibody detection of α-1,3-glucan with the use of a green fluorescent dye. α-1,3-glucan (α-G) was detected in the infectious hyphae of Cochliobolus miyabeanus (indicated by an arrow in the BF image).

(2) Detection of α-1,3-Glucan on Cell Wall of Thanatephorus Cucumeris that had Infected Rice

A suspension of Thanatephorus cucumeris in hyphae (50 μl) was injected into the fourth leaf sheath cells of the rice variety (Nipponbare) using a syringe, and the resultant was allowed to stand at room temperature. The leaf sheath 48 hours after inoculation was designated as a sample for α-1,3-glucan detection. In accordance with the method of Example 1, α-1,3-glucan on the Thanatephorus cucumeris cell wall was detected.

The results are shown in FIGS. 27B and 27C. FIG. 27B is an image of leaves inoculated with the Thanatephorus cucumeris MAFF305219 strain and FIG. 27C is an image of leaves inoculated with the Thanatephorus cucumeris MAFF305231 strain. A left panel shows an bright field and a right panel shows α-G. α-1,3-glucan was detected in the hyphae of Thanatephorus cucumeris.

### EXAMPLE 15

### Detection of α-1,3-Glucan on Cell Wall of Various Plant-Infecting Microorganisms

The presence of α-1,3-glucan as a constituent on the cell walls of the plant-infecting microorganisms listed in the foregoing description was confirmed.

Plant-infecting microorganisms were allowed to grow in a plant-wax-free PDA medium (24 g/l DIFCO potato dextrose broth and 1.5 (w/v) % agar) to the extent that the microorganisms would spread to the whole area of a petri-dish. Spores of the microorganisms that form spores and hyphae of microorganisms that do not form spores were collected, and a suspension was prepared with the use of sterile water. Since Golovinomyces cichoracearum is absolute parasite, a suspension of conidiospores and ascospores formed on tobacco leaves in water was used. Regarding Colletotrichum acutatum, Aspergillus niger, and Trichoderma harzianum, 1 (v/v) % potato-carrot broth (an extract obtained by boiling 20 g/l potato and 20 g/l carrots) was added to the spore suspension in order to accelerate budding or differentiation of infection structures. Thereafter, 30 μl of the suspension was added dropwise to the cover glass, incubation was carried out at room temperature overnight, and 50 μl of 3% (v/v) formaldehyde (dissolved in PBS buffer) was superposed thereon, followed by incubation at 65° C. for 30 minutes. After the resultant was thoroughly rinsed with PBS buffer, α-1,3-glucan was detected in accordance with the method of Example 1.

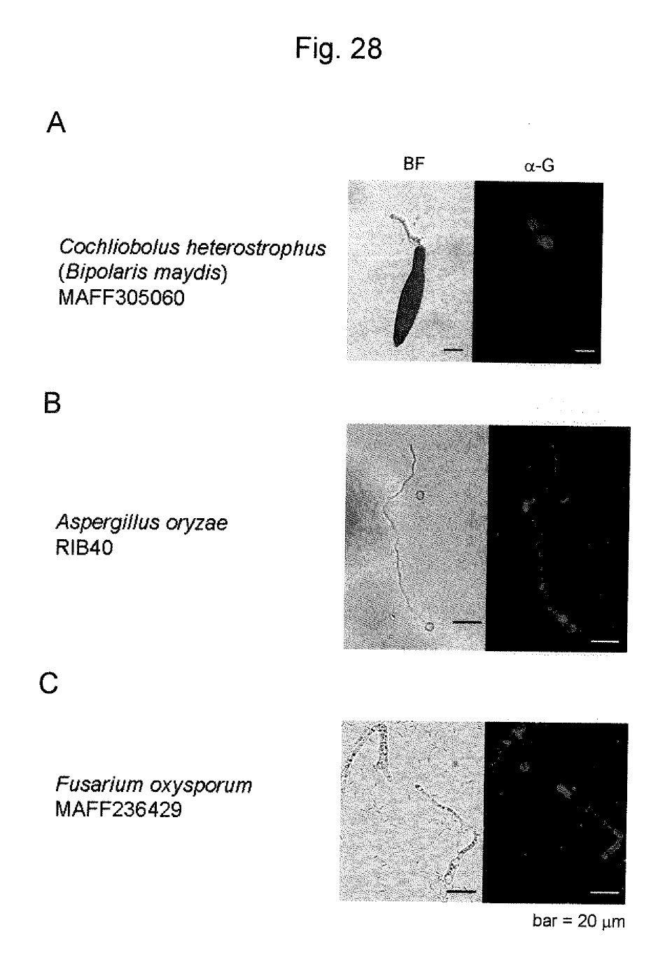

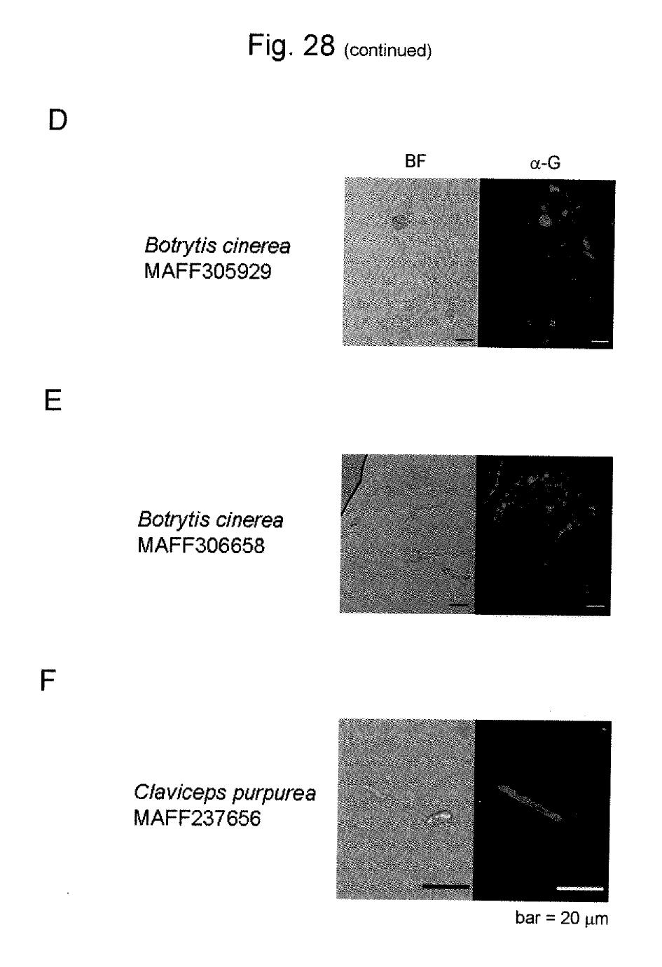

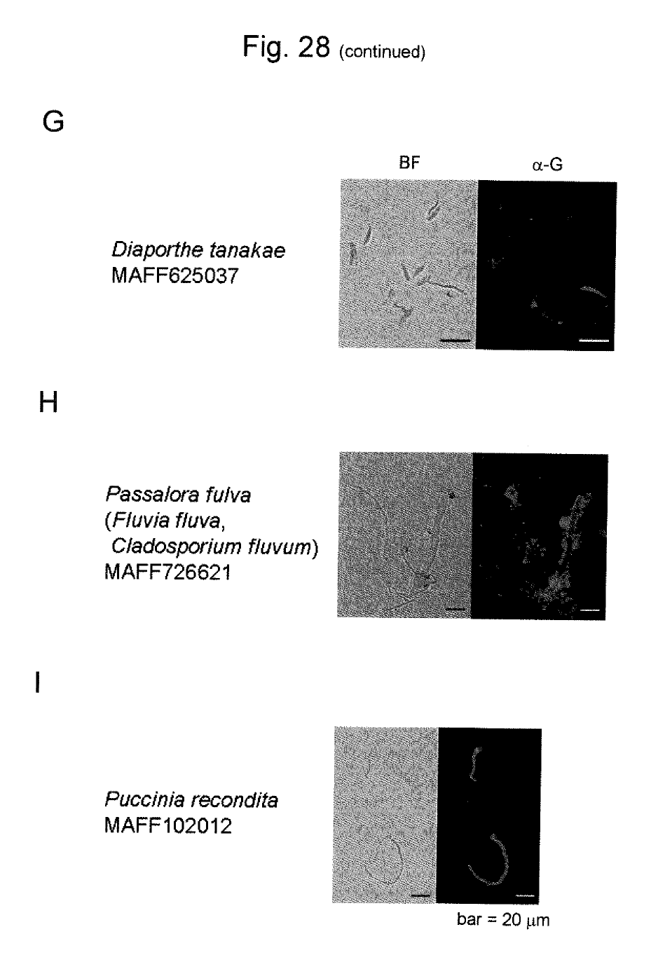

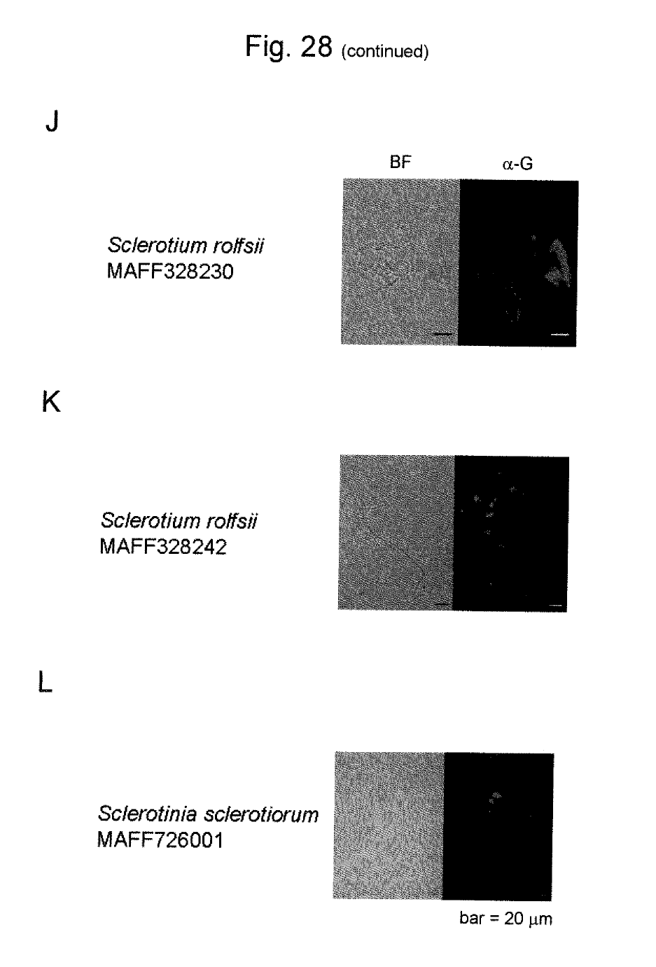

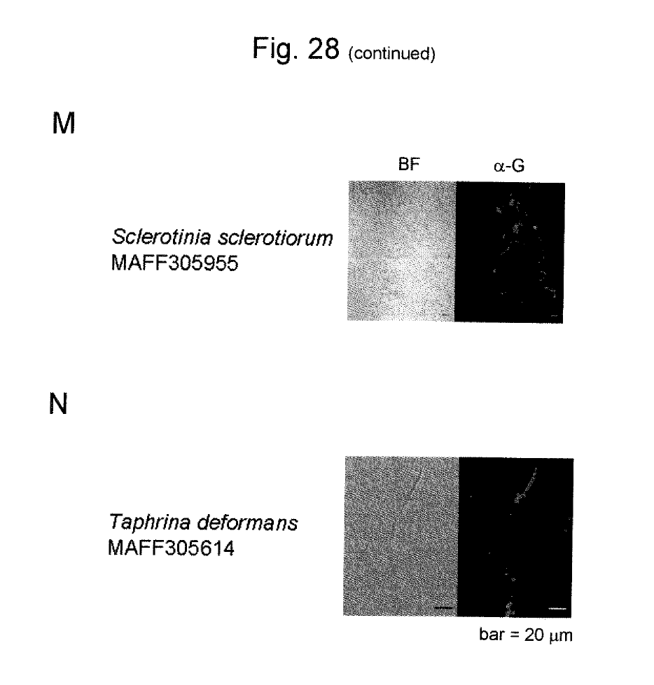

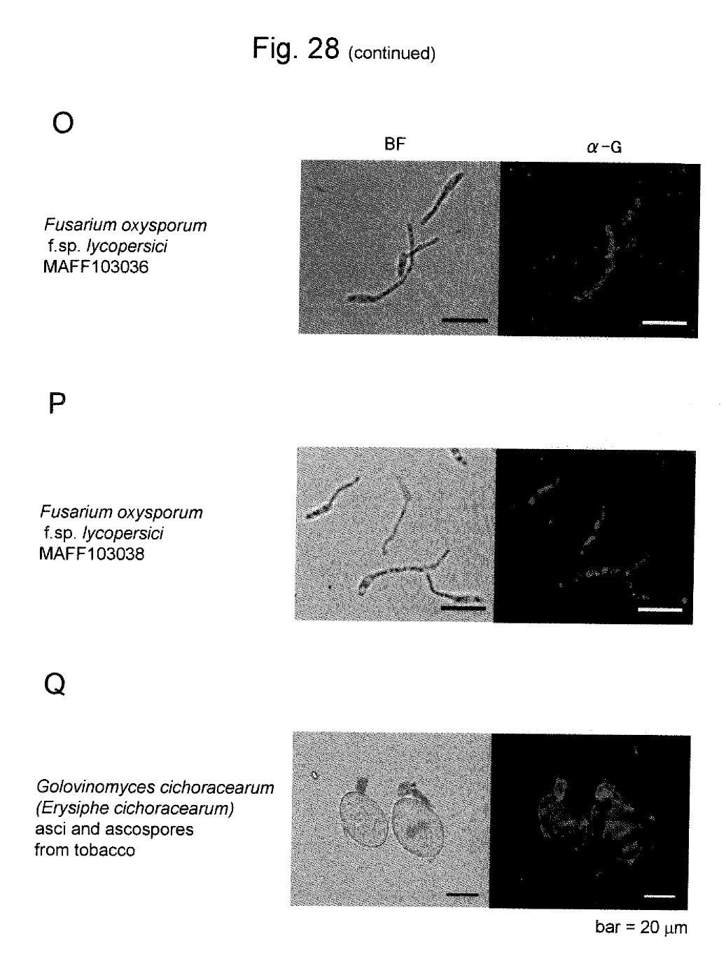

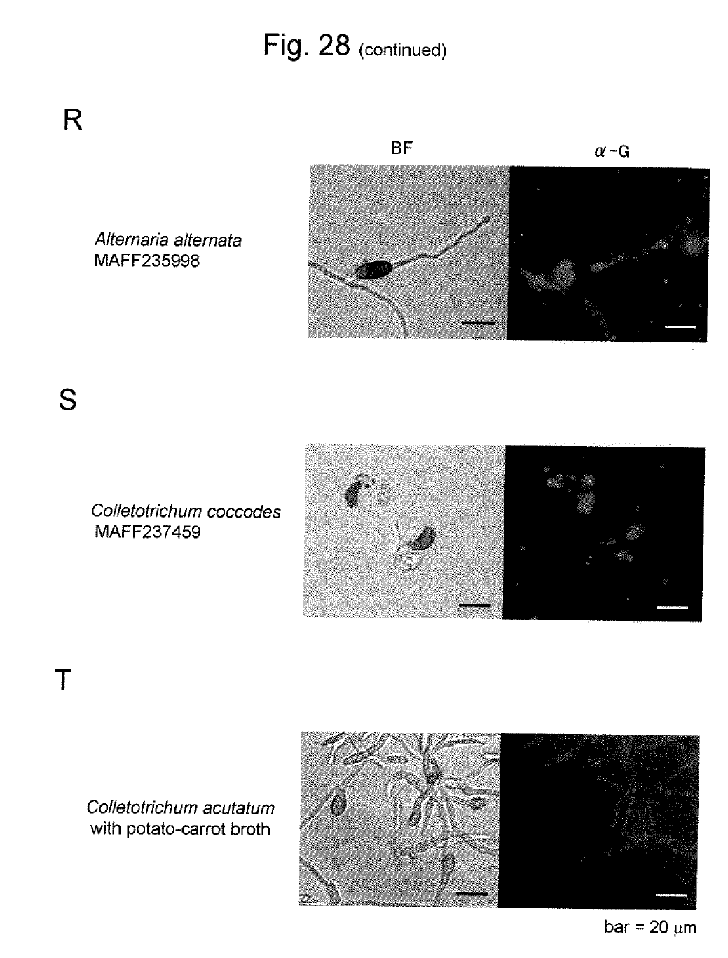

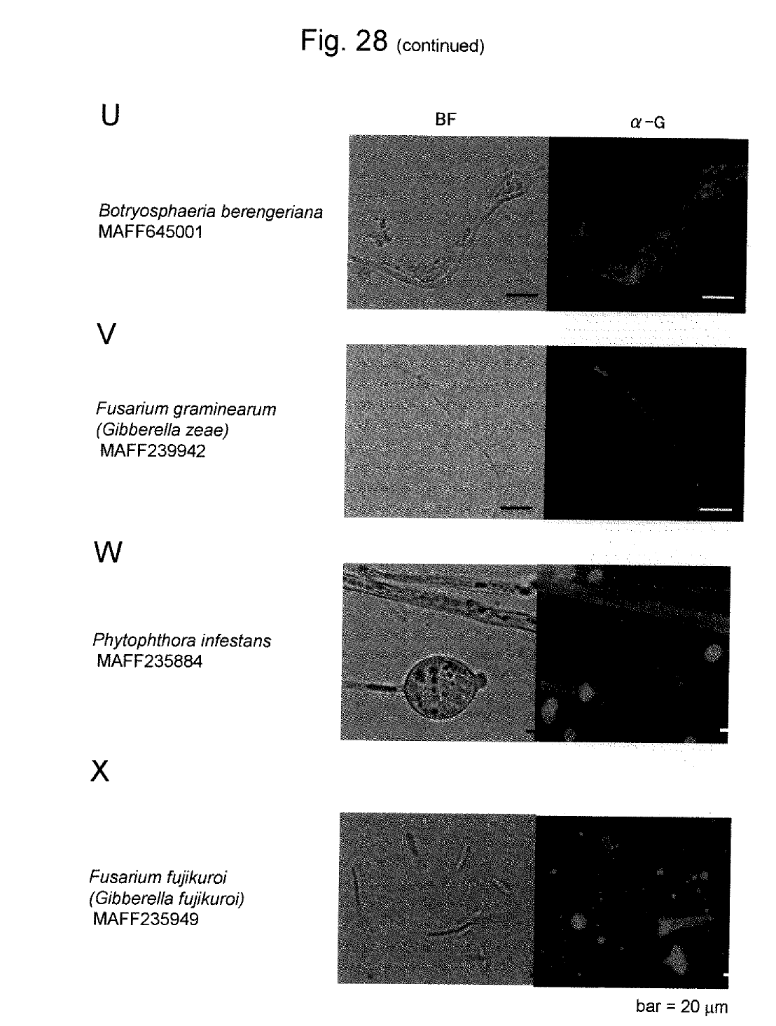

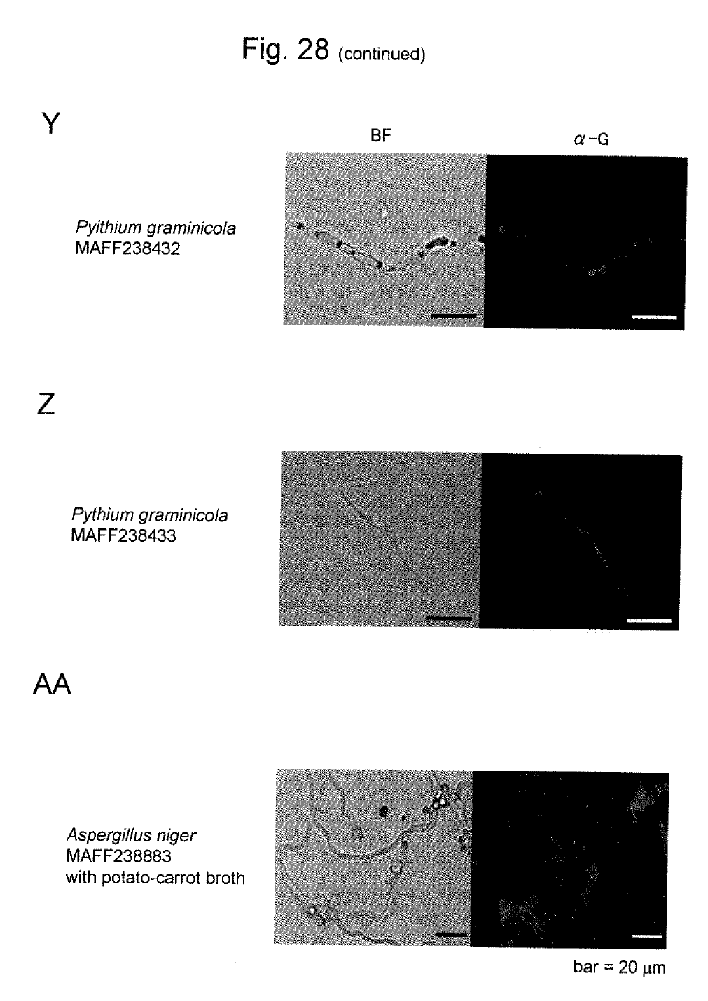

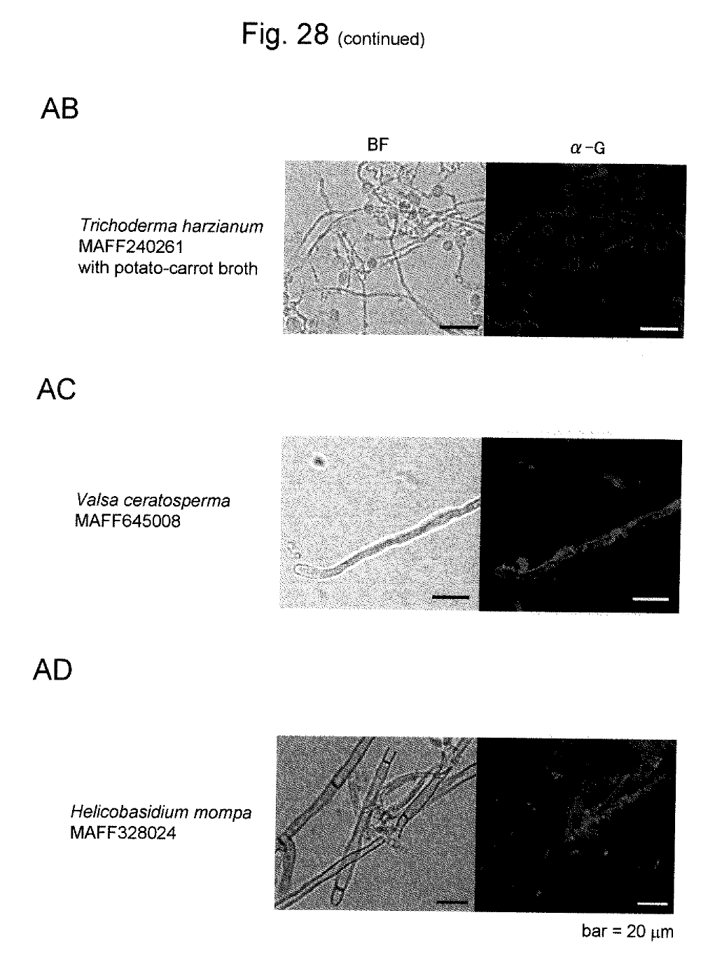

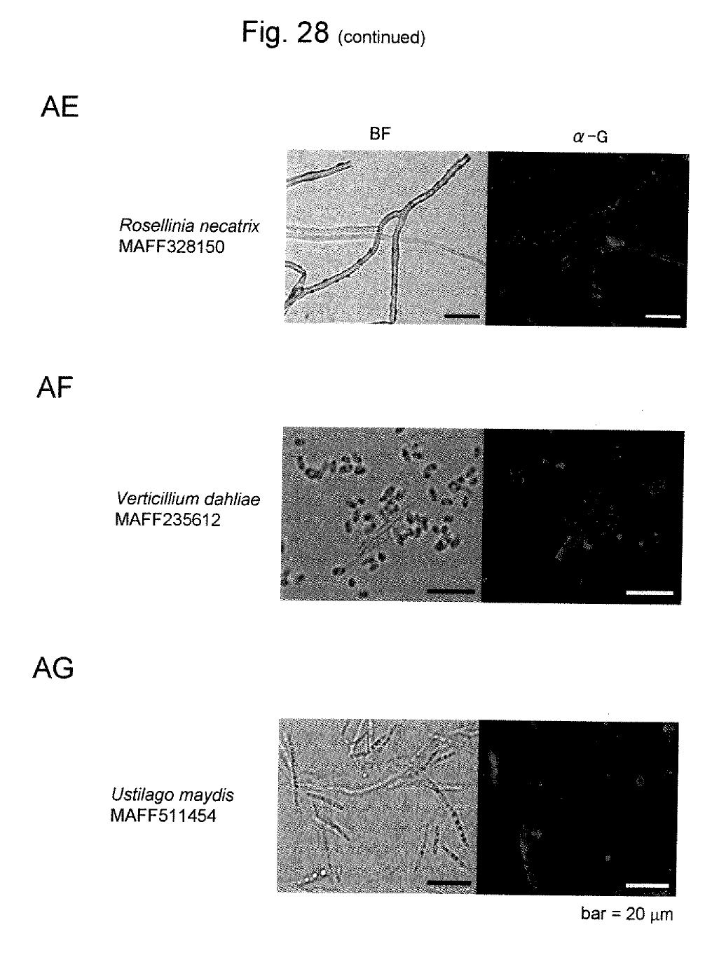

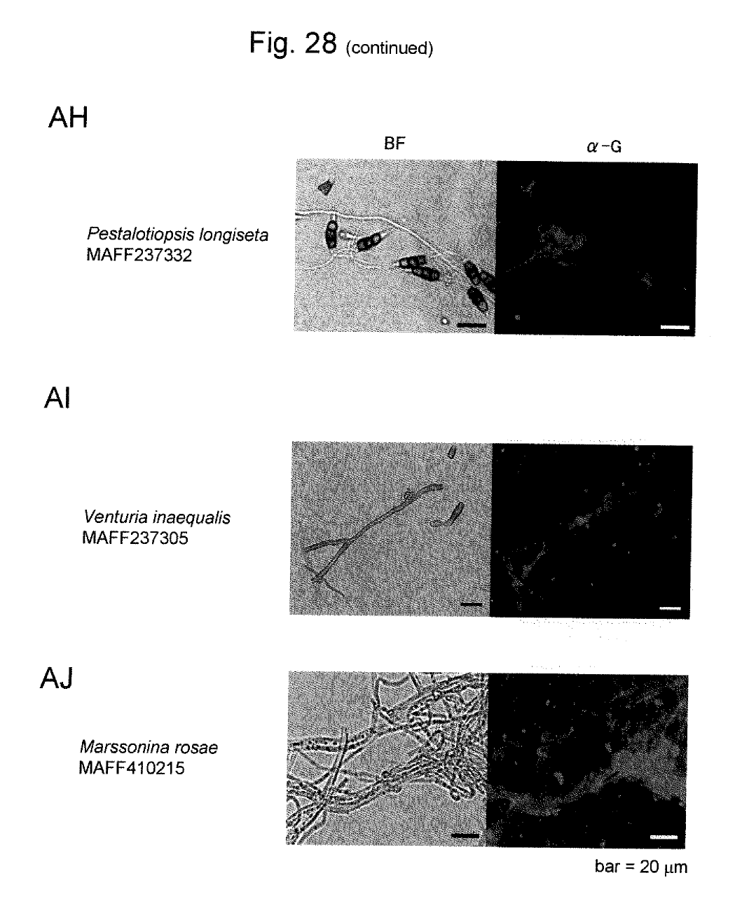

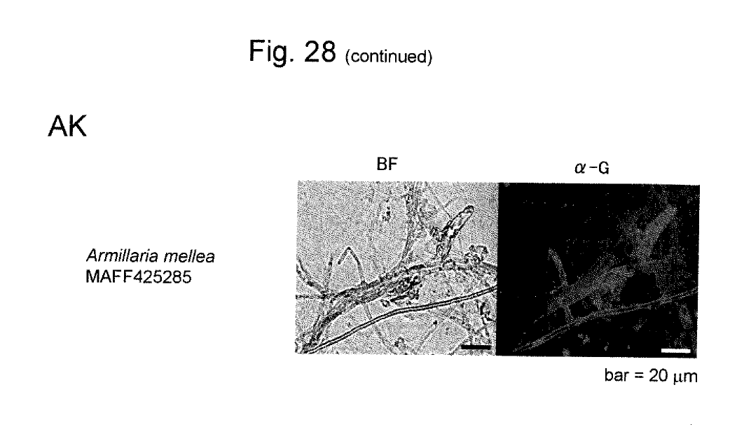

The results are shown in FIGS. 28A to 28AK. In all figures, the left panel (BF) is a photograph taken in the bright field, and the right panel (α-G) is a photograph showing antibody detection of α-1,3-glucan with a green fluorescent dye.

The names of plant-infecting microorganisms used for detection are as follows. In FIG. 28, “A” to “AK” are images showing the results of examining plants: A: the pathogens causing southern leaf blight, Cochliobolus heterostrophus (=Bipolaris maydis), MAFF305060; B: Aspergillus oryzae RIB40; C pathogens causing wilt in vegetables, Fusarium oxysporum MAFF236429; D: gray mold, Botrytis cinerea, MAFF305929; E: gray mold, Botrytis cinerea, MAFF306658; F: Claviceps purpurea, MAFF237656; G: Botryosphaeria berengeriana, Diaporthe tanakae, MAFF625037; H: pathogens causing tomato leaf mold, Passalora fulva (=Fluvia fluva or Cladosporium fluvum), MAFF726621; I: pathogens causing rust, Puccinia recondite, MAFF102012; pathogens causing southern blight, Sclerotium rolfsii, MAFF328230; K: pathogens causing southern blight, Sclerotium rolfsii, MAFF328242; L: pathogens causing Sclerotinia rot, Sclerotinia scleroiorum, MAFF726001; M: pathogens causing Sclerotinia rot, Sclerotinia sclerotiorum, MAFF305955; N: pathogens causing leaf curl, Taphrina deformans, MAFF305614; 0: pathogens causing tomato Fusarium wilt, Fusarium oxysporum f sp. lycopersici, MAFF103036; P: pathogens causing tomato Fusarium wilt, Fusarium oxysporum f sp. lycopersici, MAFF103038; Q: ascus and ascospores of pathogens causing powdery mildew of tobacco isolated from tobacco leaves, Golovinomyces cichoracearum (=Erysiphe cichoracearum); R: pathogens causing alternaria blotch on apples, Alternaria alternata, MAFF235998; S: pathogens causing tomato anthracnose, Colletotrichum coccodes, MAFF237459; T: pathogens causing strawberry anthracnose when potato-carrot broth (1 (v/v) %, an extract obtained by boiling 20 g/l potato and 20 g/l carrot) was added to a spore suspension, Colletotrichum acutatum; U: diaporthe canker apple, Botryosphaeria berengeriana, MAFF645001; V: pathogens causing red mold disease of wheat, Fusarium graminearum (=Gibberella zeae), MAFF239942; W: Phytophthora infestans, MAFF235884; X: pathogens causing rice Bakanae disease, Fusarium fujikuroi (=Gibberella fujikuroi), MAFF235949; Y: Pythium pathogens causing rice damping-off of seedlings, Pythium graminicola, MAFF238432; Z: Pythium pathogens causing rice damping-off of seedlings, Pythium graminicola, MAFF238433; AA: Aspergillus niger (black aspergillus), when adding potato-carrot broth (1 (v/v) %) of 20 g/l potato and 20 g/l carrots; AB: Trichoderma harzianum, MAFF240261, when adding potato-carrot broth (1 (v/v) %) of 20 g/l potato and 20 g/l carrots; AC: pathogens causing apple Valsa canker, Valsa ceratosperma, MAFF645008; AD: the violet root rot fungi, Helicobasidium mompa, MAFF328024; AE: the white root rot fungi, Rosellinia necatrix, MAFF328150; AF: pathogens causing Verticillium wilt, Verticillium dahliae, MAFF235612; AG: the maize smut fungi, Ustilago maydis, MAFF511454; AH: the causal fungi for tea gray blight disease, Pestalotiopsis longiseta, MAFF237332; AI: the causal fungi for apple black spot, Venturia inaequalis, MAFF237305; AJ: the causal fungi for rose black spot, Marssonina rosae (=Diplocarpon rosae), MAFF410215; and AK: the causal fungi for armillaria root rot, Armillaria mellea, MAFF425285. α-1,3-glucan was detected in every cell wall.

The above results demonstrate that α-1,3-glucan is present in many plant-infecting microorganism species as a cell-wall constitutive component. Accordingly, the transgenic plant into which the AGL gene has been introduced or the microbial pesticide formulation of the present invention is considered to be capable of preventing or inhibiting infection with microorganisms by degrading cell walls of these many plant-infecting microorganism species each comprising α-1,3-glucan on its cell wall.

### INDUSTRIAL APPLICABILITY

According to the method for preventing or inhibiting infection with plant-infecting microorganisms according to the present invention, infection of host plants with plant-infecting microorganisms containing α-1,3-glucan on the cell wall can be prevented or inhibited.

The present invention can provide a microbial pesticide formulation that is effective for prevention or inhibition of infection of plants with plant-infecting microorganisms containing α-1,3-glucan on the cell wall, regardless of specificity among plant hosts or varieties.

In addition, the method for preventing or inhibiting infection with plant-infecting microorganisms and the microbial pesticide formulation according to the present invention target cell wall components that are essential for microbial infection. Accordingly, development of resistant microorganisms is less likely to occur, advantageously.

All publications, patents, and patent applications cited herein are incorporated herein by reference in their entirety.

1. A method for preventing or inhibiting infection of a host plant with a plant-infecting microorganism comprising degrading α-1,3-glucan on the microbial cell wall with α-1,3-glucanase.

2. The method according to claim 1, wherein the plant-infecting microorganism comprises α-1,3-glucan as a constitutive cell wall component.

3. The method according to claim 1 or 2, wherein the plant-infecting microorganism forms a cell-wall-coating layer comprising α-1,3-glucan upon contact with a host plant.

4. The method according to claim 2, wherein the plant-infecting microorganism is selected from the group consisting of microorganisms of genera Botrytis, Aspergillus, Sclerotinia, Puccinia, Colletotrichum, Fusarium, Alternaria, Rhizoctonia, Sclerotium, Peronospora, Sphaerotheca, and Erysiphe.

5. The method according to claim 3, wherein the plant-infecting microorganism is of genera Magnaporthe or Colletotrichum.

6. The method according to claim 1, wherein the plant is a dicotyledonous or monocotyledonous plant.

7. The method according to claim 6, wherein the plant is a gramineous or solanaceous plant.

8. The method according to claim 1, wherein α-1,3-glucan on the cell wall of the plant-infecting microorganism is degraded by α-1,3-glucanase expressed by a foreign gene in the plant.

9. The method according to claim 1, wherein α-1,3-glucanase is brought into contact with the plant.

10. The method according to claim 1, wherein a microbial pesticide formulation comprising, as an active ingredient, a microorganism that has the α-1,3-glucanase gene and secretes α-1,3-glucanase to the outside of the cell is allowed to act on the plant.

11. The method according to claim 10, wherein the α-1,3-glucanase expression level in the microorganism is significantly higher than that of a wild type thereof at the time of normal growth.

12. The method according to claim 11, wherein the microorganism is subjected to induction of α-1,3-glucanase expression.

13. The method according to claim 12, wherein the induction of expression is addition of α-1,3-glucan.

14. The method according to claim 10, wherein the α-1,3-glucanase gene is an endogenous gene.

15. The method according to claim 14, wherein the microorganism is of genera Bacillus, Paenibacillus, Aspergillus, and/or Trichoderma.

16. A method for preparing a plant exhibiting resistance to microbial infection comprising a step of transforming a plant with an expression vector comprising a gene encoding α-1,3-glucanase.

17. An expression vector comprising a gene encoding α-1,3-glucanase used for the method according to claim 16.

18. A plant cell containing the expression vector according to claim 17.

19. Plant tissue containing the plant cell according to claim 18.

20. A plant body containing the plant cell according to claim 18 or plant tissue.

21. A seed obtained from the plant body according to claim 20.

22. A microbial pesticide formulation comprising, as an active ingredient, a microorganism that has the α-1,3-glucanase gene and secretes α-1,3-glucanase to the outside of the cell.

23. The microbial pesticide formulation according to claim 22, wherein the α-1,3-glucanase expression level in the microorganism is significantly higher than that of a wild type thereof at the time of normal growth.

24. The microbial pesticide formulation according to claim 23, wherein the microorganism is subjected to induction of α-1,3-glucanase expression.

25. The microbial pesticide formulation according to claim 24, wherein the induction of expression is addition of α-1,3-glucan.

26. The microbial pesticide formulation according to claim 22, wherein the α-1,3-glucanase gene is an endogenous gene.

27. The microbial pesticide formulation according to claim 26, wherein the microorganism is of genera Bacillus, Paenibacillus, Aspergillus, and/or Trichoderma.


SEQUENCE LISTING (33 sequences)

SEQ ID NO: 1 - DNA, 1570 bp, Magnaporthe grisea
caggtgtggttccacagacaaacggaacccctgttcaaacgccccccacggtgcatcgttagtcccaaatctggactcgtgttgttacaagcatggaattccacgagagctagccaagggatcttgtcaaaatcttactagtctagtaactgtatgccacacatcaggtacacatgtagtacacatgcgagagacaggggttgtacagtacatacagtacagtacgtgtgcaaatgcgcgggacgaatgtcccgggtaagacttttaagataacgtcgtggtatcaattgcacatagtcagtaagcgacagagaatatcccgtatgctacgctcgacactacgtcacaaagcgcctaacccaagtgccagtattgaagcacaaattcaattaaatcaaggccagcgcattcaaactttccacatagtcgtgcagtcgcatacaaatcgcaggcctcttctcttttcttcccacccccggcggagcgttgaaccaaattgggaacagccgatacgaggccagaccagaaaaagtgggtgctaaggttgttggaacatggcaaccccggtcagcagaaaaaaaaaaaaaagagaattcgctgggtcgtctcctgccctgccaatgtgaaagggcgagtcaagaccaagcaagcacgcttgggaatcaggtaccaagtcttgtatgcactactaagcaaccgcccgcctggcctgcgggatcgaaaactttttttttcctcttcttcttcagcggccccgactctttgggcgtgtgcgacactccaccgcaggcacacacgcacaaaacacacaccacccctcttctccccttgcccgtcttgccacgtctcggcgctgaggaagcacccgcccctgcgtgtcgttatcccacaccccgttactcgttaaagacaaaaaaaaaaaaaccgcctttggtcactttatggtggtatcgtaaaaaaacatggtagcggctcgcttttctgagctgggtttctgttcctctgggctcatgttcgcattatcacaccatgcccagcggttagaattggaggctggaagtcccgctccgtccgactgggccgtcttttccatactcatcacatacatacgtcagttcgatgtatcttttttttttcttttcttttctgacttcttttttcccttcccttgcctacctacaggttggagctgccgaagatcccgggccgagtgggctatcctttttttgaacccaagttgtatccataagggcctgtcaaccttgtacttgcgtagatcttgacctccttgcctcgtatcttttggctgtgagaggtagtcaaggatacccagttttgaaaatctcgataccttccttgcctgcctcctccttcctttgccccctttcccccccttttttcctctattttttttttcctactctgttcagcctcccaaaccagttccattctggtccccgcccctgccgggtcaagcccaacacgaactgtcgtttgggtgcacgtagtcgtagataattgtggtgccctggttccgggccaatccagtagtcccatatctccccagtgg

SEQ ID NO: 2 - DNA, 1382 bp, Magnaporthe grisea
ggttcatccatttgctttcgtgtccattggtttgattgcgtgcagactaccttggcttcaatgaaactcgaccgtgctgctagacctttcgtggtgtttccgttgtcgctcctcggtagttctatgtggcattctgtaattaccgggatgaccaagaaaccaggatggggatggggtttgtaagcgatctcgaatggtcaaatggccaccttcctttgtgagcgggttttcatacaggcaaatctctgggtaatagcatgcagattacaagattgcactattcccagtagttggagttgataatacttgcatccaagtccaagttccgtgtagcggttgcttatccaagctcactgaggtgcttatccaagctcactgaggtgcttatccaagctcactgaggtgcttatccaagctccttgagatgggtccctcaaatccgctggaaccgggctttacaagtccgttggaattcaacccccaggcagacccctcatcaatgtgttttggttcctggtcaaatgacacacgtccctagccttgattcttgacttgactgggtttgcagtaacgttgggctccacatctccgacccccctaaagtccggcccccttgaaaaatgtaacgacgaaataccccttttataaaccgatttaaatgtaaacctatcaacgcacaataatacaatcattgtcaggctctcccggttcgctaacctcttctccatcgttcaaagcttctaaacagtccctattattttcctgtgcttcataaacgttttttttgctgaccaaaagctcgttgggatccggaatcacccttttccttttgaccggccgtaccgcctccaattttgcttttaacaaactgattttttgctgagcttcagccaataagatatccttggtttcgaagcttttttggacttttgcaaacaaaagccgtgaagtggcgttacttttttcggaccgggaaatttcctgtagttgaagtcgaatatctcgggtcgttttaggggttttccaaataagtaaagactggtcgttaattttttgggttggattttccggtgttttatccctttggaagccgttatttttacctttttcgacgttggcgttgctattttctaataaaaaagggtttaaaagtggttttgccaagttcaccggccataaccccgtcgtacgccaaccagattgaatggtttttgctataaatgcttttgatctggcttttcgatagcaaagtagaaagtttcgtttcccaacaaccgttgaacagcaaaactggtttacaaatcccaggtgacgtcgataagcttcctttaacggcccaaaaaccgataaatccaacggttgaagaacgtgtgacgaatgag

SEQ ID NO: 3 - DNA, 18 bp, Artificial
  Forward primer for cloning of MgAGS1 upstream sequence
caggtgtggttccacaga

SEQ ID NO: 4 - DNA, 29 bp, Artificial
  Reverse primer for cloning of MgAGS1 upstream sequence
atgacagcgagccactggggagatatggg

SEQ ID NO: 5 - DNA, 29 bp, Artificial
  Forward primer for cloning of MgAGS1 downstream sequence
caggaggcctggttcatccatttgctttc

SEQ ID NO: 6 - DNA, 18 bp, Artificial
  Reverse primer for cloning of MgAGS1 downstream sequence
ctcattcgtcacacgttc

SEQ ID NO: 7 - DNA, 835 bp, Streptomyces hygroscopicus
  Bar gene
gctcgctgtcattttcgagacgccatctttggaagcggtggccgaatccgtactgcgcggactcgacgacgcgtaaaacgatcgaccacgtacacgagtccggacacggggcgaggaggcccggttccggcaccgaggaagaccgaaggaagaccacacgtgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgaacggagtgcgcgtgggcatcgcccgagttggagctggtacgggaactcatcgaactcaactggcatacccgcaatggtgaggtggaaccgcggcggatcgcgtacgaccgtgcccaggaggcct

SEQ ID NO: 8 - DNA, 20 bp, Artificial
  Forward primer for cloning of Bar gene
gctcgctgtcattttcgaga

SEQ ID NO: 9 - DNA, 20 bp, Artificial
  Reverse primer for cloning of Bar gene
aggcctcctgggcacggtcg

SEQ ID NO: 10 - DNA, 18 bp, Artificial
  Forward primer for amplification of DNA fragment use as “probe Bar”
agcgtggtcgctgtcatc

SEQ ID NO: 11 - DNA, 21 bp, Artificial
  Reverse primer for amplification of DNA fragment use as “probe Bar”
gttcgatgagttcccgtacca

SEQ ID NO: 12 - DNA, 20 bp, Artificial
  Forward primer for amplification of DNA fragment use as “probe AG S1-int2”
atcggcatcatcgtgtacct

SEQ ID NO: 13 - DNA, 18 bp, Artificial
  Reverse primer for amplification of DNA fragment use as “probe AG S1-int2”
cgccgatgctctcgctaa

SEQ ID NO: 14 - DNA, 247 bp, Artificial
  DNA sequence of Probe Bar
agcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgaacggagtgcgcgtgggcatcgcccgagttggagctggtacgggaactcatcgaac

SEQ ID NO: 15 - DNA, 628 bp, Artificial
  DNA sequence of Probe AGS1-int2
atcggcatcatcgtgtacctgggcctgcccgacttttaccggcagtcgcccgacaccatcccgggcttctacatctcgctggcgcggcgcaagatcgtgccgtgcttcttcgccatggtcatcatccaaaactactggatgtcggcgccgtacggccgcaactgggcctttttgttttcgtcgcagttcgtccccggctggggcgtcgtgctgctcgccatcttcttcctgctgggcctgtggtccgtcatcctctacgcgctcagccgcctggccgacgagcacacctggctgctccccatgctcgccatcggcctgtgcgccccacgctgggcccagatgctgtggggcgtctcgggcatgggcatgtacatcccctggatcccgaaccccgtcgctggcgccgtcatctcgcgctgcctgtggctgtggctcggcctgctcgacaacatccagggtgtcggcttcggcatgatgctgctcgccaccctgacccgcacccacgtcctcgccgtcctcatgggcggccaggtctttggttccgccttcaccatgctcgcccgcgccaccagccccaaccgcctcatccccaccgtcacctttcccgactttagcgagagcatcggcg

SEQ ID NO: 16 - DNA, 18 bp, Artificial
  Forward primer that amplify partial DNA sequence from MgAGS1
cctttgtcgcgccgtttg

SEQ ID NO: 17 - DNA, 20 bp, Artificial
  Reverse primer that amplify partial DNA sequence from MgAGS1
ccgtccttggtcgtagtgag

SEQ ID NO: 18 - DNA, 22 bp, Artificial
  Forward primer that amplify partial DNA sequence from MgFKS1
tggcatacaatttcgcagccgg

SEQ ID NO: 19 - DNA, 19 bp, Artificial
  Reverse primer that amplify partial DNA sequence from MgFKS1
ttgttatggcctttggtgg

SEQ ID NO: 20 - DNA, 24 bp, Artificial
  Forward primer that amplify partial DNA sequence from a gene encoding Actin
caactcgatcatgaagtgcgatgt

SEQ ID NO: 21 - DNA, 21 bp, Artificial
  Reverse primer that amplify partial DNA sequence from a gene encoding Actin
gctctcgtcgtactcctgctt

SEQ ID NO: 22 - DNA, 1251 bp, Artificial
  DNA sequence of EN4 promoter
gaattcgattatggatccggcgcgcctttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctaga

SEQ ID NO: 23 - DNA, 3882 bp, Bacillus circulans
atgcgcaccaagtatgttgcatggtcgctaatagcagccctgctaataaccacattgttccagtcagttggaccgggtgaaccggtagaggcggcaggagggccaaatctgacattaggcaaaaccataacggcaagcggccaatcccaaacctacagtccgaataatgtaaaggacagcaatcaaggcacttactgggagagcacgaataacgcttttccgcaatggattcaagcagatctgggcgcaagcaccagcatcgatcaaattgtattgaagcttcctgcgaactgggagactcggacgcagacgcttaccgttcaaggcagctcgaacggatcgacgtattcgacgatcgtcggatcggcgaattatgtattcaatcccgctgtagcgggaaactccgtgacgatcaactttgatccgacgagcacgcgctacgtccgacttcaagtgacgaacaatacaggctggccagctgcgcagctttccgaattcgagatctatggcgcaagcggcccgacgccgacgccttctgcgactccgcagccgtcaggcacgtatcaagcggaagcggctgccctctccggcggagccaaagtcaataccgatcatagcggctacgccggcacaggcttcgtggacggctattggactcaaggcgcggctacaacctttactgtacaggcgcaagcgggcggcaatcataatgtgacgctgaaatatgccaatgcgagcggaagcgacaagacgctcagcctctatgtgaacggtacaaagctccggcagacgaacttgcctaatctagcgaactgggatacttggggcaacaaagcggaaaccgtcaatttgaacgctgggtcgaataccatcgcgtacaaatatgattcaggcgattccggcaacgttaatcttgatcaaattacagtagaggcggctagctcaactccaacaccgactccgacatccacgccaacaccaacgccaacaccaacaccaacgccaacaccaacgccaacgccaacgccaacaccgacgccaacaccgactgcgacgccaacgccgcctccgggcagcaatattgcggtcggcaagccgatcaccgcatcctccagcaccttcacatttgtcgcggccaacgccaatgacaacagcaccgacacttattgggagggcgggggcaatccaagcacgctgacgcttgacttgggatcgaatcatgatgttacatccattgtgctgaagctgaatccttcaacggcatggagcacacgcactcaaacgattcaagtattggggcataatcaatcgacgacaaccttcagcaatttggtgtcgtctcagtcctataccttcaatccggcttcaggcaatacggtaacaattcctgttaccgcaacggtgaagcgcctgcagctgaatttcactgcaaattccggggcatcggcaggacaagtggcggaattccaagtattcggcacgccttctgctaatccggatctgatcattacagatatgtcttggtcgccggcatcgcctatcgagaccaatgccatcacgctgaatgccacggtcaaaaacaacggaacagctccttccggcgctacaacggtcaacttctatctgaacaatacgctggcgggctccgctcctgtcaacggtctggcagcgggcgcttcgactacggtatcgctgaacgcaggtacccgaactgcagcttcttataccgttaacgcgaaagtggatgagaacaattctgttatcgaattgaatgaatccaataacagctatacccatgcaacgccgctagttgttgcggctgtgtccagctccgacctcgtcgggaccgtatcctggacgccaagcaatccggtggcgggcaatgcagtcgcattgacggttaacttgaaaaaccaaggcaatatcgcatctgcgggcggcgctcacggtgtaacagtcgctctgaaaaatccggcaggctccacgctccaatcgttcagcggctcatacacgggcgtactcgctgcaggcgcctcggtcaacgtaacgcttccgggcacttggacggcttcgccaggcacttatacactgacgacgacgatcgcagcggatgggaacgaattgccgatcaagcagtccaacaatgtgagcaatacgaatctggttgtctatgcccagcgcggagccagcatgccgtatacccgctacgatacggatgacgctgcaagaggcggcggagctaccctgcaatctgcgcctaacttcgatcaagcgctgactgcttcggaagcatccggacagcgttatatcgcgcttccttcgaatggctcctacgcgcaatggaccattcgtccgggcgagggcggggacggtgtgacgatgagattcacgatgccggattccgctaatggcatgggccttaacggctcgcttgatgtctatgtcaacggagtaaaagccaagaccgtgccgcttacatcctattacagctggcagtacttctccagcgaccatccggctgatacgccggctggcggacgtccgctcttccgcttcgacgaagtgcactggaagatggatacgccgctgcagcctggagacacgatccgcattcagaagtcgggtgccgacagcctggaatatggagtagacttcctagagattgaagctgtgcctgccgccattgcgcgtccggcgaactcggtctccgtaactgatttcggggcagtagcgaatgacggccaagacgaccttgcagcgttcgaggcagctgttaatgcagctgtcacctccggcaagatcttgtatatcccggccggcacattccacctcggcaacatgtggaaaattggcagcgtagcgaacaaaatcaacaacattaccattatgggtgccggcatctggcataccaacattcagtttacgaatccgaatcaagcttccggcggcatttcgttccgcgtgacgggccagctggatttcagccatatctacatgaattccaacctgcgctcccgctatggcgagcaggctgtgtacaaagggtttatggataatttcgggaccaactccaaggtccacaatgtatgggtggagcatttcgaatgcggcttctgggtaggcgactatgctcatacgccagccattattgcgaacgggcttgtcattgagaacagccggatccgcaacaaccttgcagacggtgtcaacttcgcgcaaggcacgagcaactccacagtacgcaacagcagcatccgcaataacggcgacgacggcttggctgtctggaccagcaacgtgaatggcgctcctgcaggagtgaacaacacgttctcctacaatacgattgagaacaactggcgtgcagccggcatcgccttcttcggcggcagcggccacaaagcgacgcataatctcatcgtggatacggtcggcggctccgcaatccggatgaacacggtattcccaggctaccacttccagaacaacaccggcatcgtattctcggataccacgatcattaacagcggcaccagccgggatctgtacaacggcgagcgcggcgcgatagatctggaagcatccaatgatccgatcaaaaacgtcacgttcaccaatatcgatatcatcaacacgcagcggagcgccatccagttcggctacggcggcggcttcgagaatatcgtcttcaacaacatcaacattaacggcgccggcaaagacggcgttctgacttcacgcttctcgtcaccgcatcctggcgctgcgatctacacgtatacaggcaacggttctgccaccttcaacaatttgacaaccaatgatattgcacatccgaacctgtactttattcagaatggctttaatcttacaatccagtaa

SEQ ID NO: 24 - DNA, 122 bp, Nicotiana tabacum
tccattcaagatacaacatttctcctatagtcatgggatttgttctcttttcacaattgccttcatttcttcttgtctctacacttctcttattcctagtaatatcccactcttgccgtgcc

SEQ ID NO: 25 - DNA, 18 bp, Artificial
  primer
aaaacgtcacgttcacca

SEQ ID NO: 26 - DNA, 18 bp, Artificial
  primer
gttgaaggtggcagaacc

SEQ ID NO: 27 - DNA, 20 bp, Artificial
  primer
gtggtggccagtaagtcctc

SEQ ID NO: 28 - DNA, 21 bp, Artificial
  primer
ggacacaatgattagggatca

SEQ ID NO: 29 - DNA, 18 bp, Artificial
  primer
gcaagcgttgtccggatt

SEQ ID NO: 30 - DNA, 18 bp, Artificial
  primer
tacggcgtggactaccag

SEQ ID NO: 31 - protein, 1293 aa, Bacillus circulans
MRTKYVAWSLIAALLITTLFQSVGPGEPVEAAGGPNLTLGKTITASGQSQTYSPNNVKDSNQGTYWESTNNAFPQWIQADLGASTSIDQIVLKLPANWETRTQTLTVQGSSNGSTYSTIVGSANYVFNPAVAGNSVTINFDPTSTRYVRLQVTNNTGWPAAQLSEFEIYGASGPTPTPSATPQPSGTYQAEAAALSGGAKVNTDHSGYAGTGFVDGYWTQGAATTFTVQAQAGGNHNVTLKYANASGSDKTLSLYVNGTKLRQTNLPNLANWDTWGNKAETVNLNAGSNTIAYKYDSGDSGNVNLDQITVEAASSTPTPTPTSTPTPTPTPTPTPTPTPTPTPTPTPTPTATPTPPPGSNIAVGKPITASSSTFTFVAANANDNSTDTYWEGGGNPSTLTLDLGSNHDVTSIVLKLNPSTAWSTRTQTIQVLGHNQSTTTFSNLVSSQSYTFNPASGNTVTIPVTATVKRLQLNFTANSGASAGQVAEFQVFGTPSANPDLIITDMSWSPASPIETNAITLNATVKNNGTAPSGATTVNFYLNNTLAGSAPVNGLAAGASTTVSLNAGTRTAASYTVNAKVDENNSVIELNESNNSYTHATPLVVAAVSSSDLVGTVSWTPSNPVAGNAVALTVNLKNQGNIASAGGAHGVTVALKNPAGSTLQSFSGSYTGVLAAGASVNVTLPGTWTASPGTYTLTTTIAADGNELPIKQSNNVSNTNLVVYAQRGASMPYTRYDTDDAARGGGATLQSAPNFDQALTASEASGQRYIALPSNGSYAQWTIRPGEGGDGVTMRFTMPDSANGMGLNGSLDVYVNGVKAKTVPLTSYYSWQYFSSDHPADTPAGGRPLFRFDEVHWKMDTPLQPGDTIRIQKSGADSLEYGVDFLEIEAVPAAIARPANSVSVTDFGAVANDGQDDLAAFEAAVNAAVTSGKILYIPAGTFHLGNMWKIGSVANKINNITIMGAGIWHTNIQFTNPNQASGGISFRVTGQLDFSHIYMNSNLRSRYGEQAVYKGFMDNFGTNSKVHNVWVEHFECGFWVGDYAHTPAIIANGLVIENSRIRNNLADGVNFAQGTSNSTVRNSSIRNNGDDGLAVWTSNVNGAPAGVNNTFSYNTIENNWRAAGIAFFGGSGHKATHNLIVDTVGGSAIRMNTVFPGYHFQNNTGIVFSDTTIINSGTSRDLYNGERGAIDLEASNDPIKNVTFTNIDIINTQRSAIQFGYGGGFENIVFNNININGAGKDGVLTSRFSSPHPGAAIYTYTGNGSATFNNLTTNDIAHPNLYFIQNGFNLTIQ

SEQ ID NO: 32 - protein, 1260 aa, Bacillus circulans
MPNLTLGKTITASGQSQTYSPNNVKDSNQGTYWESTNNAFPQWIQADLGASTSIDQIVLKLPANWETRTQTLTVQGSSNGSTYSTIVGSANYVFNPAVAGNSVTINFDPTSTRYVRLQVTNNTGWPAAQLSEFEIYGASGPTPTPSATPQPSGTYQAEAAALSGGAKVNTDHSGYAGTGFVDGYWTQGAATTFTVQAQAGGNHNVTLKYANASGSDKTLSLYVNGTKLRQTNLPNLANWDTWGNKAETVNLNAGSNTIAYKYDSGDSGNVNLDQITVEAASSTPTPTPTSTPTPTPTPTPTPTPTPTPTPTPTPTPTATPTPPPGSNIAVGKPITASSSTFTFVAANANDNSTDTYWEGGGNPSTLTLDLGSNHDVTSIVLKLNPSTAWSTRTQTIQVLGHNQSTTTFSNLVSSQSYTFNPASGNTVTIPVTATVKRLQLNFTANSGASAGQVAEFQVFGTPSANPDLIITDMSWSPASPIETNAITLNATVKNNGTAPSGATTVNFYLNNTLAGSAPVNGLAAGASTTVSLNAGTRTAASYTVNAKVDENNSVIELNESNNSYTHATPLVVAAVSSSDLVGTVSWTPSNPVAGNAVALTVNLKNQGNIASAGGAHGVTVALKNPAGSTLQSFSGSYTGVLAAGASVNVTLPGTWTASPGTYTLTTTIAADGNELPIKQSNNVSNTNLVVYAQRGASMPYTRYDTDDAARGGGATLQSAPNFDQALTASEASGQRYIALPSNGSYAQWTIRPGEGGDGVTMRFTMPDSANGMGLNGSLDVYVNGVKAKTVPLTSYYSWQYFSSDHPADTPAGGRPLFRFDEVHWKMDTPLQPGDTIRIQKSGADSLEYGVDFLEIEAVPAAIARPANSVSVTDFGAVANDGQDDLAAFEAAVNAAVTSGKILYIPAGTFHLGNMWKIGSVANKINNITIMGAGIWHTNIQFTNPNQASGGISFRVTGQLDFSHIYMNSNLRSRYGEQAVYKGFMDNFGTNSKVHNVWVEHFECGFWVGDYAHTPAIIANGLVIENSRIRNNLADGVNFAQGTSNSTVRNSSIRNNGDDGLAVWTSNVNGAPAGVNNTFSYNTIENNWRAAGIAFFGGSGHKATHNLIVDTVGGSAIRMNTVFPGYHFQNNTGIVFSDTTIINSGTSRDLYNGERGAIDLEASNDPIKNVTFTNIDIINTQRSAIQFGYGGGFENIVFNNININGAGKDGVLTSRFSSPHPGAAIYTYTGNGSATFNNLTTNDIAHPNLYFIQNGFNLTIQ

SEQ ID NO: 33 - DNA, 3783 bp, Bacillus circulans
atgccaaatctgacattaggcaaaaccataacggcaagcggccaatcccaaacctacagtccgaataatgtaaaggacagcaatcaaggcacttactgggagagcacgaataacgcttttccgcaatggattcaagcagatctgggcgcaagcaccagcatcgatcaaattgtattgaagcttcctgcgaactgggagactcggacgcagacgcttaccgttcaaggcagctcgaacggatcgacgtattcgacgatcgtcggatcggcgaattatgtattcaatcccgctgtagcgggaaactccgtgacgatcaactttgatccgacgagcacgcgctacgtccgacttcaagtgacgaacaatacaggctggccagctgcgcagctttccgaattcgagatctatggcgcaagcggcccgacgccgacgccttctgcgactccgcagccgtcaggcacgtatcaagcggaagcggctgccctctccggcggagccaaagtcaataccgatcatagcggctacgccggcacaggcttcgtggacggctattggactcaaggcgcggctacaacctttactgtacaggcgcaagcgggcggcaatcataatgtgacgctgaaatatgccaatgcgagcggaagcgacaagacgctcagcctctatgtgaacggtacaaagctccggcagacgaacttgcctaatctagcgaactgggatacttggggcaacaaagcggaaaccgtcaatttgaacgctgggtcgaataccatcgcgtacaaatatgattcaggcgattccggcaacgttaatcttgatcaaattacagtagaggcggctagctcaactccaacaccgactccgacatccacgccaacaccaacgccaacaccaacaccaacgccaacaccaacgccaacgccaacgccaacaccgacgccaacaccgactgcgacgccaacgccgcctccgggcagcaatattgcggtcggcaagccgatcaccgcatcctccagcaccttcacatttgtcgcggccaacgccaatgacaacagcaccgacacttattgggagggcgggggcaatccaagcacgctgacgcttgacttgggatcgaatcatgatgttacatccattgtgctgaagctgaatccttcaacggcatggagcacacgcactcaaacgattcaagtattggggcataatcaatcgacgacaaccttcagcaatttggtgtcgtctcagtcctataccttcaatccggcttcaggcaatacggtaacaattcctgttaccgcaacggtgaagcgcctgcagctgaatttcactgcaaattccggggcatcggcaggacaagtggcggaattccaagtattcggcacgccttctgctaatccggatctgatcattacagatatgtcttggtcgccggcatcgcctatcgagaccaatgccatcacgctgaatgccacggtcaaaaacaacggaacagctccttccggcgctacaacggtcaacttctatctgaacaatacgctggcgggctccgctcctgtcaacggtctggcagcgggcgcttcgactacggtatcgctgaacgcaggtacccgaactgcagcttcttataccgttaacgcgaaagtggatgagaacaattctgttatcgaattgaatgaatccaataacagctatacccatgcaacgccgctagttgttgcggctgtgtccagctccgacctcgtcgggaccgtatcctggacgccaagcaatccggtggcgggcaatgcagtcgcattgacggttaacttgaaaaaccaaggcaatatcgcatctgcgggcggcgctcacggtgtaacagtcgctctgaaaaatccggcaggctccacgctccaatcgttcagcggctcatacacgggcgtactcgctgcaggcgcctcggtcaacgtaacgcttccgggcacttggacggcttcgccaggcacttatacactgacgacgacgatcgcagcggatgggaacgaattgccgatcaagcagtccaacaatgtgagcaatacgaatctggttgtctatgcccagcgcggagccagcatgccgtatacccgctacgatacggatgacgctgcaagaggcggcggagctaccctgcaatctgcgcctaacttcgatcaagcgctgactgcttcggaagcatccggacagcgttatatcgcgcttccttcgaatggctcctacgcgcaatggaccattcgtccgggcgagggcggggacggtgtgacgatgagattcacgatgccggattccgctaatggcatgggccttaacggctcgcttgatgtctatgtcaacggagtaaaagccaagaccgtgccgcttacatcctattacagctggcagtacttctccagcgaccatccggctgatacgccggctggcggacgtccgctcttccgcttcgacgaagtgcactggaagatggatacgccgctgcagcctggagacacgatccgcattcagaagtcgggtgccgacagcctggaatatggagtagacttcctagagattgaagctgtgcctgccgccattgcgcgtccggcgaactcggtctccgtaactgatttcggggcagtagcgaatgacggccaagacgaccttgcagcgttcgaggcagctgttaatgcagctgtcacctccggcaagatcttgtatatcccggccggcacattccacctcggcaacatgtggaaaattggcagcgtagcgaacaaaatcaacaacattaccattatgggtgccggcatctggcataccaacattcagtttacgaatccgaatcaagcttccggcggcatttcgttccgcgtgacgggccagctggatttcagccatatctacatgaattccaacctgcgctcccgctatggcgagcaggctgtgtacaaagggtttatggataatttcgggaccaactccaaggtccacaatgtatgggtggagcatttcgaatgcggcttctgggtaggcgactatgctcatacgccagccattattgcgaacgggcttgtcattgagaacagccggatccgcaacaaccttgcagacggtgtcaacttcgcgcaaggcacgagcaactccacagtacgcaacagcagcatccgcaataacggcgacgacggcttggctgtctggaccagcaacgtgaatggcgctcctgcaggagtgaacaacacgttctcctacaatacgattgagaacaactggcgtgcagccggcatcgccttcttcggcggcagcggccacaaagcgacgcataatctcatcgtggatacggtcggcggctccgcaatccggatgaacacggtattcccaggctaccacttccagaacaacaccggcatcgtattctcggataccacgatcattaacagcggcaccagccgggatctgtacaacggcgagcgcggcgcgatagatctggaagcatccaatgatccgatcaaaaacgtcacgttcaccaatatcgatatcatcaacacgcagcggagcgccatccagttcggctacggcggcggcttcgagaatatcgtcttcaacaacatcaacattaacggcgccggcaaagacggcgttctgacttcacgcttctcgtcaccgcatcctggcgctgcgatctacacgtatacaggcaacggttctgccaccttcaacaatttgacaaccaatgatattgcacatccgaacctgtactttattcagaatggctttaatcttacaatccagtaa